(19) United States
(12) Patent Application Publication
(10) Pub. No.: US 2013/0296316 A1
(43) Pub. Date: Nov. 7, 2013

(54) Antiparasitic Agents Based On mTOR Inhibitors

(71) Applicants: Michael P. Pollastri, Waltham, MA (US); Miguel Navarro, Granada (ES); Stephen Beverley, St. Louis, MO (US); Ana Rodriguez, New York, NY (US)

(72) Inventors: Michael P. Pollastri, Waltham, MA (US); Miguel Navarro, Granada (ES); Stephen Beverley, St. Louis, MO (US); Ana Rodriguez, New York, NY (US)

(21) Appl. No.: 13/808,892

(22) PCT Filed: Jul. 11, 2011
(86) PCT No.: PCT/US2011/043557

§ 371 Date: Jun. 17, 2013

Related U.S. Application Data
(60) Provisional application No. 61/362,930, filed on Jul. 9, 2010.

(51) Int. Cl. A61K 31/5377; A61K 31/519; A61K 31/496; A61K 31/4745

(52) U.S. Cl. CPC A61K 31/5377; A61K 31/4745; A61K 31/519; A61K 31/496
USPC 514/232.5; 514/293; 514/262.1; 514/233.5; 514/254.11; 514/232.8; 514/234.2; 514/233.2; 514/267

(57) ABSTRACT

Disclosed is the use of mammalian target of rapamycin (mTOR) and/or phosphoinositide-3-kinase (PI3K) inhibitors as antiparasitic drugs, particularly in those parasitic infections caused by trypanosomatid parasites {Trypanosoma sp. and Leishmania sp.). These inhibitors are useful as trypanocides.

### CROSS REFERENCE TO RELATED APPLICATIONS

This application claims the benefit of U.S. Provisional Application No. 61/362,930, filed Jul. 9, 2010, the entire contents of which are hereby incorporated by reference herein. Because Jul. 9, 2011 fell on a Saturday, this application properly claims the benefit of the provisional application.

### GRANTS

The following grants supported the disclosure herein: National Institutes of Health, 1F32AI091219-01 and National Institutes of Health, RC4 application. Thus, the U.S. government has certain rights to this disclosure.

### BACKGROUND

Human African trypanosomiasis (HAT) or African sleeping sickness, visceral and cutaneous leishmaniase (VL and CL, respectively), and Chagas disease (resulting from Trypanosoma brucei, T. cruzi, and Leishmania sp.) are a constellation of parasitic diseases that represent a significant health problem in the developing world. The World Health Organization estimates that over 22 million people are currently infected with one of these illnesses annually, all of which are debilitating and potentially fatal unless treated. However, current therapeutic interventions are significantly lacking, all with limited efficacy or life-threatening side effects. There are clear Target Product Profiles (TPPs) for new treatments, but a disproportionately small fraction of biomedical research is directed at the identification of such compounds. Since these diseases predominantly affect the very poor, they are recognized by WHO and the US Food and Drug Administration as “neglected diseases” in need of improved interventions.

Transmitted by the bite of infected insects, these diseases are treated by agents that are far from optimal in terms of safety, efficacy, and dosing methods. Currently, Stage I HAT is treated with two different drugs. The first is suramin, which has been used since the 1920s, administered by IV infusion over 30 days. The molecular target and mechanism of action for suramin are unknown and patients typically display detrimental side effects such as kidney failure, anaphylaxis, or neurological effects. The other Stage I treatment is pentamidine, which was first introduced in the 1940s and, similar to suramine, its mechanism of action is unknown. Pentamidine has fewer side effects, but requires intramuscular injection for 7 days. Neither of these drugs are effective against Stage II as they are incapable of crossing the blood-brain barrier (BBB).

There are two currently approved treatments for Stage II HAT. The first is melarsoprol, an organo-arsenic compound which is capable of crossing the BBB. Its mechanism of action is unknown and it is quite effective in treating HAT, but there are severe side effects such as cardiac arrhythmias and post-treatment reactive encephalopathy (PTRE). These side effects contribute to a 5% fatality rate in treated patients. The other treatment option for Stage II HAT is eflornithine, an irreversible ornothine decarboxylase inhibitor, which requires daily IV infusions up to 400 mg/kg per day for three weeks. Eflornithine has serious side effects, including anemia and convulsions, and is expensive to produce, leading to a large cost for treatment.

Similarly, VL has four treatment options: pentavalent antimonials, various formulations of amphotericin B, miltefosine, and paromomycin, all of which have problems with efficacy and toxicity. Only miltefosine can be administered orally. Antimonials are very toxic with side effects ranging from cardiac arrhythmia to pancreatitis. Amphotericin B treatment is very effective, but requires 30 days of hospitalization for clinical observation, a requirement that is not practical or even possible in some of the poorest nations. Miltefosine is advantageous due to its oral bioavailability, but is not safe for women who are or may become pregnant. Paromomycin is safe and inexpensive, but requires injections for 21 days.

Chagas disease therapeutics are principally the nitroaromatic compounds benznidazole and nifurtimox. Although both are orally-administered drugs, they require lengthy treatments and have significant side effects.

Thus, new treatments are needed for all three types of parasitic disease.

### SUMMARY

Disclosed is the use of mammalian target of rapamycin (mTOR) and/or phosphoinositide-3-kinase (PI3K) inhibitors as antiparasitic drugs, particularly in those parasitic infections caused by trypanosomatid parasites (Trypanosoma sp. and Leishmania sp.). These inhibitors are useful as trypanocides.

In one aspect, a method of treating a disease caused by a trypanosomatid parasite is described, comprising administering a therapeutically effective amount of an mTOR and/or PI3K inhibitor compound to a subject in need of treatment, wherein the compound has the structure of Formula (I),

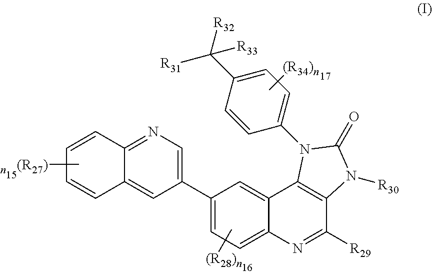

or a pharmaceutically acceptable salt or hydrate thereof,

wherein

R₂₇, R₂₈, R₂₉, and R₃₄ are each independently hydrogen, halogen, OH, CF₃, C₁-C₄ alkyl, ORₐ, OC(O)Rₐ, NRₐR_{b}, NRₐC(O)Rₐ, NRₐC(O)ORₐ, C(O)Rₐ, or C(O)NRₐR_{b};

R₃₀ is hydrogen, C₁-C₄ alkyl, aryl, heteroaryl, alkylaryl, alkylheteroaryl, C(O)Rₐ, C(O)ORₐR_{b}, or C(O)NRₐR_{b};

R₃₁ is hydrogen, halogen, OH, CN, CF₃, C₁-C₄ alkyl, ORₐ, OC(O)Rₐ, NRₐR_{b}, NRₐC(O)Rₐ, NRₐC(O)ORₐ, C(O)Rₐ, or C(O)NRₐR_{b};

R₃₂ and R₃₃ are each independently hydrogen or C₁-C₄ alkyl;

Rₐ and R_{b} are each independently hydrogen, C₁-C₄ alkyl, benzyl, pyridin-3-ylmethyl, —O—C₁-C₄ alkyl, or —C₁-C₄ alkyl-O—C₁-C₄ alkyl; and

n₁₅, n₁₆, and n₁₇ are each independently 0, 1, 2, 3, or 4.

In another aspect, a method of treating a disease caused by a trypanosomatid parasite is described, comprising administering a therapeutically effective amount of an mTOR and/or PI3K inhibitor compound to a subject in need of treatment, wherein the compound has the structure of Formula (VI),

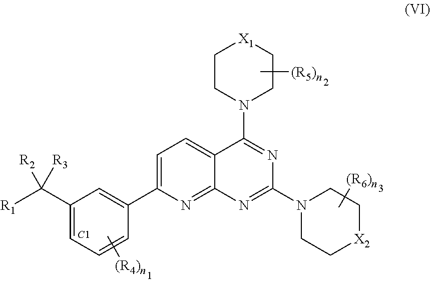

or a pharmaceutically acceptable salt or hydrate thereof,
wherein

R₁, R₂, R₃, and R₄ are each independently hydrogen, halogen, OH, CF₃, C₁-C₄ alkyl, ORₐ, NRₐR_{b}, C(O)Rₐ, or C(O)NRₐR_{b};

R₅ and R₆ are each independently hydrogen or C₁-C₄ alkyl;

X₁ and X₂ are each independently CRₐR_{b}, O, S, NRₐ, NC(O)Rₐ, or NC(O)ORₐ;

Rₐ and R_{b} are each independently hydrogen, C₁-C₄ alkyl, benzyl, pyridin-3-ylmethyl, —O—C₁-C₄ alkyl, or —C₁-C₄ alkyl O—C₁-C₄ alkyl; and

n₁, n₂, and n₃ are each independently 0, 1, 2, 3, or 4.

In yet another aspect, a method of treating a disease caused by a trypanosomatid parasite is described, comprising administering a therapeutically effective amount of an mTOR and/or PI3K inhibitor compound to a subject in need of treatment, wherein the compound has the structure of Formula (II),

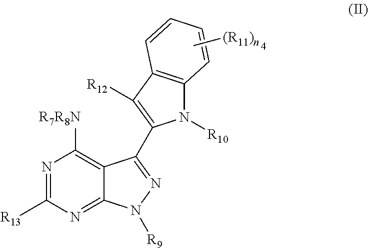

or a pharmaceutically acceptable salt or hydrate thereof,

wherein

R₇, R₈, R₉, and R₁₀ are each independently hydrogen, C₁-C₄ alkyl, aryl, heteroaryl, alkylaryl, alkylheteroaryl, C(O)Rₐ, C(O)ORₐ, or C(O)NRₐR_{b};

R₁₁, R₁₂ and R₁₃ are each independently hydrogen, halogen, OH, CF₃, C₁-C₄ alkyl, ORₐ, NRₐR_{b}, C(O)Rₐ, or C(O)NRₐR_{b};

Rₐ and R_{b} are each independently hydrogen, C₁-C₄ alkyl, benzyl, pyridin-3-ylmethyl, —O—C₁-C₄ alkyl; and

n₄ is 0, 1, 2, 3, or 4.

In yet another aspect, a method of treating a disease caused by a trypanosomatid parasite is described, comprising administering a therapeutically effective amount of an mTOR and/or PI3K inhibitor compound to a subject in need of treatment, wherein the compound has the structure of Formula (III),

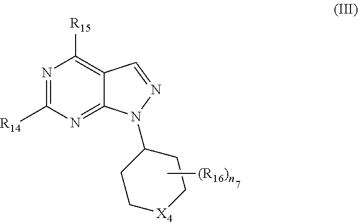

or a pharmaceutically acceptable salt or hydrate thereof,
wherein

R₁₄ is hydrogen, —C₁-C₄ alkyl-NRₐC(O)ORₐ, or

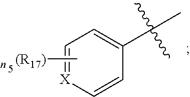

R₁₅ is hydrogen, C₁-C₄ alkyl, aryl, heteroaryl, NRₐR_{b}, or

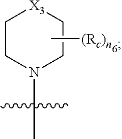

R_{c} and R₁₆ are each independently hydrogen or C₁-C₄ alkyl;

R₁₇ is hydrogen, halogen, OH, CF₃, C₁-C₄ alkyl, ORₐ, OC(O)Rₐ, NRₐR_{b}, NRₐC(O)Rₐ, NRₐC(O)ORₐ, C(O)Rₐ, or C(O)NRₐR_{b};

Rₐ and R_{b} are each independently hydrogen, C₁-C₄ alkyl, benzyl, pyridin-3-ylmethyl, —O—C₁-C₄ alkyl, or —C₁-C₄ alkyl O—C₁-C₄ alkyl;

X is C or N;

X₃ and X₄ are each independently CRₐR_{b}, O, S, NRₐ, NC(O)Rₐ, or NC(O)ORₐ; and

n₅, n₆, and n₇ are each independently 0, 1, 2, 3, or 4.

In yet another aspect, a method of treating a disease caused by a trypanosomatid parasite is described, comprising administering a therapeutically effective amount of an mTOR and/or PI3K inhibitor compound to a subject in need of treatment, wherein the compound has the structure of Formula (IV),

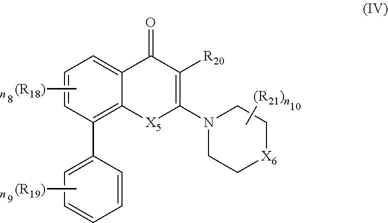

or a pharmaceutically acceptable salt or hydrate thereof,
wherein

R₁₈, R₁₉, and R₂₀ are each independently hydrogen, halogen, OH, CF₃, C₁-C₄ alkyl, ORₐ, OC(O)Rₐ, NRₐR_{b}, NRₐC(O)Rₐ, NRₐC(O)ORₐ, C(O)Rₐ, or C(O)NRₐR_{b};

R₂₁ is hydrogen or C₁-C₄ alkyl;

X₅ and X₆ are each independently CRₐR_{b}, O, S, NRₐ, NC(O)Rₐ, or NC(O)ORₐ;

Rₐ and R_{b} are each independently hydrogen, C₁-C₄ alkyl, benzyl, pyridin-3-ylmethyl, —O—C₁-C₄ alkyl, or —C₁-C₄ alkyl O—C₁-C₄ alkyl; and

n₈, n₉, and n₁₀ are each independently 0, 1, 2, 3, or 4.

In yet another aspect, a method of treating a disease caused by a trypanosomatid parasite is described, comprising administering a therapeutically effective amount of an mTOR and/or PI3K inhibitor compound to a subject in need of treatment, wherein the compound has the structure of Formula (V),

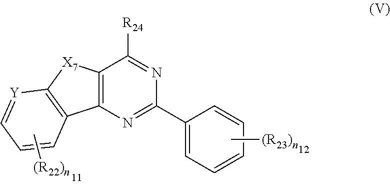

or a pharmaceutically acceptable salt or hydrate thereof,
wherein

R₂₄ is hydrogen, C₁-C₄ alkyl, aryl, heteroaryl, NRₐR_{b},

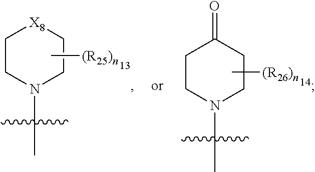

R₂₂ and R₂₃ are each independently hydrogen, halogen, OH, CF₃, C₁-C₄ alkyl, ORₐ, C₁-C₄ alkyl-ORₐ, OC(O)Rₐ, NRₐR_{b}, NRₐC(O)Rₐ, NRₐC(O)ORₐ, C(O)Rₐ, or C(O)NRₐR_{b};

R₂₅ and R₂₆ are each independently hydrogen or C₁-C₄ alkyl;

X₇ and X₈ are each independently CRₐR_{b}, O, S, NRₐ, NC(O)Rₐ, or NC(O)ORₐ;

Rₐ and R_{b} are each independently hydrogen, C₁-C₄ alkyl, benzyl, pyridin-3-ylmethyl, —O—C₁-C₄ alkyl, or —C₁-C₄ alkyl-O—C₁-C₄ alkyl;

Y is C or N; and

n₁₁, n₁₂, n₁₃, and n₁₄ are each independently 0, 1, 2, 3, or 4.

In yet another aspect, a method of treating a disease caused by a trypanosomatid parasite is described, comprising administering a therapeutically effective amount of an mTOR and/or PI3K inhibitor compound to a subject in need of treatment, wherein the compound has the structure of Formula (VII),

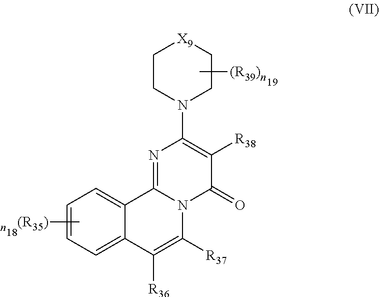

or a pharmaceutically acceptable salt or hydrate thereof,
wherein

R₃₅, R₃₆, R₃₇, and R₃₈ are each independently hydrogen, halogen, OH, CF₃, C₁-C₄ alkyl, ORₐ, OC(O)Rₐ, NRₐR_{b}, NRₐC(O)Rₐ, NRₐC(O)ORₐ, C(O)Rₐ, or C(O)NRₐR_{b};

R₃₉ is hydrogen or C₁-C₄ alkyl;

X₉ is CRₐR_{b}, O, S, NRₐ, NC(O)Rₐ, or NC(O)ORₐ;

Rₐ and R_{b} are each independently hydrogen, C₁-C₄ alkyl, benzyl, pyridin-3-ylmethyl, —O—C₁-C₄ alkyl, or —C₁-C₄ alkyl O—C₁-C₄ alkyl; and

n₁₈ and n₁₉ are each independently 0, 1, 2, 3, or 4.

### BRIEF DESCRIPTION OF THE FIGURES

The following figures are presented for the purpose of illustration only, and are not intended to be limiting.

FIGS. 1A and 1B are graphic representations of EC₅₀ values for the indicated compounds in T. brucei rhodesiense. FIG. 1A shows EC₅₀ values for compounds WYE-354, PP242, and PI-103. FIG. 1B shows EC₅₀ values for NVP-BEZ-235.

FIG. 2A shows compounds for treatment of Stage I and Stage II trypanosomiasis.

FIG. 2B shows compounds for treatment of leishmaniasis.

FIG. 3 shows the chemical structure of rapamycin.

FIG. 4 depicts representative kinase domain mTOR inhibitors selected for screening.

FIG. 5 depicts various mTOR inhibitor analogs designed.

FIG. 6 depicts a scheme for the synthesis of an mTOR inhibitor precursor.

FIG. 7 depicts synthesis schemes for mTOR inhibitor Analogs 1-6 and 8.

FIG. 8 depicts additional mTOR inhibitor analogs.

FIG. 9 depicts synthesis schemes for mTOR inhibitor Analogs 14, 17, 19 and 21.

FIG. 10 depicts dose response curves of the several active inhibitors. PI-103, WYE-354, Pp242 and NVP-BEZ235 against (A) T. brucei rhodesiense, (B) Leishmania donovani axenic amastigotes, (C) Leishmania donovani promastigotes, (D) Leishmania major promastigotes, (E), NVP-BEZ235 against T. cruzi, and (F) T. brucei rhodesiense and gambiense.

FIG. 11 shows phenotypic observations of parasites upon dosage with NVP-BEZ235.

FIG. 12 shows trypanocidal activity of NVP-BEZ235 in an acute mouse infection model.

### DESCRIPTION

All publications, patent applications, patents, and other references mentioned herein are incorporated by reference in their entirety. In case of conflict, the present specification, including definitions, will control. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

The trypanosomatids Trypanosoma brucei and Leishmania major possess multiple targets of rapamycin (TOR) enzymes that are essential for cell growth and virulence. The mammalian homolog, mTOR, has been the focus of treatments for cancer. It has been discovered that these mTOR inhibitor chemotypes are useful for the treatment of trypanosomiasis and leishmaniasis and killing the trypanosomatids responsible, at least in part, for these diseases.

Target repurposing utilizes knowledge of ‘druggable’ targets obtained in one organism and exploits this information to pursue new drug targets in other organisms. This disclosure shows the effect of inhibitors targeting the kinase domain of the mammalian Target of Rapamycin (mTOR) and human phosphoinositide-3-kinases (PI3Ks) against the kinetoplastid parasites Trypanosoma brucei, T. cruzi, Leishmania major, and L. donovani. The genomes of trypanosomatids encode at least 12 proteins belonging to the PI3K protein superfamily, some of which are unique to parasites. Moreover, the shared PI3Ks differ greatly in sequence from those of the human host, thereby providing opportunities for selective inhibition. Several inhibitors showed micromolar or better efficacy against these organisms in culture. One compound, NVP-BEZ235, displayed high potency (sub-nanomolar) efficacy against cultured parasites, and the ability to clear parasitemia in an animal model of T. brucei rhodesiense infection.

The disclosure shows that mammalian PI3/TOR kinase inhibitors are useful for anti-trypanosomal drug discovery and treatment. In one embodiment, NVP-BEZ235, an advanced clinical candidate against solid tumors, is useful as an agent for treating African sleeping sickness.

### DEFINITIONS

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The terms “alkyl” and “alk” refer to a straight or branched chain alkane (hydrocarbon) radical containing from 1 to 12 carbon atoms, preferably 1 to 6 carbon atoms. Exemplary “alkyl” groups include methyl, ethyl, propyl, isopropyl, n-butyl, t-butyl, isobutyl pentyl, hexyl, isohexyl, heptyl, 4,4-dimethylpentyl, octyl, 2,2,4-trimethylpentyl, nonyl, decyl, undecyl, dodecyl, and the like. The term “(C₁-C₄)alkyl” refers to a straight or branched chain alkane (hydrocarbon) radical containing from 1 to 4 carbon atoms, such as methyl, ethyl, propyl, isopropyl, n-butyl, t-butyl, and isobutyl. The terms “alkyl” and “alk” may be optionally substituted. “Substituted alkyl” refers to an alkyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. In the aforementioned exemplary substitutents, groups such as alkyl, cycloalkyl, alkenyl, alkynyl, cycloalkenyl, heterocycle and aryl can themselves be optionally substituted. The terms “alkylaryl” refers to alkyl group substituted by one or more aryl group. The terms “alkylheteroaryl” refers to alkyl group substituted by one or more heteroaryl group.

The term “alkenyl” refers to a straight or branched chain hydrocarbon radical containing from 2 to 12 carbon atoms and at least one carbon-carbon double bond. Exemplary such groups include ethenyl or allyl. The term “C₂-C₆ alkenyl” refers to a straight or branched chain hydrocarbon radical containing from 2 to 6 carbon atoms and at least one carbon-carbon double bond, such as ethylenyl, propenyl, 2-propenyl, (E)-but-2-enyl, (Z)-but-2-enyl, 2-methy(E)-but-2-enyl, 2-methy(Z)-but-2-enyl, 2,3-dimethyl-but-2-enyl, (Z)-pent-2-enyl, (E)-pent-1-enyl, (Z)-hex-1-enyl, (E)-pent-2-enyl, (Z)-hex-2-enyl, (E)-hex-2-enyl, (Z)-hex-1-enyl, (E)-hex-1-enyl, (Z)-hex-3-enyl, (E)-hex-3-enyl, and (E)-hex-1,3-dienyl. The term “alkenyl” may be optionally substituted. “Substituted alkenyl” refers to an alkenyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substitutents can themselves be optionally substituted.

The term “alkynyl” refers to a straight or branched chain hydrocarbon radical containing from 2 to 12 carbon atoms and at least one carbon to carbon triple bond. Exemplary such groups include ethynyl. The term “C₂-C₆ alkynyl” refers to a straight or branched chain hydrocarbon radical containing from 2 to 6 carbon atoms and at least one carbon-carbon triple bond, such as ethynyl, prop-1-ynyl, prop-2-ynyl, but-1-ynyl, but-2-ynyl, pent-1-ynyl, pent-2-ynyl, hex-1-ynyl, hex-2-ynyl, hex-3-ynyl. The term “alkynyl” may be optionally substituted. “Substituted alkynyl” refers to an alkynyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}C(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substitutents can themselves be optionally substituted.

The term “cycloalkyl” refers to a fully saturated cyclic hydrocarbon group containing from 1 to 4 rings and 3 to 8 carbons per ring. “C₃-C₇ cycloalkyl” refers to cyclopropyl, cyclobutyl, cyclopentyl, cyclohexyl, or cycloheptyl. The term “cycloalkyl” may be optionally substituted. “Substituted cycloalkyl” refers to a cycloalkyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{J}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substitutents can themselves be optionally substituted. Exemplary substituents also include spiro-attached or fused cylic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substitutents can themselves be optionally substituted.

The term “cycloalkenyl” refers to a partially unsaturated cyclic hydrocarbon group containing 1 to 4 rings and 3 to 8 carbons per ring. Exemplary such groups include cyclobutenyl, cyclopentenyl, cyclohexenyl, etc. The term “cycloalkenyl” may be optionally substituted. “Substituted cycloalkenyl” refers to a cycloalkenyl group substituted with one more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substitutents can themselves be optionally substituted. Exemplary substituents also include spiro-attached or fused cylic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “aryl” refers to cyclic, aromatic hydrocarbon groups that have 1 to 5 aromatic rings, especially monocyclic or bicyclic groups such as phenyl, biphenyl or naphthyl. Where containing two or more aromatic rings (bicyclic, etc.), the aromatic rings of the aryl group may be joined at a single point (e.g., biphenyl), or fused (e.g., naphthyl, phenanthrenyl and the like). The term “aryl” may be optionally substituted. “Substituted aryl” refers to an aryl group substituted by one or more substituents, preferably 1 to 3 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{C}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substitutents can themselves be optionally substituted. Exemplary substituents also include fused cylic groups, especially fused cycloalkyl, fused cycloalkenyl, fused heterocycle, or fused aryl, where the aforementioned cycloalkyl, cycloalkenyl, heterocycle and aryl substituents can themselves be optionally substituted.

The terms “heterocycle” and “heterocyclic” refer to fully saturated, or partially or fully unsaturated, including aromatic (i.e., “heteroaryl”) cyclic groups (for example, 4 to 7 membered monocyclic, 7 to 11 membered bicyclic, or 8 to 16 membered tricyclic ring systems) which have at least one heteroatom in at least one carbon atom-containing ring. Each ring of the heterocyclic group containing a heteroatom may have 1, 2, 3, or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. (The term “heteroarylium” refers to a heteroaryl group bearing a quaternary nitrogen atom and thus a positive charge.) The heterocyclic group may be attached to the remainder of the molecule at any heteroatom or carbon atom of the ring or ring system. Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include indolyl, isoindolyl, benzothiazolyl, benzoxazolyl, benzoxadiazolyl, benzothienyl, benzo[d][1,3]dioxolyl, 2,3-dihydrobenzo[b][1,4]dioxinyl, quinuclidinyl, quinolinyl, tetrahydroisoquinolinyl, isoquinolinyl, benzimidazolyl, benzopyranyl, indolizinyl, benzofuryl, benzofurazanyl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl and the like. Exemplary tricyclic heterocyclic groups include carbazolyl, benzidolyl, phenanthrolinyl, acridinyl, phenanthridinyl, xanthenyl and the like. The terms “heterocycle” and “heterocyclic” may be optionally substituted.

“Substituted heterocycle” and “substituted heterocyclic” (such as “substituted heteroaryl”) refer to heterocycle or heterocyclic groups substituted with one or more substituents, preferably 1 to 4 substituents, at any available point of attachment. Exemplary substituents include but are not limited to one or more of the following groups: hydrogen, halogen (e.g., a single halogen substituent or multiple halo substitutents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substitutents can themselves be optionally substituted. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “alkylamino” refers to a group having the structure —NHR′, wherein R′ is hydrogen, alkyl or substituted alkyl, cycloalkyl or substituted cyclolakyl, as defined herein. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexylamino, and the like.

The term “dialkylamino” refers to a group having the structure —NRR′, wherein R and R′ are each independently alkyl or substituted alkyl, cycloalkyl or substituted cycloalkyl, cycloalkenyl or substituted cyclolalkenyl, aryl or substituted aryl, heterocylyl or susbstituted heterocyclyl, as defined herein. R and R′ may be the same or different in an dialkyamino moiety. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(tert-butyl)amino, di(neopentyl)amino, di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of cyclic diaminoalkyl groups include, but are not limited to, aziridinyl, pyrrolidinyl, piperidinyl, morpholinyl, pyrrolyl, imidazolyl, 1,3,4-trianolyl, and tetrazolyl.

The terms “halogen” or “halo” refer to chlorine, bromine, fluorine or iodine.

Unless otherwise indicated, any heteroatom with unsatisfied valences is assumed to have hydrogen atoms sufficient to satisfy the valences.

The compounds used in the disclosed methods may form salts which are also within the scope of this invention. Reference to a compound of the present invention is understood to include reference to salts thereof, unless otherwise indicated. The term “salt(s)”, as employed herein, denotes acidic and/or basic salts formed with inorganic and/or organic acids and bases. In addition, when a compound of the present invention contains both a basic moiety, such as but not limited to a pyridine or imidazole, and an acidic moiety such as but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation. Salts of the compounds used in the disclosed methods may be formed, for example, by reacting a compound described herein with an amount of acid or base, such as an equivalent amount, in a medium such as one in which the salt precipitates or in an aqueous medium followed by lyophilization.

The compounds used in the disclosed methods that contain a basic moiety, such as but not limited to an amine or a pyridine or imidazole ring, may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds used in the disclosed methods that contain an acidic moiety, such but not limited to a carboxylic acid, may form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glycamides, t-butyl amines, and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Prodrugs and solvates of the compounds of the invention are also contemplated herein. The term “prodrug” as employed herein denotes a compound that, upon administration to a subject, undergoes chemical conversion by metabolic or chemical processes to yield a compound of the present invention, or a salt and/or solvate thereof. Solvates of the compounds in the disclosure include, for example, hydrates.

Compounds of the present disclosure, and salts or solvates thereof, may exist in their tautomeric form (for example, as an amide or imino ether). All such tautomeric forms are contemplated herein as part of the present invention.

All stereoisomers of the present compounds (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the International Union of Pure and Applied Chemistry (IUPAC) 1974 Recommendations. The racemic forms can be resolved by physical methods, such as, for example, fractional crystallization, separation or crystallization of diastereomeric derivatives or separation by chiral column chromatography. The individual optical isomers can be obtained from the racemates by any suitable method, including without limitation, conventional methods, such as, for example, salt formation with an optically active acid followed by crystallization.

Compounds of the present disclosure are, subsequent to their preparation, preferably isolated and purified to obtain a composition containing an amount by weight equal to or greater than 90%, for example, equal to greater than 95%, equal to or greater than 99% of the compounds (“substantially pure” compounds), which is then used or formulated as described herein. Such “substantially pure” compounds are also contemplated herein as part of the present disclosure.

All configurational isomers of the compounds of the present disclosure are contemplated, either in admixture or in pure or substantially pure form. The definition of compounds of the present disclosure embraces both cis (Z) and trans (E) alkene isomers, as well as cis and trans isomers of cyclic hydrocarbon or heterocyclic rings.

Throughout the specifications, groups and substituents thereof may be chosen to provide stable moieties and compounds.

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference.

Certain compounds of the present disclosure may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

The present invention also includes isotopically labeled compounds, which are identical to the compounds disclosed herein, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, or an enantiomer, diastereomer, tautomer, or pharmaceutically acceptable salt or solvate thereof, which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds can generally be prepared by carrying out the procedures disclosed in the Schemes and/or in the Examples below, by substituting a readily available isotopically labeled reagent for a non-isotopically labeled reagent.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of infectious diseases or proliferative disorders. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

As used herein, “treat,” “treating” or “treatment” refers to administering a therapy in an amount, manner (e.g., schedule of administration), and/or mode (e.g., route of administration), effective to improve a disorder (e.g., a disorder described herein) or a symptom thereof, or to prevent or slow the progression of a disorder (e.g., a disorder described herein) or a symptom thereof. This can be evidenced by, e.g., an improvement in a parameter associated with a disorder or a symptom thereof, e.g., to a statistically significant degree or to a degree detectable to one skilled in the art. An effective amount, manner, or mode can vary depending on the subject and may be tailored to the subject. By preventing or slowing progression of a disorder or a symptom thereof, a treatment can prevent or slow deterioration resulting from a disorder or a symptom thereof in an affected or diagnosed subject.

The term “or” is used herein to mean, and is used interchangeably with, the term “and/or,” unless context clearly indicates otherwise.

The term “therapeutically effective amount” refers to an amount (e.g., dose) sufficient to achieve a desired therapeutic effect when treating a subject having a disorder or condition described herein. In certain embodiments, a “therapeutically effective amount” is an amount sufficient to achieve tissue or serum concentrations required to treat, halt progression of, or the worsening of diseases disclosed herein. It is also to be understood herein that a “therapeutically effective amount” may be interpreted as an amount giving a desired therapeutic effect, either taken in one dose or in any dosage or route, taken alone or in combination with other therapeutic agents.

The term “therapeutically effective amount of an mTOR and/or PI3K inhibitor compound” refers to at least one compound selected from the list disclosed herein.

As used herein, a “subject” is any subject for whom diagnosis, prognosis, or therapy is desired. In some embodiments, the subject is a mammal. For example, a subject can be a mammal such as a human, dog, cat, horse, cow, pig, or non-human primate, such as a monkey, chimpanzee, baboon or rhesus. In particular embodiments, the subject is a human. In other particular embodiments, the subject is a livestock or domestic animal.

### Target of Rapamycin (TOR)

Rapamycin is a natural cell growth inhibitor in mammals. TOR is a phosphoinositide-3-kinase (PI3K)-related kinase. T. brucei TOR (TbTOR1 and TbTOR2) are essential for cell growth and regulation. Tests show that Rapamycin inhibited T. brucei cell growth with an EC₅₀ of 152 nM by inhibiting TbTOR2 complex formation. L. major expressTOR-like proteins. Some kinase domain inhibitors are selective for mTOR; others are cross-reactive with related PI3Ks.

The disclosure provides methods of treating T. brucei brucei, T. brucei rhodesiense (human infective strain) and L. major promastigotes. In some embodiments, the methods further comprise administering one or more of the disclosed compounds in combination with pharmaceutically acceptable salt or carrier. In certain embodiments, such methods include administering one or more of the disclosed compounds to a subject. In some embodiments, the subject can be a mammal, e.g., a human, mouse, rat, guinea pig, dog, cat, horse, cow, pig, or non-human primate, such as a monkey, chimpanzee, baboon or rhesus. In particular embodiments, the subject is a human.

Although some of the compounds outlined in this disclosure are useful therapeutics for trypanosomal diseases, these compound classes can also be optimized to improve their parasite killing selectivity (over human cells), improve their pharmacokinetic profiles to be even more amenable to treatment of parasites (such as crossing the blood-brain barrier for T. brucei infections), entering macrophages (for Leishmania infections), etc. In some embodiments, the disclosure provides methods of killing a trypanosomatid parasite, comprising administering a therapeutically effective amount of an mTOR inhibitor compound to a subject infected with a trypanosomatid parasite.

### Trypanosomatid Genomes

Database mining of trypanosomatid genomes has revealed the presence of at least 12 proteins belonging to the PI3K protein superfamily (PFAM PF00454), many of which are unique to the parasites. Notably orthologous proteins are highly divergent from those of the human host. These include predicted kinases related to the eukaryotic class I and II PI3Ks, PI4Ks, and PIKKs including TOR, ATM and ATR ([26,27], and data not shown). Where tested, PI3Ks appear to be essential for viability and/or virulence in trypanosomatids. Two PIK subfamily members have been examined in T. brucei. The trypanosome Class III PI3K TbVps34 has an essential function in membrane trafficking and in Golgi segregation during cell division [28]. These authors suggested that, similar to yeast, T. brucei possesses only one genuine PI3K. TbPI4Kβ is also an essential protein in T. brucei, required for maintenance of Golgi structure, protein trafficking, and cytokinesis [28]. Trypanosomatids possess four distinct genes belonging to the TOR family, in contrast to mammals, which possess a single mTOR protein [29,30,31,32]. TORs act in concert with other proteins in complexes referred to as TORCs, which have different protein subunit compositions, and cellular functions [33].

In T. brucei, the two conserved signaling complexes, TORC1 and TORC2, whose functions appear analogous to that described in mammalian or yeast TORCs, mediate the essential functions of TOR1 and TOR2 for cell growth [32,34]. While TbTORC1 regulates protein synthesis, cell cycle progression and autophagy, TbTORC2 plays a key role in maintaining the polarization of the actin cytoskeleton, which is required for the proper functioning of endocytic processes, cell division, and cytokinesis [29,35]. Correspondingly, TOR1 and TOR2 are essential genes in Leishmania major [30]. Recent work has characterized a third TOR protein, TOR3, in Leishmania major and T. brucei, that is implicated in the formation of acidocalcisomes and participation in stress response [30,31]. A fourth TOR in T. brucei and Leishmania (TOR4) lacks the FRB domain responsible for binding rapamycin-binding proteins, yet possesses all other characteristic domains of TOR kinases [29,30].

The essentiality of several PIKs and TOR1 and TOR2 and the requirement for TOR3 for virulence in both trypanosomes and Leishmania provide genetic validation of these kinases as drug targets.

### Methods of Treatment

In one aspect, a method of treating a disease caused by a trypanosomatid parasite is described, comprising administering a therapeutically effective amount of an mTOR and/or PI3K inhibitor compound to a subject in need of treatment.

Methods are disclosed herein for treating a disease caused by a trypanosomatid parasite, comprising administering a therapeutically effective amount of an mTOR and/or PI3K inhibitor compound to a subject in need of treatment. In one aspect, such methods comprise administering the compound of Formulas (I), (II), (III), (IV), (V), (VI), and (VII) or a pharmaceutically acceptable salt or hydrate thereof to a subject.

As stated herein, a “therapeutically effective amount” refers to an amount (e.g., dose) effective in treating a subject, having a disorder or condition described herein. It is also to be understood herein that a “therapeutically effective amount” may be interpreted as an amount giving a desired therapeutic effect, either taken in one dose or in any dosage or route, taken alone or in combination with other therapeutic agents. In some embodiments, the dosage range is from about 0.01 to about 1000 mg/kg of body weight. In some embodiments, the dosage range is from about 0.5 to about 500 mg/kg of body weight. In some embodiments, the dosage range is from about 0.5 to about 50 mg/kg of body weight.

In one aspect, a method of treating a disease caused by a trypanosomatid parasite is described, comprising administering a therapeutically effective amount of an mTOR and/or P13K inhibitor compound to a subject in need of treatment, wherein the compound has the structure of Formula (I),

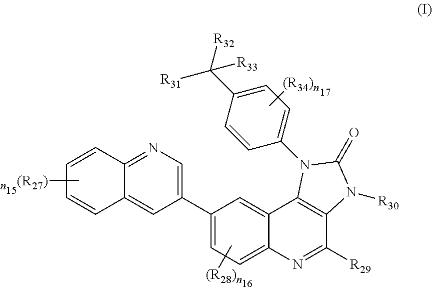

or a pharmaceutically acceptable salt or hydrate thereof,
wherein

R₂₇, R₂₈, R₂₉, and R₃₄ are each independently hydrogen, halogen, OH, CF₃, C₁-C₄ alkyl, ORₐ, OC(O)Rₐ, NRₐR_{b}, NRₐC(O)Rₐ, NRₐC(O)ORₐ, C(O)Rₐ, or C(O)NRₐR_{b};

R₃₀ is hydrogen, C₁-C₄ alkyl, aryl, heteroaryl, alkylaryl, alkylheteroaryl, C(O)Rₐ, C(O)ORₐR_{b}, or C(O)NRₐR_{b};

R₃₁ is hydrogen, halogen, OH, CN, CF₃, C₁-C₄ alkyl, ORₐ, OC(O)Rₐ, NRₐR_{b}, NRₐC(O)Rₐ, NRₐC(O)ORₐ, C(O)Rₐ, or C(O)NRₐR_{b};

R₃₂ and R₃₃ are each independently hydrogen or C₁-C₄ alkyl;

Rₐ and R_{b} are each independently hydrogen, C₁-C₄ alkyl, benzyl, pyridin-3-ylmethyl, —O—C₁-C₄ alkyl, or —C₁-C₄ alkyl-O—C₁-C₄ alkyl; and

n₁₅, n₁₆, and n₁₇ are each independently 0, 1, 2, 3, or 4.

In some embodiments, R₃₁ is CN. In some embodiments, R₃₂ and R₃₃ are each H. In some embodiments, R₃₀ is Me. In some embodiments, R₂₇ is OMe, OH, or OAc. In some embodiments, R₂₉ and R₃₄ are each H. In some embodiments, the compound is

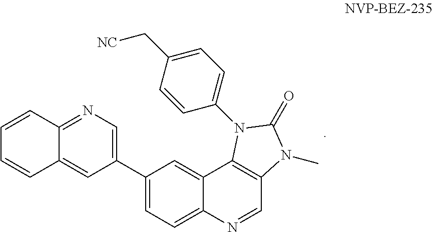 [SMILES: Cn1c(=O)n(-c2ccc(CC#N)cc2)c2c3cc(-c4cnc5ccccc5c4)ccc3ncc21]

In another aspect, a method of treating a disease caused by a trypanosomatid parasite is described, comprising administering a therapeutically effective amount of an mTOR and/or PI3K inhibitor compound to a subject in need of treatment, wherein the compound has the structure of Formula (VI),

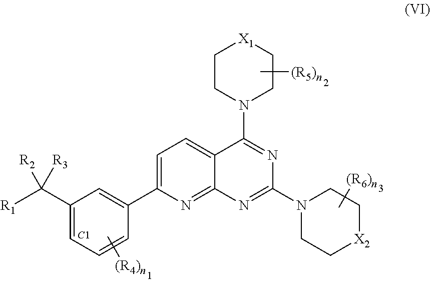

or a pharmaceutically acceptable salt or hydrate thereof,
wherein

R₁, R₂, R₃, and R₄ are each independently hydrogen, halogen, OH, CF₃, C₁-C₄ alkyl, ORₐ, NRₐR_{b}, C(O)Rₐ, or C(O)NRₐR_{b};

R₅ and R₆ are each independently hydrogen or C₁-C₄ alkyl;

X₁ and X₂ are each independently CRₐR_{b}, O, S, NRₐ, NC(O)Rₐ, or NC(O)ORₐ;

Rₐ and R_{b} are each independently hydrogen, C₁-C₄ alkyl, benzyl, pyridin-3-ylmethyl, —O—C₁-C₄ alkyl, or —C₁-C₄ alkyl O—C₁-C₄ alkyl; and

n₁, n₂, and n₃ are each independently 0, 1, 2, 3, or 4.

In some embodiments, X₁ and X₂ are each independently O. In some embodiments, R₄ is attached to the C1 position of Formula (VI). In some embodiments, R₄ is OMe. In some embodiments, R₆ is Me. In some embodiments, R₁ is OH. In some embodiments, the compound is

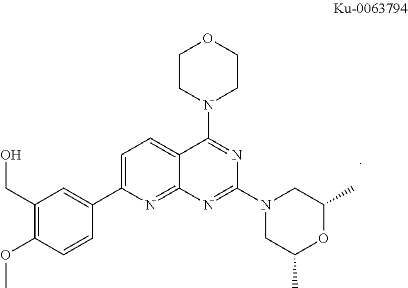 [SMILES: COc1ccc(-c2ccc3c(N4CCOCC4)nc(N4C[C@@H](C)O[C@@H](C)C4)nc3n2)cc1CO]

In yet another aspect, a method of treating a disease caused by a trypanosomatid parasite is described, comprising administering a therapeutically effective amount of an mTOR and/or PI3K inhibitor compound to a subject in need of treatment, wherein the compound has the structure of Formula (II),

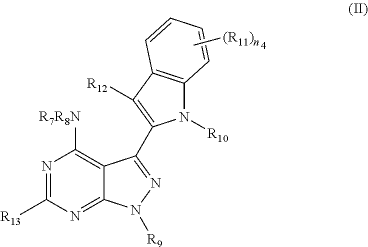

or a pharmaceutically acceptable salt or hydrate thereof,
wherein

R₇, R₈, R₉, and R₁₀ are each independently hydrogen, C₁-C₄ alkyl, aryl, heteroaryl, alkylaryl, alkylheteroaryl, C(O)Rₐ, C(O)ORₐ, or C(O)NRₐR_{b};

R₁₁, R₁₂ and R₁₃ are each independently hydrogen, halogen, OH, CF₃, C₁-C₄ alkyl, ORₐ, NRₐR_{b}, C(O)Rₐ, or C(O)NRₐR_{b};

Rₐ and R_{b} are each independently hydrogen, C₁-C₄ alkyl, benzyl, pyridin-3-ylmethyl, —O—C₁-C₄ alkyl; and

nₐ is 0, 1, 2, 3, or 4.

In some embodiments, R₇ and R₈ are H. In some embodiments, R₁₃ is H. In some embodiments, R₉ is methyl, ethyl, or isopropyl. In some embodiments, R₁₀ is H. In some embodiments, R₁₁ is OH. In some embodiments, the compound is

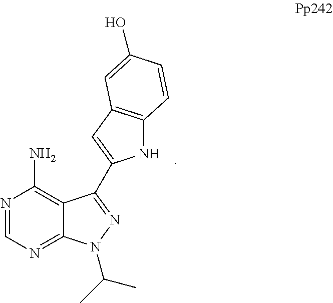 [SMILES: CC(C)n1nc(-c2cc3cc(O)ccc3[nH]2)c2c(N)ncnc21]

In yet another aspect, a method of treating a disease caused by a trypanosomatid parasite is described, comprising administering a therapeutically effective amount of an mTOR and/or PI3K inhibitor compound to a subject in need of treatment, wherein the compound has the structure of Formula (III),

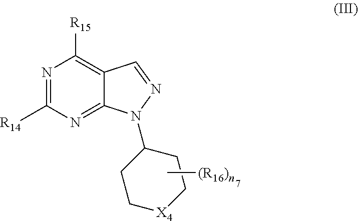

or a pharmaceutically acceptable salt or hydrate thereof,
wherein

R₁₄ is hydrogen, —C₁-C₄ alkyl-NRₐC(O)ORₐ, or

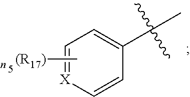

R₁₅ is hydrogen, C₁-C₄ alkyl, aryl, heteroaryl, NRₐR_{b}, or

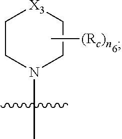

R_{c} and R₁₆ are each independently hydrogen or C₁-C₄ alkyl;

R₁₇ is hydrogen, halogen, OH, CF₃, C₁-C₄ alkyl, ORₐ, OC(O)Rₐ, NRₐR_{b}, NRₐC(O)Rₐ, NRₐC(O)ORₐ, C(O)Rₐ, or C(O)NRₐR_{b};

Rₐ and R_{b} are each independently hydrogen, C₁-C₄ alkyl, benzyl, pyridin-3-ylmethyl, —O—C₁-C₄ alkyl, or —C₁-C₄ alkyl O—C₁-C₄ alkyl;

X is C or N;

X₃ and X₄ are each independently CRₐR_{b}, O, S, NRₐ, NC(O)Rₐ, or NC(O)ORₐ; and

n₅, n₆, and n₇ are each independently 0, 1, 2, 3, or 4.

In some embodiments, X₄ is N-benzyl. In some embodiments, X₄ is N-(pyridin-3-ylmethyl). In some embodiments, X₄ is NC(O)OMe. In some embodiments, X₄ is NH. In some embodiments, R₁₄ is

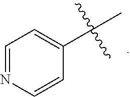 [SMILES: *c1ccncc1]

In some embodiments, R₁₇ is NH₂, OH, NHC(O)OMe, or NHC(O)OC(CH₃)₃. In some embodiments, R₁₅ is H. In some embodiments, R₁₅ is NHCH₂CH₂OCH₂CH₃. In some embodiments, R₁₄ is CH₂CH₂NHC(O)OCH₃. In some embodiments, X₃ is CH₂. In some embodiments, X₃ is O. In some embodiments, the compound is selected from the group consisting of:

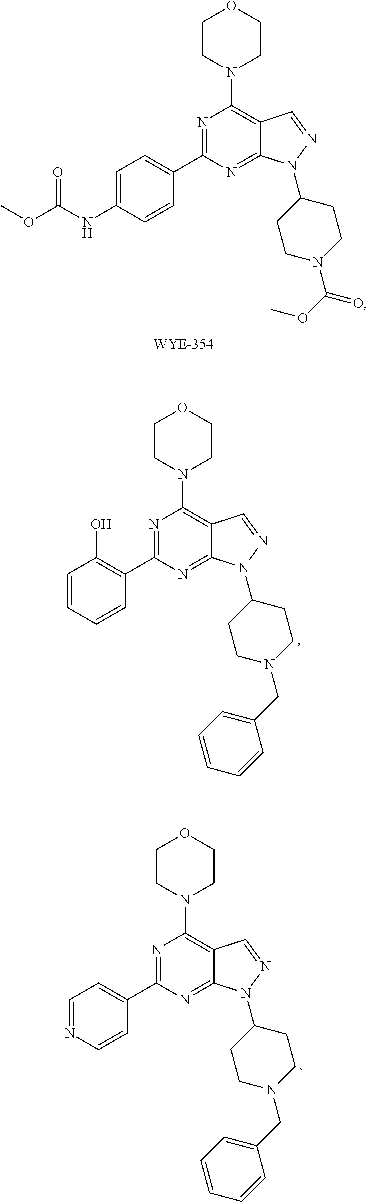 [SMILES: COC(=O)Nc1ccc(-c2nc(N3CCOCC3)c3cnn(C4CCN(C(=O)OC)CC4)c3n2)cc1 ; Oc1ccccc1-c1nc(N2CCOCC2)c2cnn(C3CCN(Cc4ccccc4)CC3)c2n1 ; c1ccc(CN2CCC(n3ncc4c(N5CCOCC5)nc(-c5ccncc5)nc43)CC2)cc1]

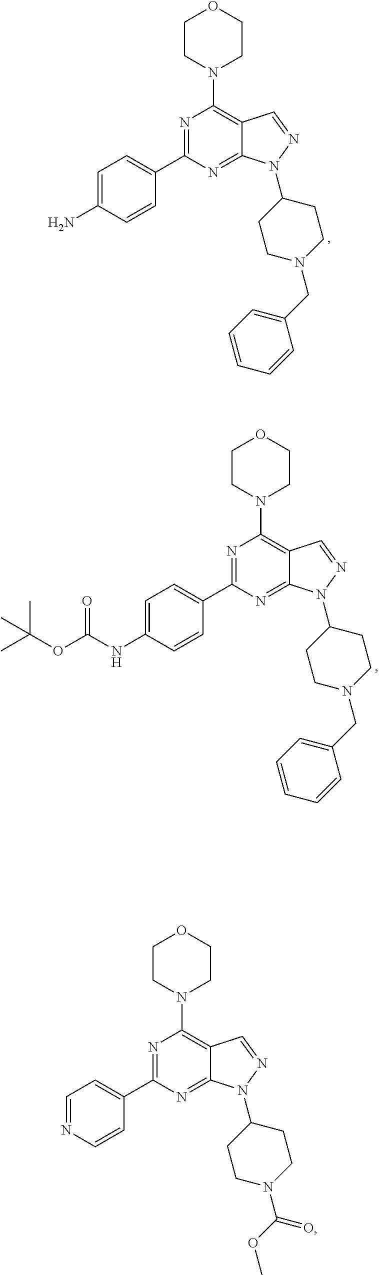 [SMILES: Nc1ccc(-c2nc(N3CCOCC3)c3cnn(C4CCN(Cc5ccccc5)CC4)c3n2)cc1 ; CC(C)(C)OC(=O)Nc1ccc(-c2nc(N3CCOCC3)c3cnn(C4CCN(Cc5ccccc5)CC4)c3n2)cc1 ; COC(=O)N1CCC(n2ncc3c(N4CCOCC4)nc(-c4ccncc4)nc32)CC1]

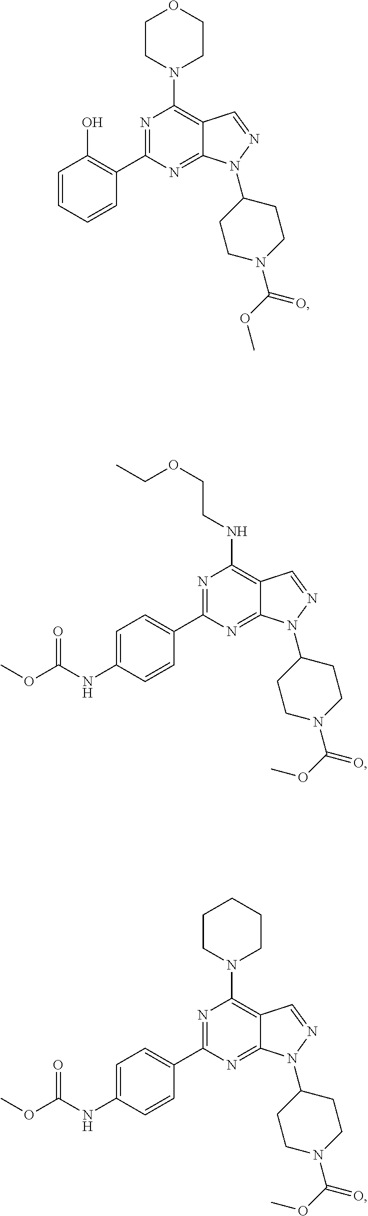 [SMILES: COC(=O)N1CCC(n2ncc3c(N4CCOCC4)nc(-c4ccccc4O)nc32)CC1 ; CCOCCNc1nc(-c2ccc(NC(=O)OC)cc2)nc2c1cnn2C1CCN(C(=O)OC)CC1 ; COC(=O)Nc1ccc(-c2nc(N3CCCCC3)c3cnn(C4CCN(C(=O)OC)CC4)c3n2)cc1]

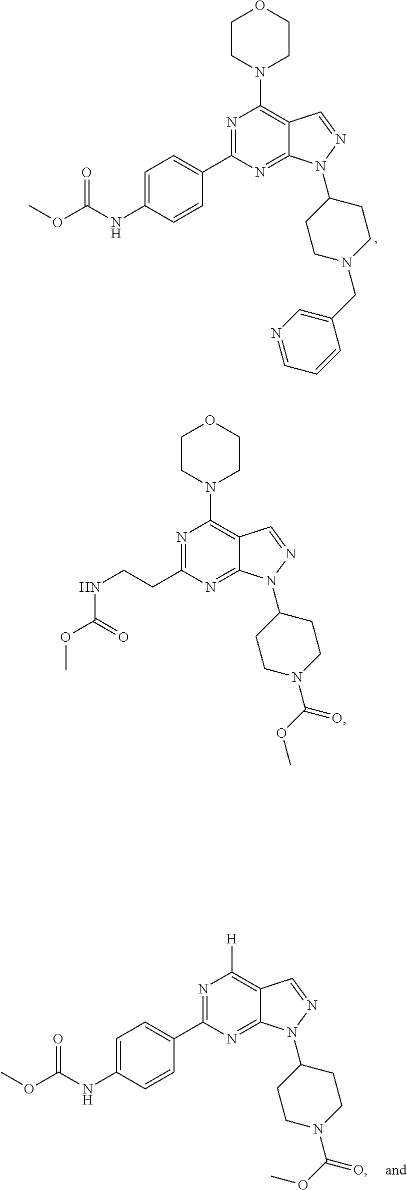

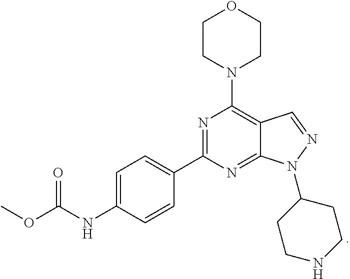 [SMILES: COC(=O)Nc1ccc(-c2nc(N3CCOCC3)c3cnn(C4CCNCC4)c3n2)cc1]

In yet another aspect, a method of treating a disease caused by a trypanosomatid parasite is described, comprising administering a therapeutically effective amount of an mTOR and/or PI3K inhibitor compound to a subject in need of treatment, wherein the compound has the structure of Formula (IV),

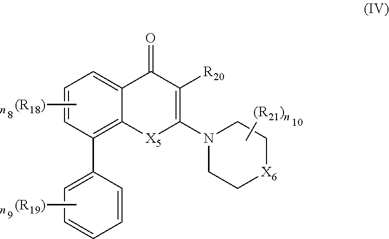

or a pharmaceutically acceptable salt or hydrate thereof,
wherein

R₁₈, R₁₉, and R₂₀ are each independently hydrogen, halogen, OH, CF₃, C₁-C₄ alkyl, ORₐ, OC(O)Rₐ, NRₐR_{b}, NRₐC(O)Rₐ, NRₐC(O)ORₐ, C(O)Rₐ, or C(O)NRₐR_{b};

R₂₁ is hydrogen or C₁-C₄ alkyl;

X₅ and X₆ are each independently CRₐR_{b}, O, S, NRₐ, NC(O)Rₐ, or NC(O)ORₐ;

Rₐ and R_{b} are each independently hydrogen, C₁-C₄ alkyl, benzyl, pyridin-3-ylmethyl, —O—C₁-C₄ alkyl, or —C₁-C₄ alkyl O—C₁-C₄ alkyl; and

n₈, n₉, and n₁₀ are each independently 0, 1, 2, 3, or 4.

In some embodiments, X₅ is O. In some embodiments, X₆ is O. In some embodiments, X₆ is NH. In some embodiments, R₂₀ is H. In some embodiments, the compound is selected from the group consisting of:

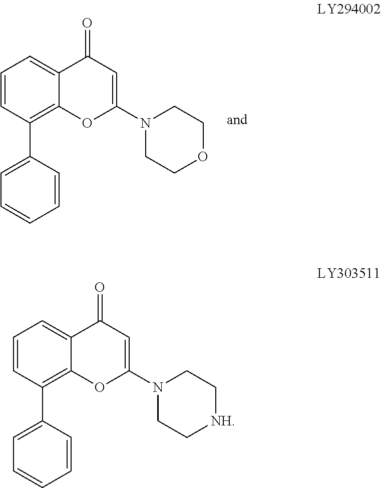 [SMILES: O=c1cc(N2CCOCC2)oc2c(-c3ccccc3)cccc12 ; O=c1cc(N2CCNCC2)oc2c(-c3ccccc3)cccc12]

In yet another aspect, a method of treating a disease caused by a trypanosomatid parasite is described, comprising administering a therapeutically effective amount of an mTOR and/or PI3K inhibitor compound to a subject in need of treatment, wherein the compound has the structure of Formula (V),

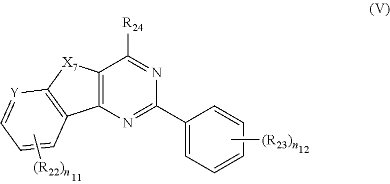

or a pharmaceutically acceptable salt or hydrate thereof,
wherein

R₂₄ is hydrogen, C₁-C₄ alkyl, aryl, heteroaryl, NRₐR_{b},

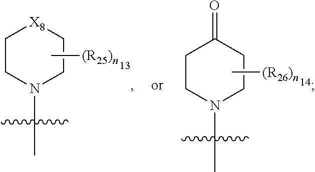

R₂₂ and R₂₃ are each independently hydrogen, halogen, OH, CF₃, C₁-C₄ alkyl, ORₐ, C₁-C₄ alkyl-ORₐ, OC(O)Rₐ, NRₐR_{b}, NRₐC(O)Rₐ, NRₐC(O)ORₐ, C(O)Rₐ, or C(O)NRₐR_{b};

R₂₅ and R₂₆ are each independently hydrogen or C₁-C₄ alkyl;

X₇ and X₈ are each independently CRₐR_{b}, O, S, NRₐ, NC(O)Rₐ, or NC(O)ORₐ;

Rₐ and R_{b} are each independently hydrogen, C₁-C₄ alkyl, benzyl, pyridin-3-ylmethyl, —O—C₁-C₄ alkyl, or —C₁-C₄ alkyl-O—C₁-C₄ alkyl;

Y is C or N; and

n₁₁, n₁₂, n₁₃, and n₁₄ are each independently 0, 1, 2, 3, or 4.

In some embodiments, R₂₄ is H. In some embodiments, R₂₄ is NHCH₂CH₂OCH₂CH₃. In some embodiments, R₂₄ is

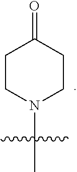 [SMILES: *N1CCC(=O)CC1]

In some embodiments, R₂₄ is

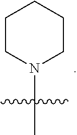 [SMILES: *N1CCCCC1]

In some embodiments, R₂₄ is

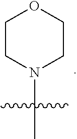 [SMILES: *N1CCOCC1]

In some embodiments, R₂₃ is H. In some embodiments, R₂₃ is ortho-OEt. In some embodiments, R₂₃ is meta-OMe, meta-OH, or meta-OAc. In some embodiments, R₂₃ is para-OMe. In some embodiments, R₂₃ is meta-CH₂CH₂OH. In some embodiments, X₇ is S or O. In some embodiments, Y is N. In some embodiments, Y is C. In some embodiments, the compound is selected from the consisting of:

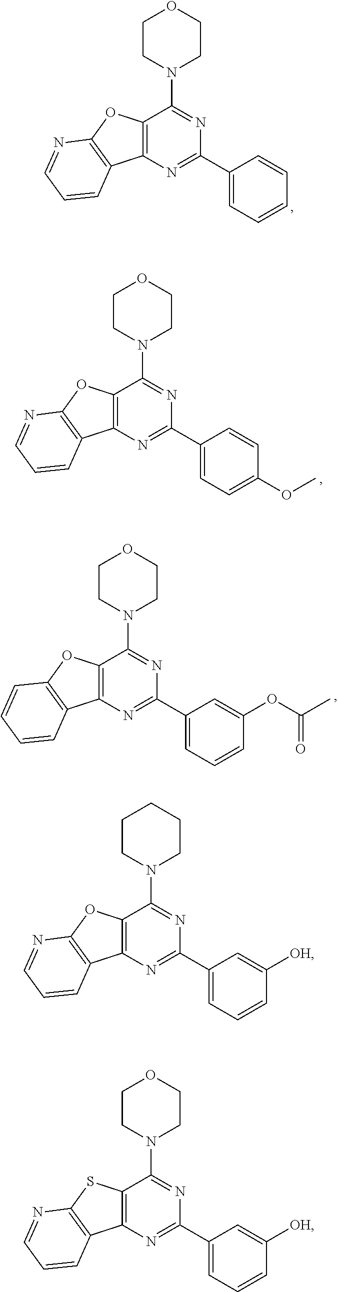 [SMILES: c1ccc(-c2nc(N3CCOCC3)c3oc4ncccc4c3n2)cc1 ; COc1ccc(-c2nc(N3CCOCC3)c3oc4ncccc4c3n2)cc1 ; CC(=O)Oc1cccc(-c2nc(N3CCOCC3)c3oc4ccccc4c3n2)c1 ; Oc1cccc(-c2nc(N3CCCCC3)c3oc4ncccc4c3n2)c1 ; Oc1cccc(-c2nc(N3CCOCC3)c3sc4ncccc4c3n2)c1]

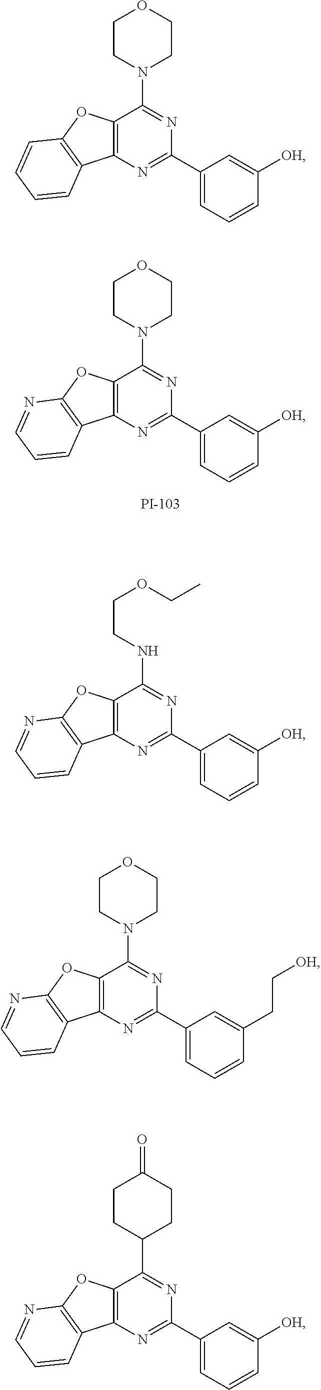 [SMILES: Oc1cccc(-c2nc(N3CCOCC3)c3oc4ccccc4c3n2)c1 ; Oc1cccc(-c2nc(N3CCOCC3)c3oc4ncccc4c3n2)c1 ; CCOCCNc1nc(-c2cccc(O)c2)nc2c1oc1ncccc12 ; OCCc1cccc(-c2nc(N3CCOCC3)c3oc4ncccc4c3n2)c1 ; O=C1CCC(c2nc(-c3cccc(O)c3)nc3c2oc2ncccc23)CC1]

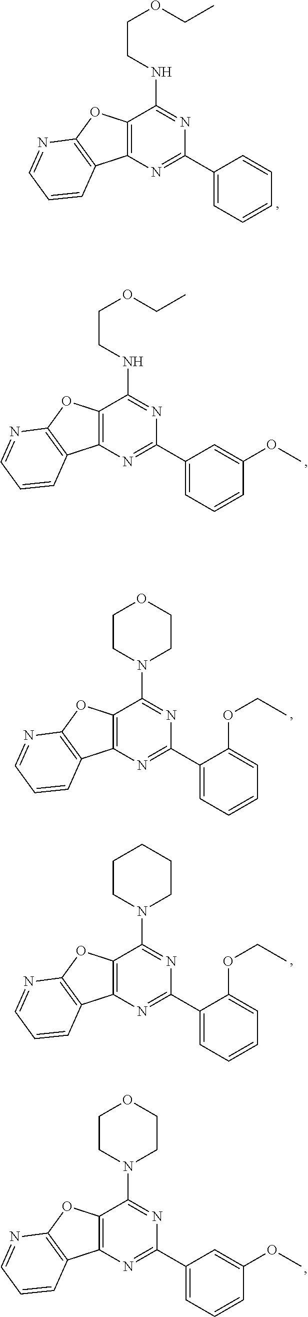 [SMILES: CCOCCNc1nc(-c2ccccc2)nc2c1oc1ncccc12 ; CCOCCNc1nc(-c2cccc(OC)c2)nc2c1oc1ncccc12 ; CCOc1ccccc1-c1nc(N2CCOCC2)c2oc3ncccc3c2n1 ; CCOc1ccccc1-c1nc(N2CCCCC2)c2oc3ncccc3c2n1 ; COc1cccc(-c2nc(N3CCOCC3)c3oc4ncccc4c3n2)c1]

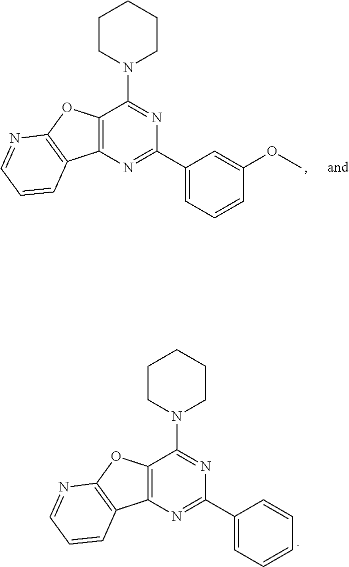 [SMILES: COc1cccc(-c2nc(N3CCCCC3)c3oc4ncccc4c3n2)c1 ; c1ccc(-c2nc(N3CCCCC3)c3oc4ncccc4c3n2)cc1]

In yet another aspect, a method of treating a disease caused by a trypanosomatid parasite is described, comprising administering a therapeutically effective amount of an mTOR and/or PI3K inhibitor compound to a subject in need of treatment, wherein the compound has the structure of Formula (VII),

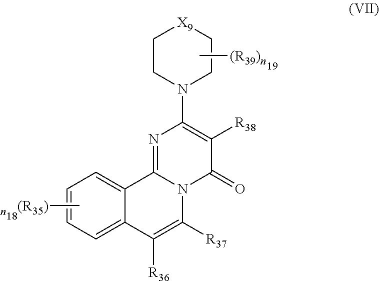

or a pharmaceutically acceptable salt or hydrate thereof,
wherein

R₃₅, R₃₆, R₃₇, and R₃₈ are each independently hydrogen, halogen, OH, CF₃, C₁-C₄ alkyl, ORₐ, OC(O)Rₐ, NRₐR_{b}, NRₐC(O)Rₐ, NRₐC(O)ORₐ, C(O)Rₐ, or C(O)NRₐR_{b};

R₃₉ is hydrogen or C₁-C₄ alkyl;

X₉ is CRₐR_{b}, O, S, NRₐ, NC(O)Rₐ, or NC(O)ORₐ;

Rₐ and R_{b} are each independently hydrogen, C₁-C₄ alkyl, benzyl, pyridin-3-ylmethyl, —O—C₁-C₄ alkyl, or —C₁-C₄ alkyl O—C₁-C₄ alkyl; and

n₁₈ and n₁₉ are each independently 0, 1, 2, 3, or 4.

In some embodiments, R₃₅ is H or OMe. In some embodiments, X₉ is O. In some embodiments, the compound is

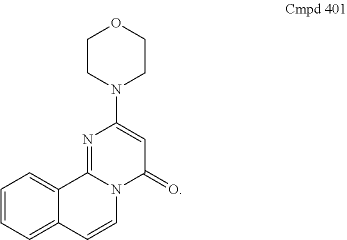 [SMILES: O=c1cc(N2CCOCC2)nc2c3ccccc3ccn12]

In yet another aspect, use of the compound of Formulas (I), (II), (III), (IV), (V), (VI), and (VII) as described above or a pharmaceutically acceptable salt or hydrate thereof in the manufacture of medicament for the treatment of a disease caused by a trypanosomatid parasite is disclosed.

In some embodiments, the disease is selected from the group consisting of Human African Trypanosomiasis, leishmaniasis, and Chagas Disease. In some embodiments, the Human African Trypanosomiasis is caused by Trypanosoma brucei. In some embodiments the leishmaniasis is caused by Leishmania sp. In some embodiments, leishmaniasis is visceral or cutaneous leishmaniasis. In some embodiments, the Chagas Disease is caused by Trypanosoma cruzi. In some embodiments, the disease is nagana, i.e., T. Brucei ssp. in cattle. In some embodiments, the subject is a mammal. In some embodiments, the subject is a livestock or domestic animal. In some embodiments, the mammal is a human.

### Methods of Preparation

Following are general synthetic schemes for manufacturing compounds for use in the disclosed methods. These schemes are illustrative and are not meant to limit the possible techniques one skilled in the art any use to manufacture compounds disclosed herein. Different methods will be evident to those skilled in the art. Additionally, the various steps in the synthesis may be performed in an alternate sequence or order to give the desired compound(s). All documents cited herein are incorporated herein by reference in their entirety. For example, the following reactions are illustrations but not limitations of the preparation of some of the starting materials and examples used herein.

Schemes 1-7 describe various methods for the synthesis of compounds of the present invention. Various modifications to these methods may be envisioned by those skilled in the art to achieve similar results to that of the inventors given below.

Compound of Formula (VI) may be prepared as shown in Scheme 1.

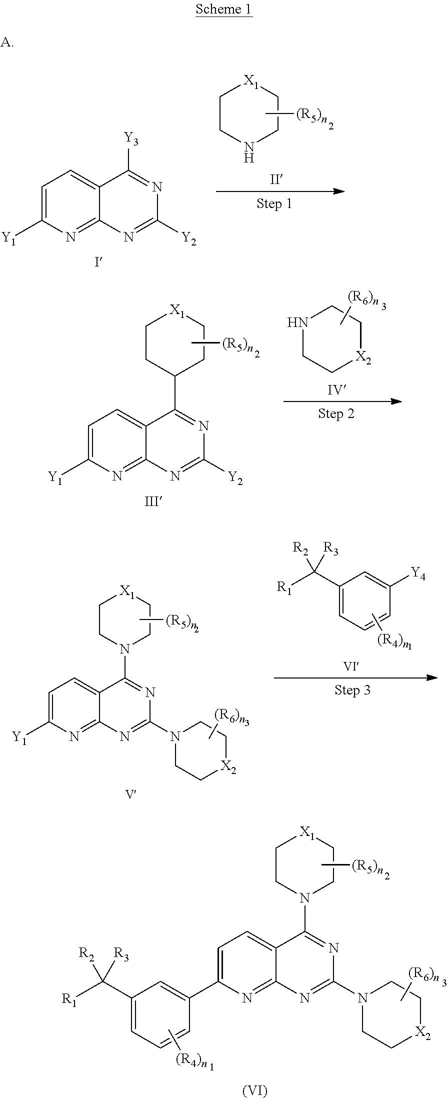

Pyrido[2,3-d]pyrimidine I′, amines II′ and IV′, and substituted benzene VI′ may be readily commercially available or be prepared by methods known to one of ordinary skill in the art.

### Step 1

Pyrido[2,3-d]pyrimidine I′ may react with amine II′ to afford substituted pyridopyrimidine III′. Y₃ is halogen, OTf, OTs, or other leaving groups well known in the art. Step 1 may be carried out in the present of one or more suitable bases, which include, but are not limited to, triethylamine and diisopropylethylamine. Optionally, a catalyst such as Pd(0) can be used to catalyze this reaction. Suitable solvent for this reaction includes methylene chloride, Dimethylacetamide, DMF, methanol, and acetonitrile.

### Step 2

Pyridopyrimidine III′ may react with amine IV′ to afford substituted pyridopyrimidine V′. Y₂ is halogen, OTf, OTs, or other leaving groups well known in the art. Step 2 may be carried out in the present of one or more suitable bases, which include, but are not limited to, triethylamine, and diisopropylethylamine. Optionally, a catalyst such as Pd(0) can be used to catalyze this reaction. Suitable solvent for this reaction includes methylene chloride, Dimethylacetamide, DMF, methanol, and acetonitrile.

### Step 3

Pyridopyrimidine V′ may react with substituted benzene VI′ to afford a compound of Formula (VI). Y₁ and Y₄ are each independently halogen, OTf, OTs, other leaving groups well known in the art, B(OH)₂, or other boron agents, with the proviso that Y₁ and Y₄ are not both boron agents. Step 3 may be carried out in the present of one or more suitable bases, which include, but are not limited to, triethylamine, diisopropylethylamine, NaHCO₃, and Na₂CO₃. Optionally, a catalyst such as Pd(0) can be used to catalyze this reaction. Suitable solvent for this reaction includes methylene chloride, Dimethylacetamide, DMF, methanol, and acetonitrile.

Compound of Formula (II) may be prepared as shown in Scheme 2.

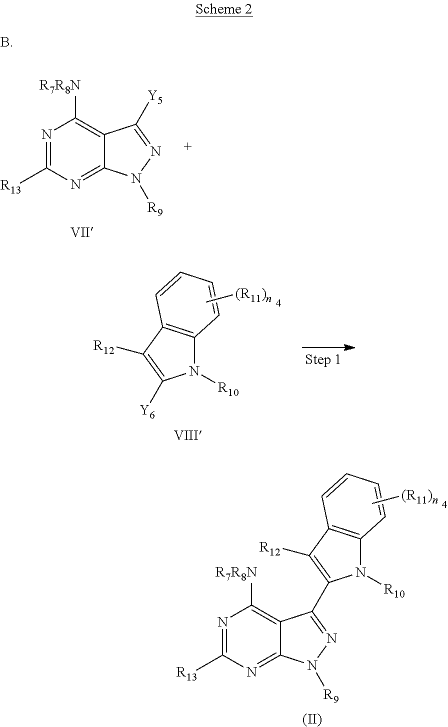

Substituted 1H-pyrazolo[3,4-d]pyrimidine VII′ and indole VIII′ may be readily commercially available or be prepared by methods known to one of ordinary skill in the art.

### Step 1

Substituted 1H-pyrazolo[3,4-d]pyrimidine VII′ may react with optionally substituted indole VIII′ to afford a compound of Formula (II). Y₅ and Y₆ are each independently halogen, OTf, OTs, other leaving groups well known in the art, B(OH)₂, or other boron agents, with the proviso that Y₅ and Y₆ are not both boron agents. Step 1 may be carried out in the present of one or more suitable bases, which include, but are not limited to, triethylamine, diisopropylethylamine, NaHCO₃, and Na₂CO₃. Optionally, a catalyst such as Pd(0) can be used to catalyze this reaction. Suitable solvent for this reaction includes methylene chloride, Dimethylacetamide, DMF, methanol, and acetonitrile.

Compound of Formula (III) may be prepared as shown in Scheme 3.

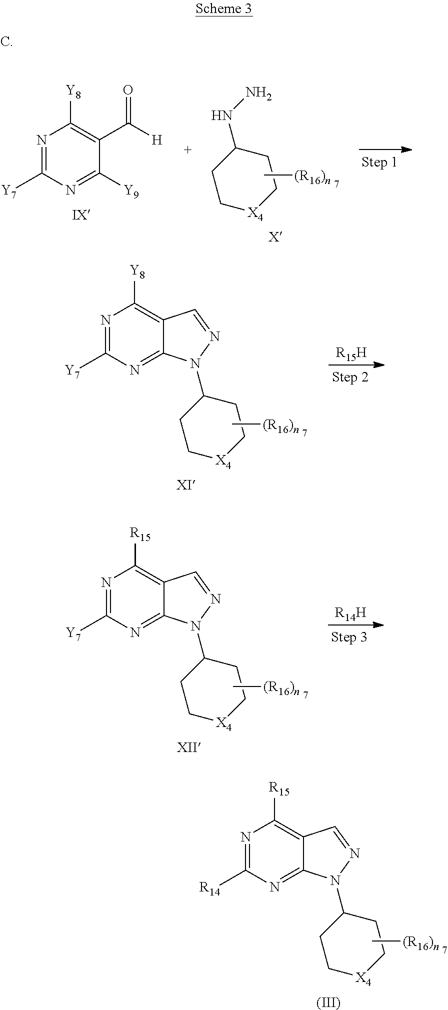

Aldehyde IX′, hydrazine X′, R₁₅H, and R₁₄H may be readily commercially available or be prepared by methods known to one of ordinary skill in the art.

### Step 1

Optionally substituted aldehyde IX′ may undergo condensation reaction with hydrazine X′ to afford substituted pyrazolopyrimidine XI′. Y₉ is a halogen, OTf, OTs, or other leaving groups well known in the art. Step 1 may be carried out in the present of one or more suitable bases, which include, but are not limited to, triethylamine and diisopropylethylamine. Suitable solvent for this reaction includes methylene chloride, Dimethylacetamide, DMF, methanol, and acetonitrile.

### Step 2

Pyrazolopyrimidine XI′ may react with R₁₅H to afford pyrazolopyrimidine XII′. Y₈ is a halogen, OTf, OTs, or other leaving groups well known in the art. Step 2 may be carried out in the present of one or more suitable bases, which include, but are not limited to, triethylamine and diisopropylethylamine. Optionally, a catalyst such as Pd(0) can be used to catalyze this reaction. Suitable solvent for this reaction includes methylene chloride, Dimethylacetamide, DMF, methanol, and acetonitrile.

### Step 3

Pyrazolopyrimidine XII′ may react with R₁₄H to afford a compound of Formula (III). Y₇ is halogen, OTf, OTs, other leaving groups well known in the art, B(OH)₂, or other boron agents. Step 3 may be carried out in the present of one or more suitable bases, which include, but are not limited to, triethylamine, diisopropylethylamine, NaHCO₃, and Na₂CO₃. Optionally, a catalyst such as Pd(0) can be used to catalyze this reaction. Suitable solvent for this reaction includes methylene chloride, Dimethylacetamide, DMF, methanol, and acetonitrile.

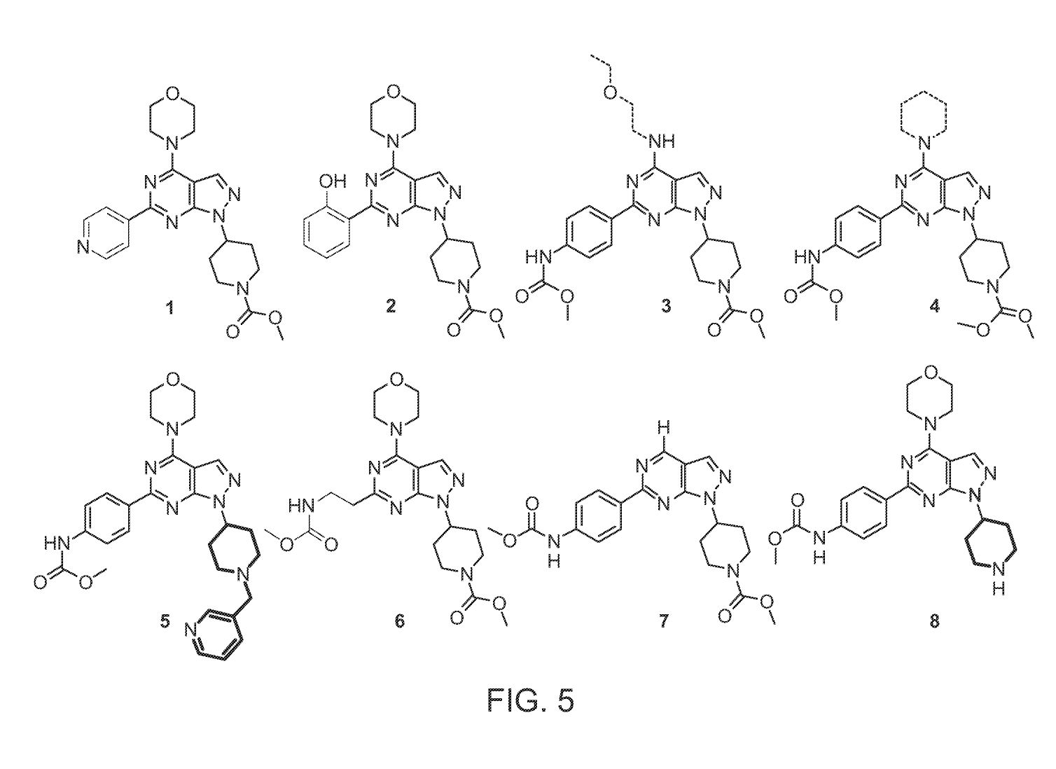

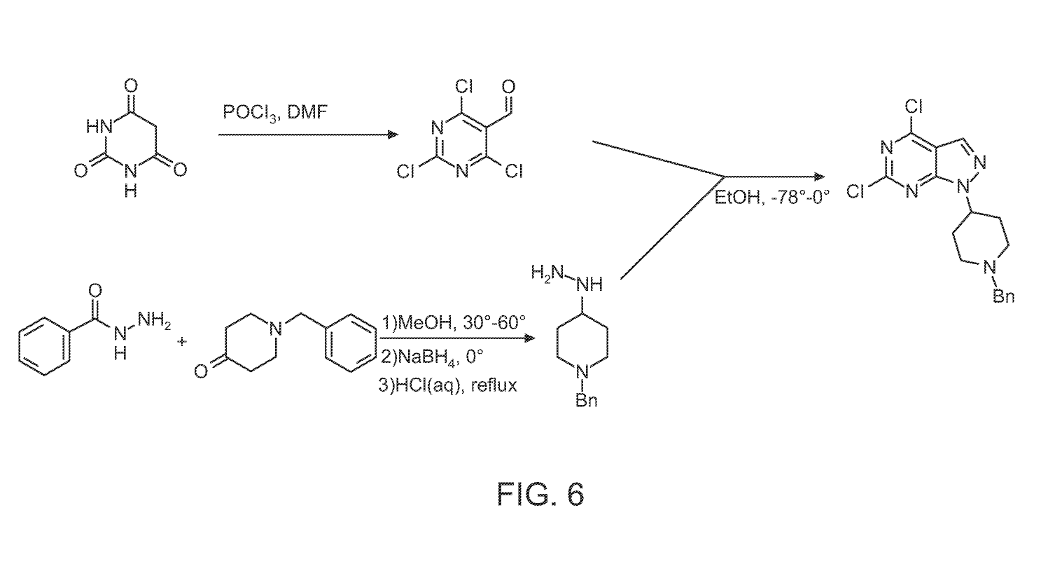

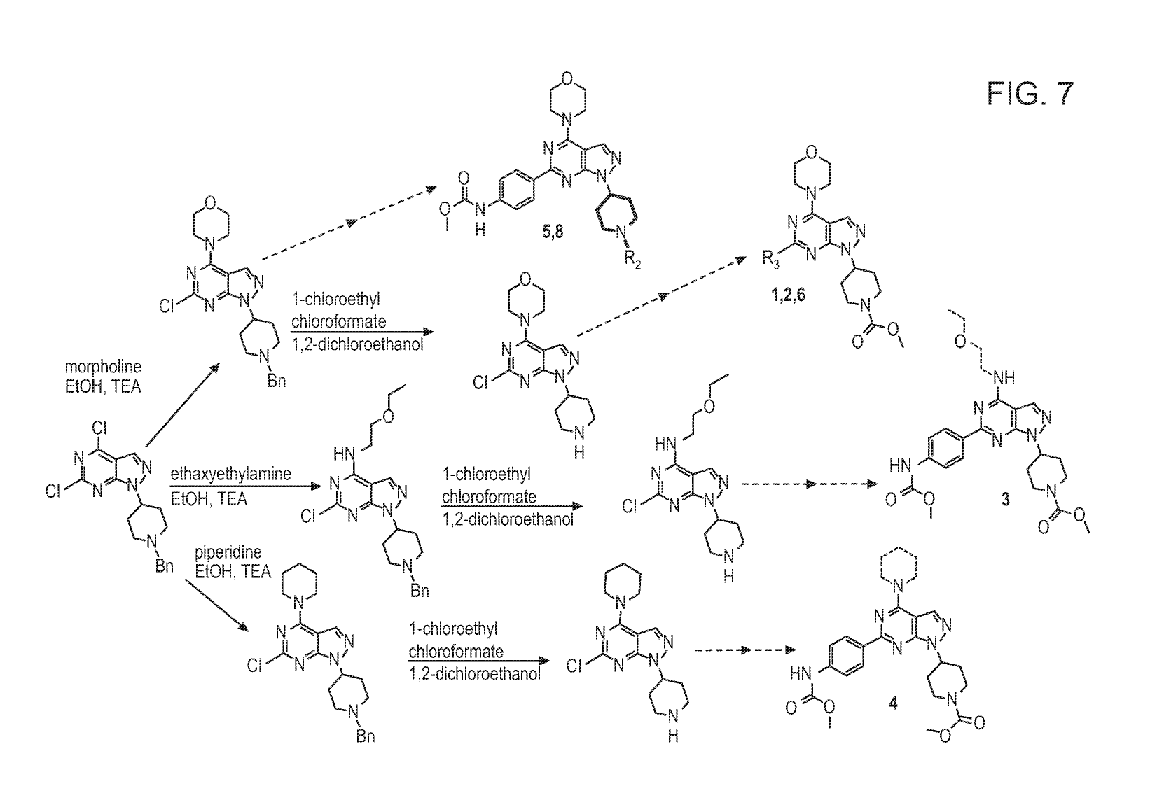

In some specific embodiments, the compound of Formula (III) is selected from compounds 1-8 as shown in FIG. 5. In accordance with Scheme 3, compounds 1-6 and 8 may be prepared following the synthetic methods shown in FIGS. 6-7.

Compound of Formula (IV) may be prepared as shown in Scheme 4.

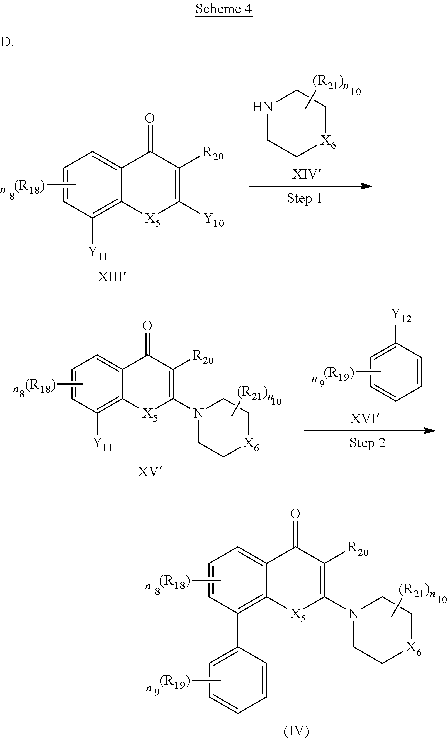

Bicyclic compound XIII′, amine XIV′, and benzene XVI′ may be readily commercially available or be prepared by methods known to one of ordinary skill in the art.

### Step 1

Bicyclic compound XIII′ may react with amine XIV′ to afford compound XV′. Y₁₀ is a halogen, OTf, OTs, or other leaving groups well known in the art. Step 1 may be carried out in the present of one or more suitable bases, which include, but are not limited to, triethylamine and diisopropylethylamine. Optionally, a catalyst such as Pd(0) can be used to catalyze this reaction. Suitable solvent for this reaction includes methylene chloride, Dimethylacetamide, DMF, methanol, and acetonitrile.

### Step 2

Compound XV′ may react with optionally substituted benzene XVI′ to afford a compound of Formula (IV). Y₁₁ and Y₁₂ are halogen, OTf, OTs, other leaving groups well known in the art, B(OH)₂, or other boron agents, with the proviso that Y₁₁ and Y₁₂ are not both boron agents. Step 2 may be carried out in the present of one or more suitable bases, which include, but are not limited to, triethylamine and diisopropylethylamine. Optionally, a catalyst such as Pd(0) can be used to catalyze this reaction. Suitable solvent for this reaction includes methylene chloride, Dimethylacetamide, DMF, methanol, and acetonitrile.

Compound of Formula (V) may be prepared as shown in Scheme 5.

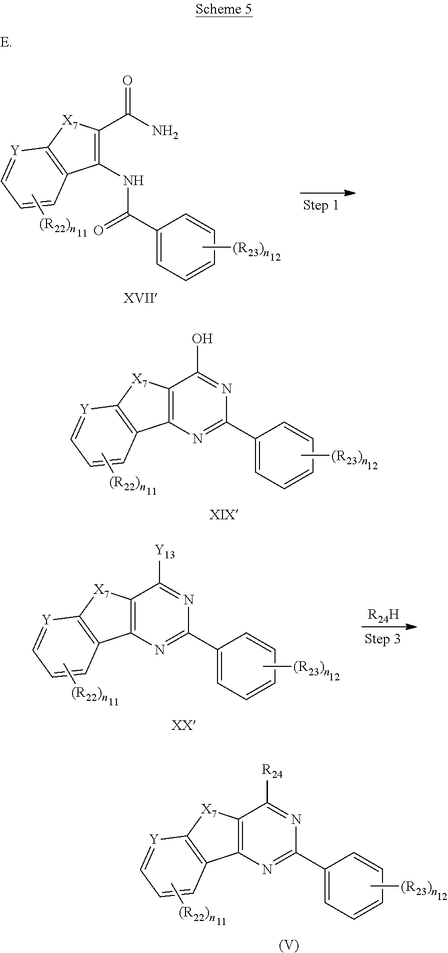

Bicyclic compound XVII′ and R₂₄H may be readily commercially available or be prepared by methods known to one of ordinary skill in the art.

### Step 1

Bicyclic compound XVII′ may undergo cyclization reaction to afford compound XIX′. Optionally, an oxidizing agent is used in this reaction. Non-limiting examples of the oxidizing agents include peracids, permanganate, and hydrogen peroxide. Suitable solvent for this reaction includes methylene chloride, Dimethylacetamide, DMF, methanol, and acetonitrile.

### Step 2

Compound XIX′ may react with halogenation agents or esterification agents known in the art to afford compound XX′. Y₁₃ is halogen, OTf, OTs, or other leaving groups well known in the art. Step 2 may be carried out in the present of one or more suitable bases, which include, but are not limited to, triethylamine and diisopropylethylamine. Suitable solvent for this reaction includes methylene chloride, Dimethylacetamide, DMF, methanol, and acetonitrile.

### Step 3

Compound XX′ may react with R₂₄H to afford a compound of Formula (V). Step 3 may be carried out in the present of one or more suitable bases, which include, but are not limited to, triethylamine and diisopropylethylamine. Optionally, a catalyst such as Pd(0) can be used to catalyze this reaction. Suitable solvent for this reaction includes methylene chloride, Dimethylacetamide, DMF, methanol, and acetonitrile.

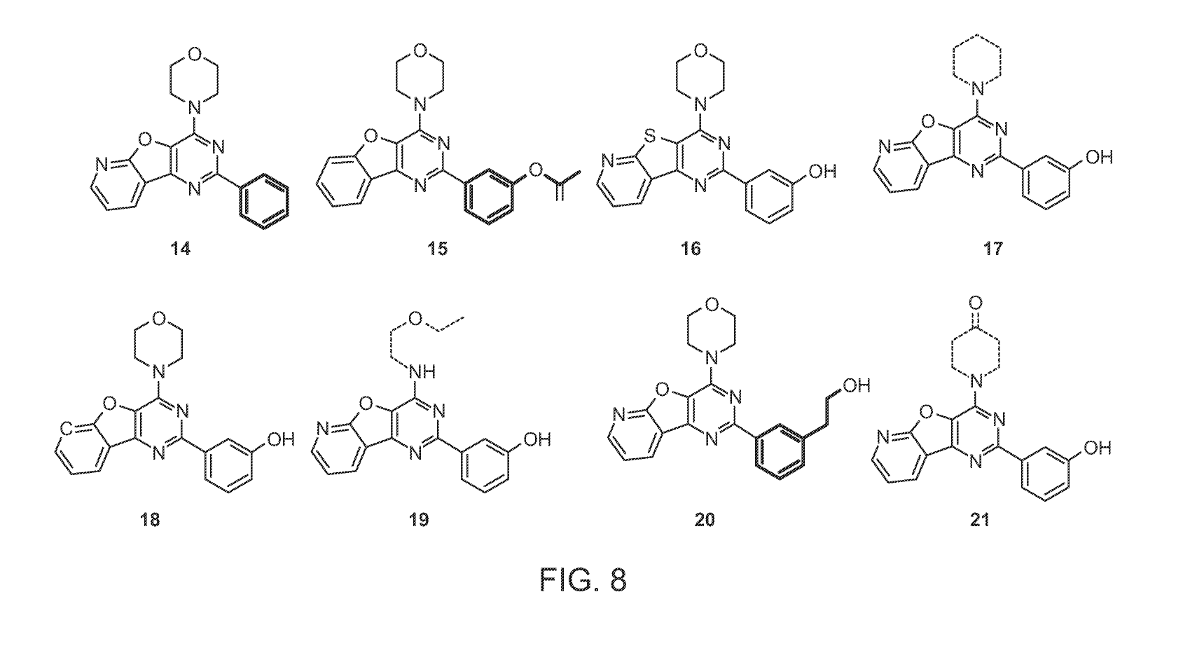

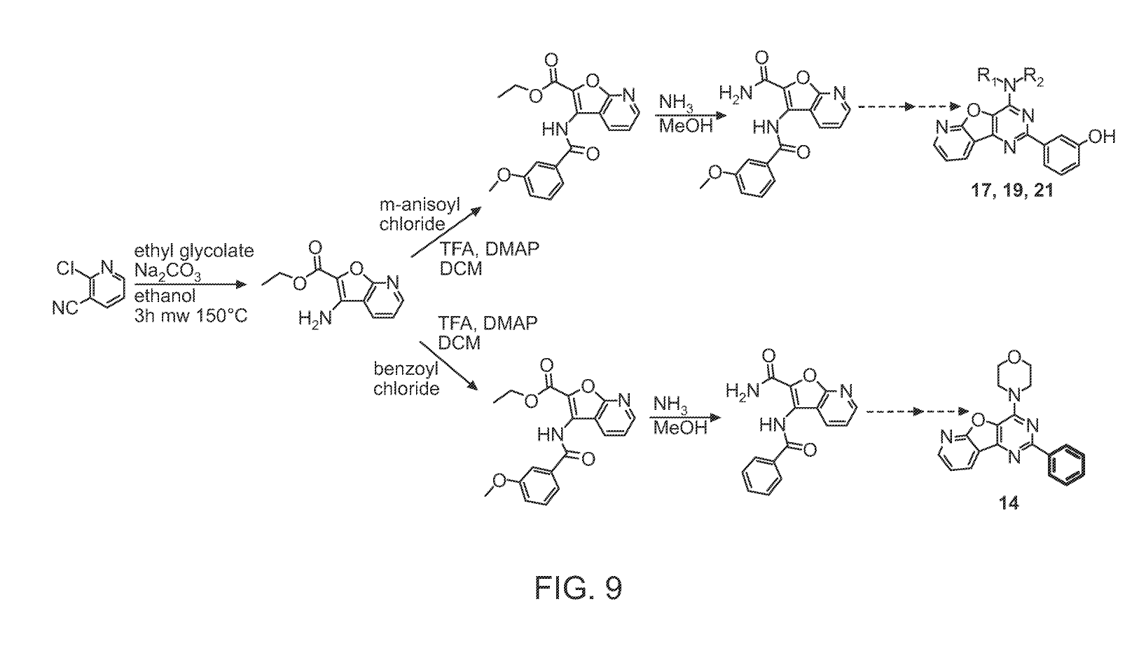

In some specific embodiments, the compound of Formula (V) is selected from compounds 14-21 as shown in FIG. 8. In accordance with Scheme 5, compounds 14, 17, 19, and 21 may be prepared following the synthetic methods shown in FIG. 9.

Compound of Formula (I) may be prepared as shown in Scheme 6.

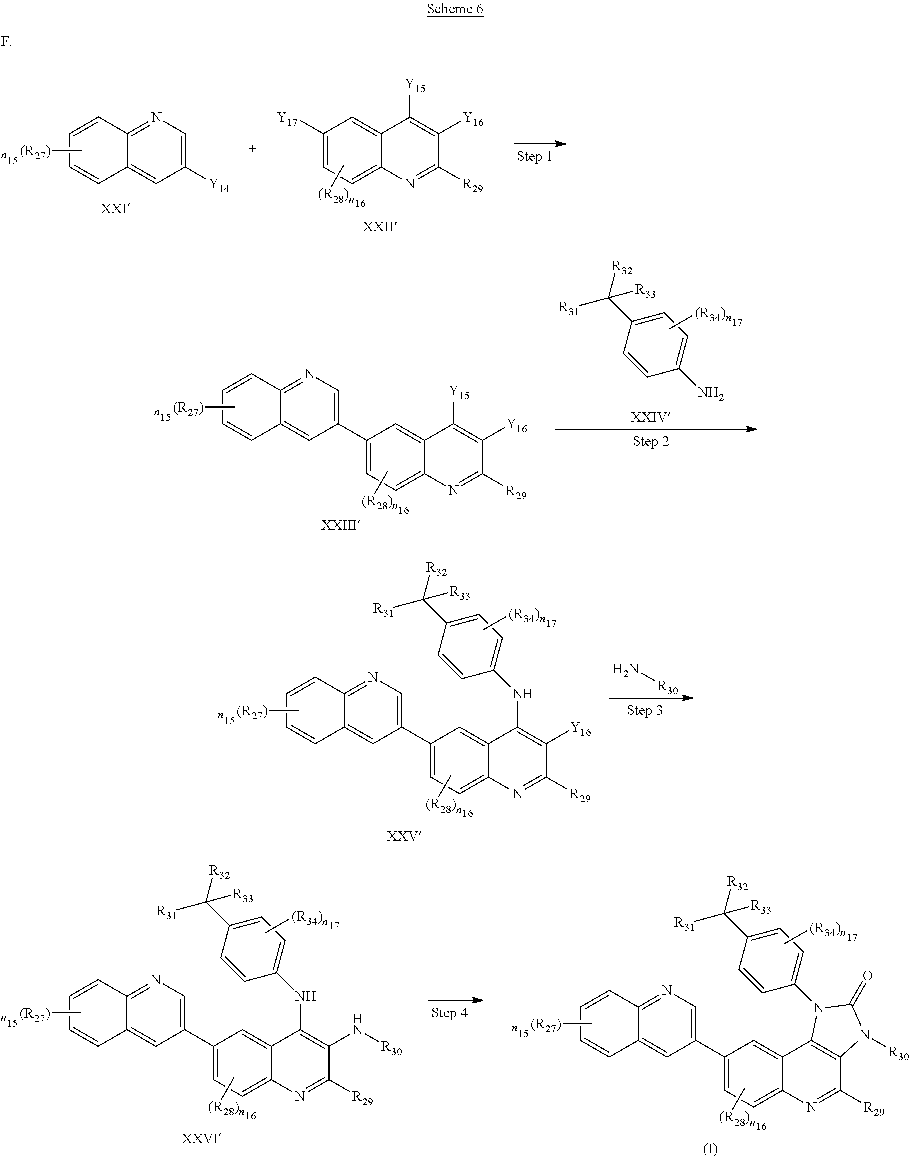

Quinoline compound XXI′, quinoline compound XXII′, aniline XXIV′, and R₃₀NH₂ may be readily commercially available or be prepared by methods known to one of ordinary skill in the art.

### Step 1

Quinoline compound XXI′ may be coupled with quinoline compound XXII′ to afford compound XXIII′. Y₁₄ and Y₁₇ are halogen, OTf, OTs, other leaving groups well known in the art, B(OH)₂, or other boron agents, with the proviso that Y₁₄ and Y₁₇ are not both boron agents. Step 1 may be carried out in the present of one or more suitable bases, which include, but are not limited to, triethylamine and diisopropylethylamine. Optionally, a catalyst such as Pd(0) can be used to catalyze this reaction. Suitable solvent for this reaction includes methylene chloride, Dimethylacetamide, DMF, methanol, and acetonitrile.

### Step 2

Compound XXIII′ may react with aniline XXIV′ to afford compound XXV′. Y₁₅ is halogen, OTf, OTs, or other leaving groups well known in the art. Step 2 may be carried out in the present of one or more suitable bases, which include, but are not limited to, triethylamine and diisopropylethylamine. Optionally, a catalyst such as Pd(0) can be used to catalyze this reaction. Suitable solvent for this reaction includes methylene chloride, Dimethylacetamide, DMF, methanol, and acetonitrile.

### Step 3

Compound XXV′ may react with R₃OH to afford compound XXVI′. Y₁₆ is halogen, OTf, OTs, or other leaving groups well known in the art. Step 3 may be carried out in the present of one or more suitable bases, which include, but are not limited to, triethylamine and diisopropylethylamine. Optionally, a catalyst such as Pd(0) can be used to catalyze this reaction. Suitable solvent for this reaction includes methylene chloride, Dimethylacetamide, DMF, methanol, and acetonitrile.

### Step 4

Compound XXVI′ may be cyclized to form a compound of Formula (I). A cyclizing agent is used in this reaction. Non-limiting examples of cyclizing agents include C(O)Cl₂. Step 4 may be carried out in the present of one or more suitable bases, which include, but are not limited to, triethylamine and diisopropylethylamine. Suitable solvent for this reaction includes methylene chloride, Dimethylacetamide, DMF, methanol, and acetonitrile.

Compound of Formula (VII) may be prepared as shown in Scheme 7.

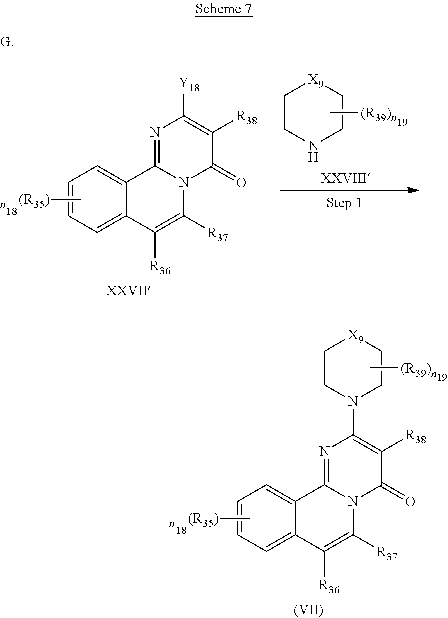

Compound XXVII′ and amine XXVIII′ may be readily commercially available or be prepared by methods known to one of ordinary skill in the art.

### Step 1

Compound XXVII′ may be coupled with amine XXVIII′ to afford a compound of Formula (VII). Y₁₈ is halogen, OTf, OTs, or other leaving groups well known in the art. Step 1 may be carried out in the present of one or more suitable bases, which include, but are not limited to, triethylamine and diisopropylethylamine. Optionally, a catalyst such as Pd(0) can be used to catalyze this reaction. Suitable solvent for this reaction includes methylene chloride, Dimethylacetamide, DMF, methanol, and acetonitrile.

### Pharmaceutical Compositions

In another aspect, the disclosure provides pharmaceutically acceptable preparations comprising a therapeutically effective amount of one or more of the compounds disclosed herein, formulated together with one or more pharmaceutically acceptable carriers (additives) and/or diluents. The pharmaceutical compositions of the present disclosure can be specially formulated for administration in solid or liquid form, including but not limited to those adapted for the following: (1) oral administration, for example, drenches (aqueous or non-aqueous solutions or suspensions), tablets, boluses, powders, granules, pastes for application to the tongue; or (2) parenteral administration, for example, by subcutaneous, intramuscular or intravenous injection as, for example, a sterile solution or suspension.

This invention provides a pharmaceutical composition comprising at least one of the compounds as described herein or a pharmaceutically-acceptable salt or solvate thereof, and a pharmaceutically-acceptable carrier.

In yet another aspect, a pharmaceutical composition is described, comprising at least one a compound of Formula (I), (II), (III), (IV), (V), (VI), or (VII) as described herein or a pharmaceutically acceptable salt thereof, and a pharmaceutically-acceptable carrier or diluent.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as butylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

As set out above, certain embodiments of the present pharmaceutical agents may be provided in the form of pharmaceutically-acceptable salts. The term “pharmaceutically-acceptable salt”, in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al., (1977) “Pharmaceutical Salts”, J. Pharm. Sci. 66:1-19.)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, butionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra.)

Wetting agents, emulsifiers and lubricants, such as sodium lauryl sulfate, magnesium stearate, and polyethylene oxide-polybutylene oxide copolymer as well as coloring agents, release agents, coating agents, sweetening, flavoring and perfuming agents, preservatives and antioxidants can also be present in the compositions.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of 100%, this amount will range from about 1% to about 99% of active ingredient, preferably from about 5% to about 70%, most preferably from about 10% to about 30%.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polybutylene oxide copolymer; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxybutylmethyl cellulose), lubricant, inert diluent, preservative, disintegrant (for example, sodium starch glycolate or cross-linked sodium carboxymethyl cellulose), surface-active or dispersing agent. Molded tablets, may be, made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxybutylmethyl cellulose in varying butortions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if apbutriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isobutyl alcohol, ethyl carbonate, ethyl acetate, benzyl alcohol, benzyl benzoate, butylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Additionally, cyclodextrins, e.g., hydroxybutyl-.beta.-cyclodextrin, may be used to solubilize compounds.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active pharmaceutical agents of the invention.

Formulations of the present invention which are suitable for vaginal administration also include pessaries, tampons, creams, gels, pastes, foams or spray formulations containing such carriers as are known in the art to be apbutriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or butellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary butellants, such as chlorofluorohydrocarbons and volatile unsubstituted hydrocarbons, such as butane and butane.

Transdermal patches have the added advantage of providing controlled delivery of a compound of the present invention to the body. Such dosage forms can be made by dissolving, or dispersing the pharmaceutical agents in the buter medium. Absorption enhancers can also be used to increase the flux of the pharmaceutical agents of the invention across the skin. The rate of such flux can be controlled, by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle. One strategy for depot injections includes the use of polyethylene oxide-polybutylene oxide copolymers wherein the vehicle is fluid at room temperature and solidifies at body temperature.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly (orthoesters) and poly (anhydrides). Depot injectable formulations are also prepared by entrapping the drug in liposomes or microemulsions, which are compatible with body tissue.

When the compounds of the present invention are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier.

The compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound of the present invention may be administered concurrently with another anti-inflammatory or immunesupressant agent); such as but not limited to NSAIDS, DMARDS, Steroids, or biologics such as antibody therapies) or they may achieve different effects (e.g., control of any adverse effects).

The compounds of the invention may be administered intravenously, intramuscularly, intraperitoneally, subcutaneously, topically, orally, or by other acceptable means. The compounds may be used to treat arthritic conditions in mammals (i.e., humans, livestock, and domestic animals), birds, lizards, and any other organism, which can tolerate the compounds.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### Administration to a Subject

Some aspects of the invention involve administering an effective amount of a composition to a subject to achieve a specific outcome. The small molecule compositions useful according to the methods of the present invention thus can be formulated in any manner suitable for pharmaceutical use.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the compound can be administered to a subject by any mode allowing the compound to be taken up by the appropriate target cells. “Administering” the pharmaceutical composition of the present invention can be accomplished by any means known to the skilled artisan. Specific routes of administration include but are not limited to oral, transdermal (e.g., via a patch), parenteral injection (subcutaneous, intradermal, intramuscular, intravenous, intraperitoneal, intrathecal, etc.), or mucosal (intranasal, intratracheal, inhalation, intrarectal, intravaginal, etc.). An injection can be in a bolus or a continuous infusion.

For example the pharmaceutical compositions according to the invention are often administered by intravenous, intramuscular, or other parenteral means, or by biolistic “gene-gun” application to the epidermis. They can also be administered by intranasal application, inhalation, topically, orally, or as implants, and even rectal or vaginal use is possible. Suitable liquid or solid pharmaceutical preparation forms are, for example, aqueous or saline solutions for injection or inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer R (1990) Science 249:1527-33, which is incorporated herein by reference.

The concentration of compounds included in compositions used in the methods of the invention can range from about 1 nM to about 100 μM. Effective doses are believed to range from about 10 picomole/kg to about 100 micromole/kg.

The pharmaceutical compositions are preferably prepared and administered in dose units. Liquid dose units are vials or ampoules for injection or other parenteral administration. Solid dose units are tablets, capsules, powders, and suppositories. For treatment of a patient, depending on activity of the compound, manner of administration, purpose of the administration (i.e., prophylactic or therapeutic), nature and severity of the disorder, age and body weight of the patient, different doses may be necessary. The administration of a given dose can be carried out both by single administration in the form of an individual dose unit or else several smaller dose units. Repeated and multiple administration of doses at specific intervals of days, weeks, or months apart are also contemplated by the invention.

The compositions can be administered per se (neat) or in the form of a pharmaceutically acceptable salt. When used in medicine the salts should be pharmaceutically acceptable, but non-pharmaceutically acceptable salts can conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Compositions suitable for parenteral administration conveniently include sterile aqueous preparations, which can be isotonic with the blood of the recipient. Among the acceptable vehicles and solvents are water, Ringer's solution, phosphate buffered saline, and isotonic sodium chloride solution. In addition, sterile, fixed oils are conventionally employed as a solvent or suspending medium. For this purpose any bland fixed mineral or non-mineral oil may be employed including synthetic mono- or diglycerides. In addition, fatty acids such as oleic acid find use in the preparation of injectables. Carrier formulations suitable for subcutaneous, intramuscular, intraperitoneal, intravenous, etc. administrations can be found in Remington's Pharmaceutical Sciences, Mack Publishing Company, Easton, Pa.

The compounds useful in the invention can be delivered in mixtures of more than two such compounds. A mixture can further include one or more adjuvants in addition to the combination of compounds.

A variety of administration routes is available. The particular mode selected will depend, of course, upon the particular compound selected, the age and general health status of the subject, the particular condition being treated, and the dosage required for therapeutic efficacy. The methods of this invention, generally speaking, can be practiced using any mode of administration that is medically acceptable, meaning any mode that produces effective levels of response without causing clinically unacceptable adverse effects. Preferred modes of administration are discussed above.

The compositions can conveniently be presented in unit dosage form and can be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product.

Other delivery systems can include time-release, delayed release or sustained release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

### EQUIVALENTS

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification and guidance which can be adapted to the practice of this invention in its various embodiments and equivalents thereof.

### Examples

Those skilled in the art will recognize, or be able to ascertain, using no more than routine experimentation, numerous equivalents to the specific substances and procedures described herein. Such equivalents are intended to be encompassed in the scope of the claims that follow the examples below.

The Examples described below test the potency of established phosphoinosityl-3-kinase (PI3K) inhibitors against three trypanosomatid parasites: Trypanosoma brucei, T. cruzi, and Leishmania sp., which are the causative agents for African sleeping sickness, Chagas disease, and leishmaniases, respectively. Among these inhibitors is a potent clinical candidate against cancer, NVP-BEZ235, which was demonstrated to be a highly potent trypanocide against parasite cultures, and in a mouse model of T. brucei infection. Additionally, the examples describe observations of these inhibitors' effects on parasite growth and other cellular characteristics.

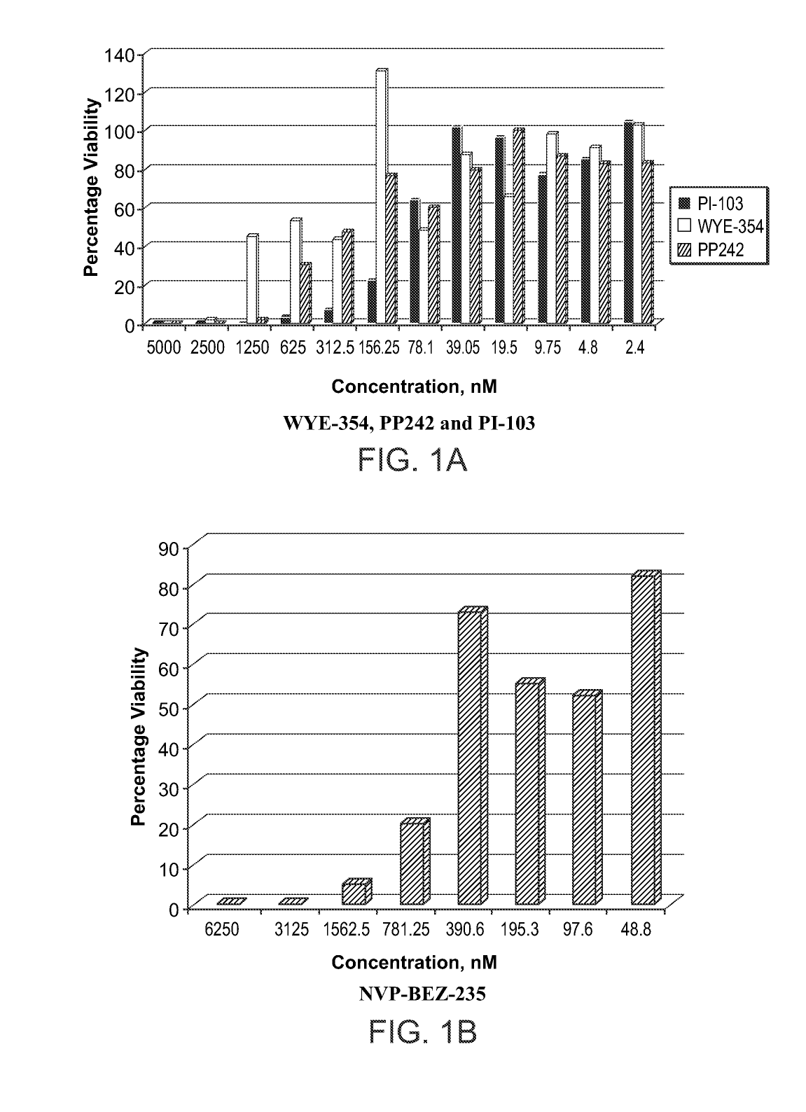

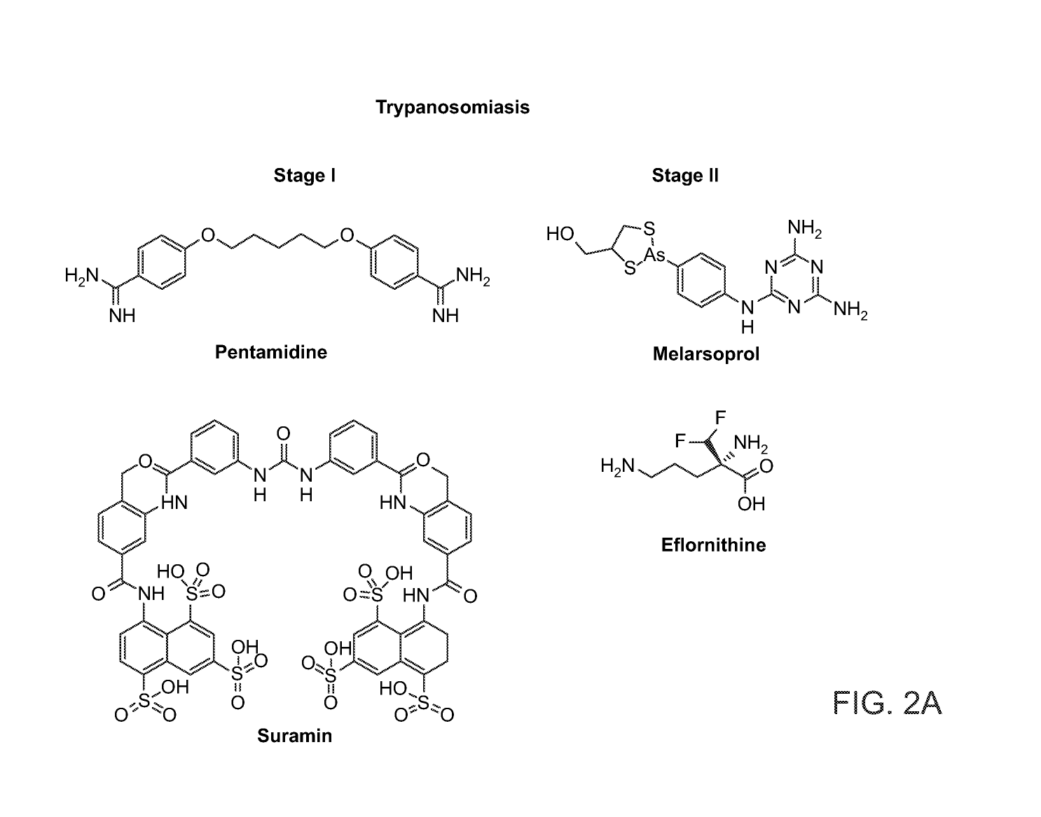

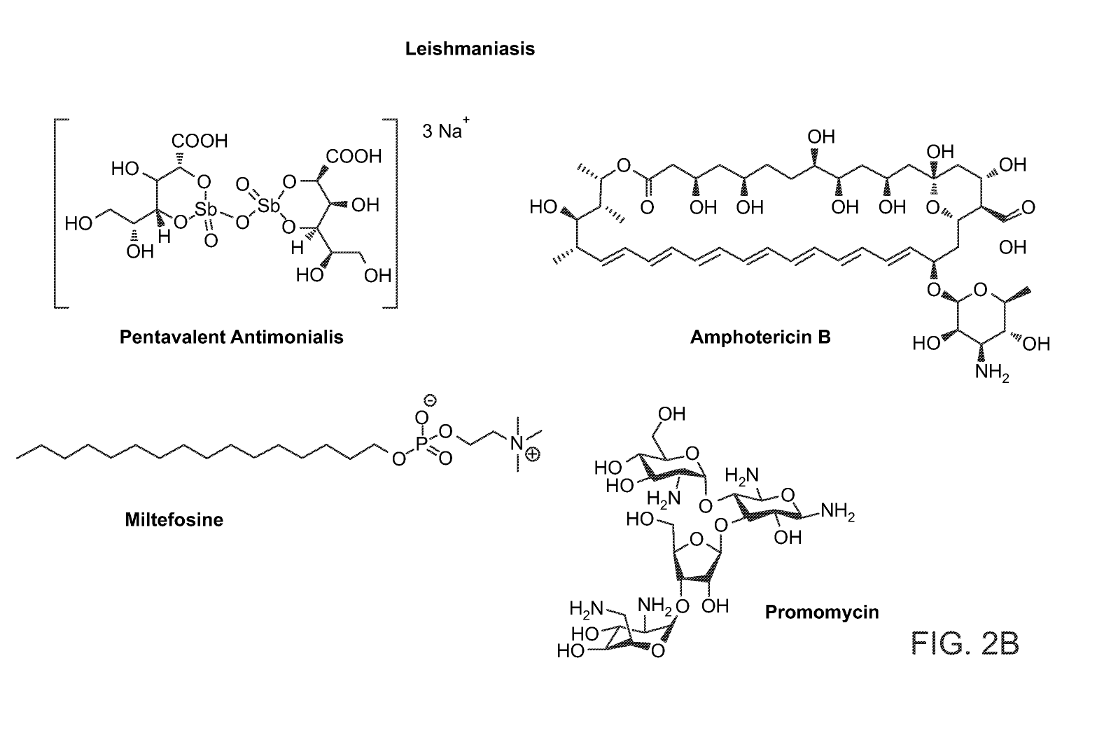

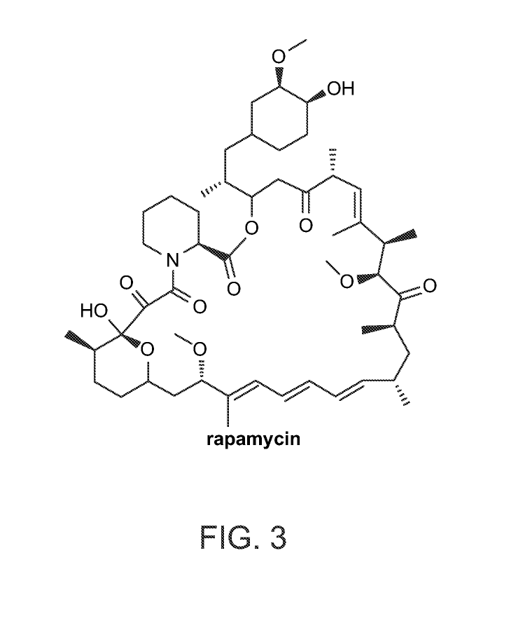

Presented herein are the selection and screening results of commercially available mTOR inhibitors against T. brucei and L. major as well as the design and synthesis of a variety of analogs of two non-limiting representative mTOR inhibitors, PI-103 and WYE-354. Eight mTOR kinase domain inhibitors were chosen for screening against whole parasites, including T. brucei brucei, T. brucei rhodesiense (human infective strain) and L. major promastigotes. NVP-BEZ-235, PI-103, WYE-354 and PP242 were all found to be potent anti-parasitic agents (FIGS. 1A and 1B). Three of the four were more potent with human infective T. brucei rhodesiense than with T. brucei brucei. PI-103 and WYE-354 were chosen for analog synthesis and structure activity relationship (SAR) analysis.

### Materials and Methods

Ethics Statement.

The animal experimental protocol (2010102/1) used for African trypanosome studies was reviewed and approved by the Ethical Committee IPBLN-CSIC of the Spanish Council of Scientific Research (CSIC). For T. cruzi, animal studies were approved by the Institutional Animal Care and Use Committee of New York University School of Medicine (protocol #81213), which is fully accredited by the Association For Assessment and Accreditation Of Laboratory Animal Care International (AAALAC). For L. major, animal studies were approved by the Animal Studies Committee at Washington University (protocol #20090086) in accordance with the Office of Laboratory Animal Welfare's guidelines and the Association for Assessment and Accreditation of Laboratory Animal Care International.

### Compounds

Inhibitor compounds were received from commercial vendors. PI-130, NVP-BEZ235, Ku-0063794, Pp242, and WYE-354 were obtained from Chemdea, Inc. (Ridgewood, N.J.). LY294002, LY303511, and Compound 401 were obtained from Tocris Biosciences (Ellisville, Mo.).

### In Vitro Experiments

Potency Assessment Against T. Brucei.

Assays were performed using the strain of T. brucei brucei Lister 427 adapted to the laboratory, and the human-infective strain T. b. rhodesiense (EATRO3 ETat1.2 TREU164 [37]). Both strains were grown and tested as bloodstream forms. To establish the EC₅₀, cultures of Trypanosoma brucei and T. b. rhodesiense were treated with two-fold increasing concentrations of compounds (with similar DMSO increasing concentration as control). Cell populations were measured at 72 hours with an Infinite F200 microplate reader (Tecan Austria GmbH, Austria); the determination of cell viability was carried out by the established colorimetric technique AlamarBlue® with modifications, a 96-well plate format spectrophotometric assay which measures the ability of living cells to reduce resazurin [38,39]. Data obtained with T. b. brucei Lister 427 were confirmed by manual counting in a Neubauer chamber for a direct microscopic examination to rule out multinucleated phenotypes that could mask the colorimetric assays, as well as the subtraction of solvent background to dismiss a potential solvent-derived fluorescence. Pentamidine was used as drug control for potency comparison, and T. b. brucei Lister 927 strain was included in our experiments to evaluate the adaptation to medium for the different strains as a variable condition.

Analysis of Morphological and Cell Cycle Alterations from Compound Treatment in T. brucei.

Flow cytometry was used to assess cell size and DNA content, to reveal a G1 arrest and inhibition of protein synthesis or G2 arrest and multinucleated cells. Briefly, bloodstream cells of T. brucei brucei Lister 427 strain in early log phase culture were treated with high dose (1 μM for PI-103, 2 μM for WYE-354 and Pp242 and 100 nM for NVP-BEZ235) of compounds for 16 hours, when the cells were pelleted and washed to remove all traces of drug. After permeabilization with 14, saponin (0.5 mg/mL final concentration), the culture was RNAse treated for 30 minutes (10 μg/mL final concentration) and stained with 20 μg/mL propidium iodide immediately before its acquisition in a FACscan cytometer. Cells incubated with equivalent concentration of drug solvent (DMSO) were included in each experiment as control population.

Potency Assessment Against T. Cruzi.

T. cruzi trypomastigotes from the Tulahuen strain stably expressing the β-galactosidase gene [40] were obtained from the supernatant of infected cultures of LLC-MK2 cells harvested between days 5 and 7. To remove amastigotes, trypomastigotes were allowed to swim out of the pellet of samples that had been centrifuged for 7 min at 2500 rpm.

Intracellular Replication.

5×10⁴ NIH/3T3 cells and 5×10⁴ trypomastigotes/per well were seeded in 96-well plates in DMEM supplemented with 2% FBS and Pen-Strep-Glut. DMEM did not contain phenol red to avoid interference with the assay absorbance readings at 590 nM. After 3 hours, compounds were added to a final volume of 200 μl/well at the indicated concentrations and mixed by pipetting. A 4 μM Amphotericin B solution (Sigma-Aldrich) was used as positive control. After 4 days of incubation at 37° C. 5% CO₂, 50 μl of PBS containing 0.5% of NP40 and 100 μM chlorophenol red-β-D-galactoside (CPRG) (Fluka) were added in each well. Plates were incubated at 37° C. for 4 hours and absorbance was read at 590 nm.

Extracellular Survival.

Free trypomastigotes were rinsed once and placed in 96-well plates at 100,000/well with the compounds in a final volume of 200 μl of DMEM without phenol red supplemented with 2% FBS, Pen-Strep-Glut and 100 μM CPRG. Plates were incubated for 24 h at 37° C. and absorbance was read at 590 nm.

Potency Assessment Against Leishmania.

Leishmania major strain FV1 (MHOM/IL/80/Friedlin) was grown in M199 media [41]. Leishmania donovani strain LdBob (MHOM/SD/62/1S-CL2D) were grown in modified M199 media as promastigotes (26° C.) [42]. Amastigote specific media (37° C.) was used for growth and differentiation of amastigotes [42]. L. donovani axenic amastigotes were passed once following differentiation prior to use. Cells were enumerated using a Coulter Counter (BD Biosciences); as amastigotes tend to grow in clumps, L. donovani axenic amastigotes were passed gently through a blunt 27-gauge needle prior to counting. For determination of EC₅₀s, log phase cells were inoculated at concentration of 10⁵/ml into appropriate media with compounds as indicated, and counted when the controls lacking drug had reached late logarithmic phase. The EC₅₀ is defined as the concentration of drug inhibiting 50% of control growth, and was calculated by linear regression analysis using SigmaPlot 2000.

Cell Size and DNA Content Analysis in Leishmania.

L. major log phase promastigotes were inoculated at a concentration of 10⁶ cells/ml into media with compounds as indicated, and incubated overnight with varying drug concentrations to assess cell size and DNA content. For cell size, forward scatter of live promastigotes was measured by a FACS flow cytometer (Becton Dickinson), utilizing dye exclusion with 5 μg/ml propidium iodide (PI) to gate for live cells. DNA content was determined by flow cytometry using fixed and permeabilized L. major stained with PI as previously described [43,44], but reducing the incubation time with PI and RNase A from 1 hour to 30 minutes. Histogram analysis was performed using CellQuest 3.1 software (BD Bioscience).

### In Vivo Experiments.

Drug Dosage.

The targeted dosage of inhibitors was determined based on the pharmacokinetic studies disclosed by Maira, et al. [45]. The goal was to test NVP-BEZ235 in the animal models at the highest dose achievable without inducing toxicity. For L. major, 12.5 mg/kg orally was the highest tolerable dose while 30 mg/kg intraperitoneally was used for the T. cruzi infections. A lower dose was initially used in T. brucei, 5 or 10 mg/kg intraperitoneally.

T. brucei:

Balb/c female mice were obtained from Jackson Laboratories (Bar Harbor, Me.). All animal protocols in M.N. lab were approved by the CSIC-IPBLN Committee on Use and Care of Laboratory Animals. Balb/C mice were infected with 10⁴ cells of an early log phase culture of T. b. rhodesiense EATRO3; 72 hours after infection the mice were arbitrarily separated into three independent groups, daily treated with 5 or 10 mg/kg NVP-BEZ235, or DMSO, via intraperitoneal injection for four days. The parasitemia was checked at days 3, 5, 7, 11 and 14 post-infection in alive mice: in those cases the parasitemia was too low to detect by Neubauer chamber count, the extracted blood was incubated in a 24-well plate with HMI-9 medium supplemented with 20% SBFi at 37° C. with 5% CO₂, and positive wells were confirmed by direct visualization of parasites. Humanitarian sacrifice was executed, according to Ethic Commission of Animal Welfare directions, and necropsies were done in order to identify any physical side effect related to administration.

T. cruzi:

Balb/c mice were inoculated intraperitoneally with 10⁵ trypomastigotes from T. cruzi Y strain expressing firefly luciferase (kindly provided by Dr. Barbara Burleigh, Harvard University). On day 7 post-infection, mice were anesthetized with ketamine/xylazine and injected with 3 mg of D-Luciferin Potassium Salt (Gold Biotechnology) at 20 mg/ml in PBS and imaged in the IVIS Lumina II (Caliper Life Sciences). On day 8, groups of five mice were injected ip with either 30 mg/kg of NVP-BEZ235 in DMSO or only DMSO, as control. Mice were treated for 5 days and imaged again on day 13. Data is expressed as the ratio between luciferase units in day 13 versus day 7 to determine the progression of infection with and without drug treatment.

L. major:

Mouse experiments were done in compliance with policies approved by the Animal Review Board at Washington University. Mice were infected with luciferase expressing L. major (LmFV1LucTK-1) and analyzed by bioluminescent imaging as described [46]. Balb/c mice were infected with 10⁵ L. major metacyclic stage parasites purified by gradient centrifugation [47]. Luminescence was measured using an IVIS 100 instrument and analyzed with Living Image software version 2.60. NVP-BEZ235 was resuspended in DMSO and applied at 12.5 mg/kg/day by oral gavage for 10 days, with treatment starting day 17 post-infection. At this dose the mice showed significant weight loss, suggesting that this dosage was the highest practicable, as dosing intraperitoneally at 25 mg/kg/day was lethal.

### Results

### Compound Selection.

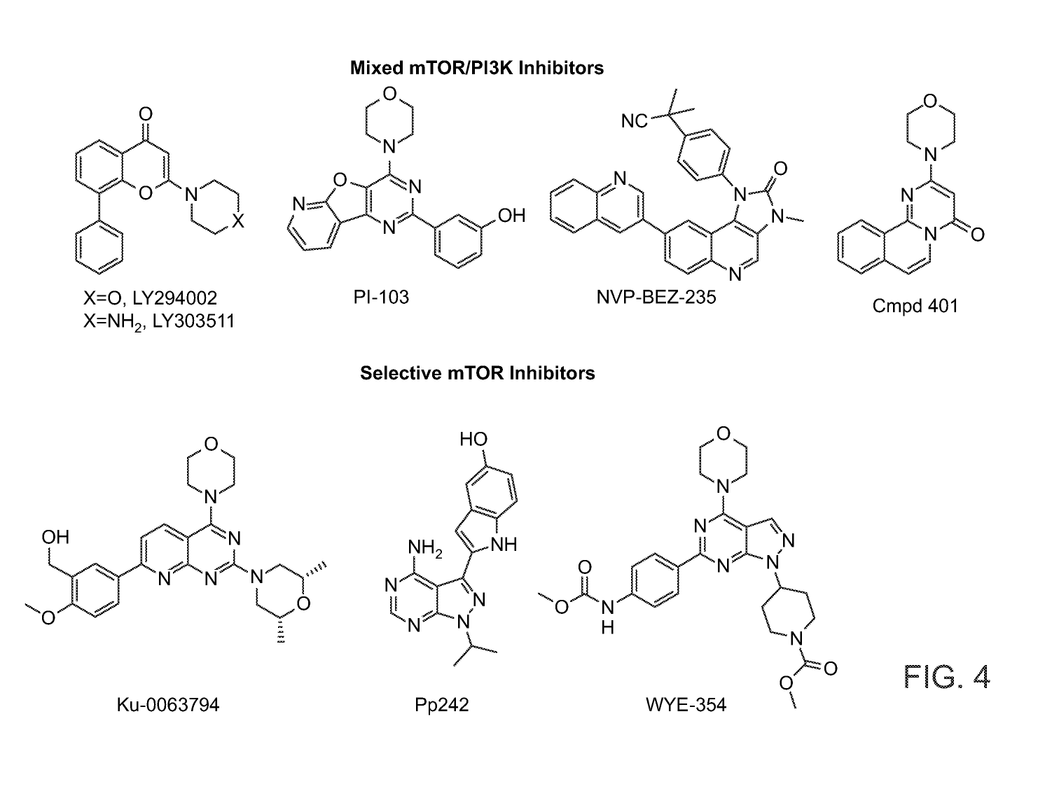

Eight commercially-available compounds (FIG. 4, Table 1) were selected to profile for activity against Trypanosoma brucei, T. cruzi and two species of Leishmania, cutaneous L. major and visceral L. donovani. FIG. 1 shows EC₅₀ determinations of WYE-354, PP242, PI-103, and NVP-BEZ-235 in T. brucei rhodesiense. To identify inhibitors of trypanosome TORs or PI3Ks, a range of compounds with varied potencies and selectivities against mTOR/PI3K were selected. In mammalian cells, compounds Ku-0063794 [21,22], Pp242 [18], and WYE-354 [48] inhibit the kinase domain of mTOR selectively with low nanomolar IC₅₀ values. LY294002 is a mixed inhibitor targeting both mTOR/PI3K [49], and many analogs have been made (including LY303511, which inhibits mTOR-dependent and independent pathways, but does not inhibit PI3Ks [50,51]). PI-103 inhibits PI3Ks with high potency and mTOR with a reported 20 nM IC₅₀ [52,53,54]. Compound 401, a compound structurally related to LY303511, inhibits mTOR and cellular growth at low micromolar concentrations [55], while NVP-BEZ235 inhibits both PI3Ks and mTOR with sub-nanomolar IC₅₀ values [56,57].

TABLE 1
Selectivity profile of the selected inhibitors against human enzymes.

| | Inhibition | | | | | | |
|---|---|---|---|---|---|---|---|
| | of Cell | | PI3K | | | | |
| | Growth | mTOR | p110α | p110β | p110δ | p110γ | |
| Compound | EC₅₀ (μM) | IC₅₀ (μM) | IC₅₀ (μM) | IC₅₀ (μM) | IC₅₀ (μM) | IC₅₀ (μM) | Refs. |
| NVP-BEZ235 | 0.05 | <0.01 | 0.004 | 0.075 | 0.007 | 0.005 | [24, 45, 57] |
| PI-103 | 0.5 | 0.02 | 0.0036 | 0.003 | | 0.25 | [53, 66] |
| LY294002 | | 5 | 0.73 | 0.31 | 1.06 | 6.6 | [67, 68] |
| LY303511 | 1 | | | | | | [50] |
| Compound 401 | 1 | 5.3 | >100 | | | | [55, 69] |
| Pp242 | 0.04 | 0.008 | 2.8 | 2.2 | 0.1 | 1.3 | [18, 19] |
| WYE-354 | 0.03 | 0.004 | 1 | | | | [20] |
| Ku-0063794 | 0.03 | 0.01 | 8.9 | >30 | >5 | >30 | [21, 22] |

Selective mTOR Inhibitors 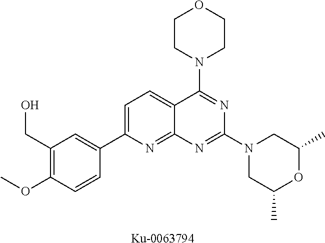 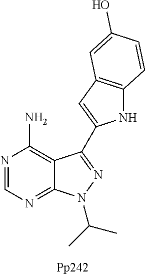 [SMILES: CC(C)n1nc(-c2cc3cc(O)ccc3[nH]2)c2c(N)ncnc21] 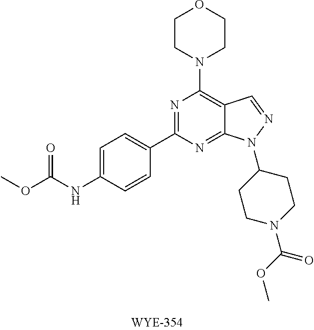 [SMILES: COC(=O)Nc1ccc(-c2nc(N3CCOCC3)c3cnn(C4CCN(C(=O)OC)CC4)c3n2)cc1]
Mixed mTOR/P13K Inhibitors

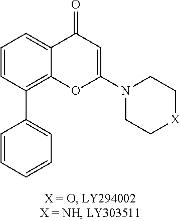

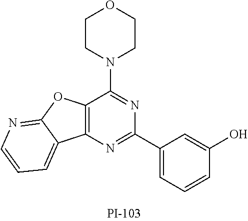 [SMILES: Oc1cccc(-c2nc(N3CCOCC3)c3oc4ncccc4c3n2)c1]

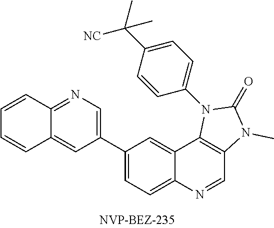 [SMILES: Cn1c(=O)n(-c2ccc(C(C)(C)C#N)cc2)c2c3cc(-c4cnc5ccccc5c4)ccc3ncc21]

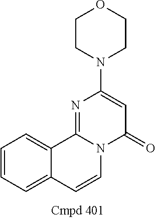 [SMILES: O=c1cc(N2CCOCC2)nc2c3ccccc3ccn12]

The table below presents the effects of the compounds on human and trypanosome cells.

### In Vitro Testing of Inhibitors.

The compounds were first tested against parasites grown in vitro. For T. brucei and Leishmania donovani, it is possible to cultivate free parasites in vitro as the infective stage forms: bloodstream form (BSF) for T. brucei, and axenic amastigotes for L. donovani. compounds were also tested against L. major promastigotes (the stage carried normally by the insect vector). To study infective forms of T. cruzi, compounds were added simultaneously with trypomastigotes to 3T3 fibroblast host cells and incubated for four days. This protocol thus monitors all steps of the T. cruzi infective cycle (entry, differentiation and replication as amastigotes) as well as potential effects mediated through host cell PI3Ks. The results of the in vitro assessment of this inhibitor collection are shown in Table 2.

TABLE 2
Summary of potency data of mTOR/PI3K inhibitors against trypanosomatid cultures.
Effective concentration (EC₅₀) values are shown in micromolar concentrations except as noted.

| | Leishmania sp. | | | Trypanosoma sp. | | |
|---|---|---|---|---|---|---|
| Compound | L. major^{a} | L. donovani^{a} | L. donovani^{b} | T. cruzi^{c} | T. b. brucei^{d} | T. b. rhodesiense^{d} |
| PI-103 | 0.32 ± 0.16* | 1.05 ± 0.28 | 0.62 ± 0.41 | >25 | 0.214 ± 0.036 | 0.105 ± 0.01 |
| NVP-BEZ235 | 0.11 ± 0.05 | 0.14 ± 0.08 | 0.07 ± 0.04 | 0.12 | <10 nM | 0.73 ± 0.06 nM |
| LY294002 | >25 | — | >5 | >50 | >2 | — |
| LY303511 | >25 | — | >5 | >10 | >2 | — |
| Compd 401 | >25 | — | >5 | >10 | >10 | — |
| Pp242 | 2.4 ± 0.8# | 0.42 ± 0.03 | 0.50 ± 0.09 | >10 | 0.48 ± 0.1 | 0.166 ± 0.015 |
| WYE-354 | 4.1 ± 0.3* | 5.95 ± 0.84 | 6.10 ± 1.73 | >10 | 0.58 ± 0.26 | 0.78 ± 0.08 |
| Ku-63794 | >25 | — | >5 | >10 | 0.89 ± 0.14 | — |

^{a}promastigotes, average of three replicates;
^{b}axenic amastigotes, average of three replicates;
^{c}trypomastigotes, average of three replicates, within ±10.2%;
^{d}bloodstream form, average of three replicates.
*p < 0.05 for L. major vs. L. donovani promastigotes;
#p < 0.05 for L. major promastigotes vs. L. donovani promastigotes or amastigotes.

Table 2A shows the structures of PI-103 and WYE-354 analogs that were synthesized and tested. Table 2B shows the biological assessment of PI-103 and WYE-354 analogs versus trypanosomatid parasites.

TABLE 2A

| Structure | Compd Number | Class |
|---|---|---|
| 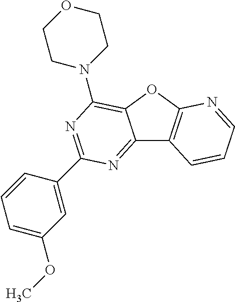 [SMILES: COc1cccc(-c2nc(N3CCOCC3)c3oc4ncccc4c3n2)c1] | NEU-0000288 | PI-103 analog |
| 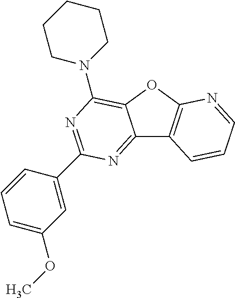 [SMILES: COc1cccc(-c2nc(N3CCCCC3)c3oc4ncccc4c3n2)c1] | NEU-0000289 | PI-103 analog |
| 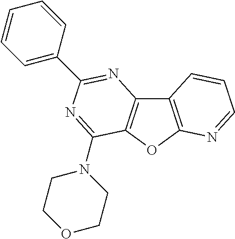 [SMILES: c1ccc(-c2nc(N3CCOCC3)c3oc4ncccc4c3n2)cc1] | NEU-0000290 | PI-103 analog |
| 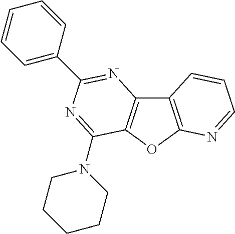 [SMILES: c1ccc(-c2nc(N3CCCCC3)c3oc4ncccc4c3n2)cc1] | NEU-0000291 | PI-103 analog |
| 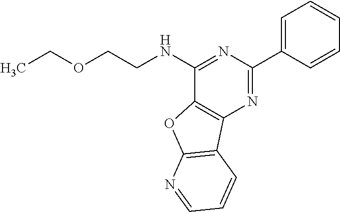 [SMILES: CCOCCNc1nc(-c2ccccc2)nc2c1oc1ncccc12] | NEU-0000292 | PI-103 analog |
| 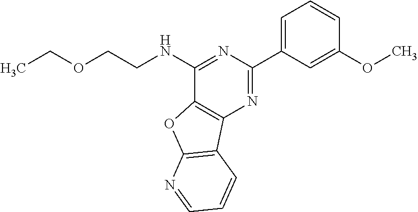 [SMILES: CCOCCNc1nc(-c2cccc(OC)c2)nc2c1oc1ncccc12] | NEU-0000361 | PI-103 analog |
| 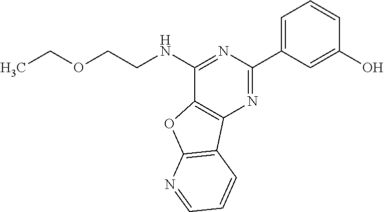 [SMILES: CCOCCNc1nc(-c2cccc(O)c2)nc2c1oc1ncccc12] | NEU-0000362 | PI-103 analog |
| 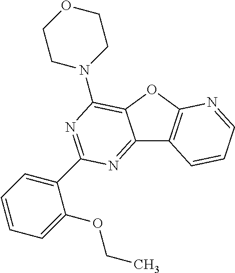 [SMILES: CCOc1ccccc1-c1nc(N2CCOCC2)c2oc3ncccc3c2n1] | NEU-0000363 | PI-103 analog |
| 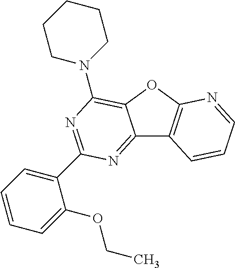 [SMILES: CCOc1ccccc1-c1nc(N2CCCCC2)c2oc3ncccc3c2n1] | NEU-0000364 | PI-103 analog |
| 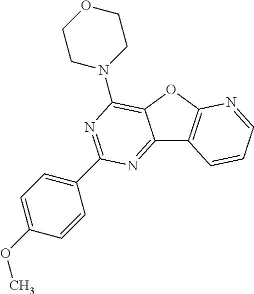 [SMILES: COc1ccc(-c2nc(N3CCOCC3)c3oc4ncccc4c3n2)cc1] | NEU-0000365 | PI-103 analog |
| 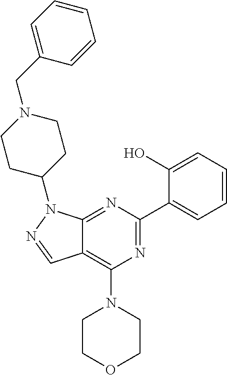 [SMILES: Oc1ccccc1-c1nc(N2CCOCC2)c2cnn(C3CCN(Cc4ccccc4)CC3)c2n1] | NEU-0000231 | WYE analog |
| 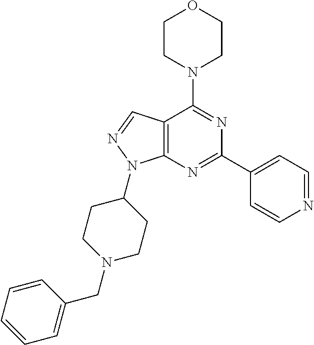 [SMILES: c1ccc(CN2CCC(n3ncc4c(N5CCOCC5)nc(-c5ccncc5)nc43)CC2)cc1] | NEU-0000232 | WYE analog |
| 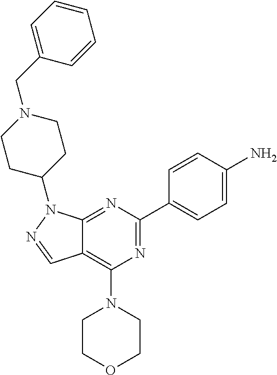 [SMILES: Nc1ccc(-c2nc(N3CCOCC3)c3cnn(C4CCN(Cc5ccccc5)CC4)c3n2)cc1] | NEU-0000233 | WYE analog |
| 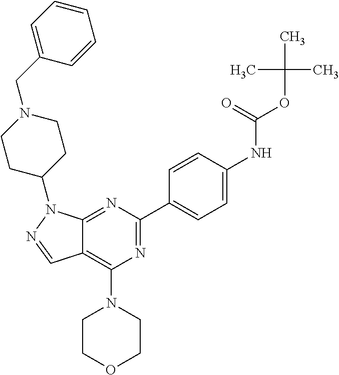 [SMILES: CC(C)(C)OC(=O)Nc1ccc(-c2nc(N3CCOCC3)c3cnn(C4CCN(Cc5ccccc5)CC4)c3n2)cc1] | NEU-0000234 | WYE analog |

TABLE 2B

| | | | | | | T. brucei |
|---|---|---|---|---|---|---|
| | | | | | L. donovani | rhod |
| | | T. Cruzi (μM) | L. major | L. donovani | Axenic | IC50, |
| | | IC50, growth | Promastigote | Promastigote | Amastigote | growth |
| Compd | | inhibition | IC50, growth | IC50, growth | IC50, growth | inhibition |
| Number | Class | (μM) | inhibition (μM) | inhibition (μM) | inhibition (μM) | (μM) |
| NEU- | PI-103 | 3.7 | 4.07 | | ~20 | |
| 0000288 | analog | | | | | |
| NEU- | PI-103 | 3.9 | 7.65 | | ~20 | |
| 0000289 | analog | | | | | |
| NEU- | PI-103 | 1.5 | >20 | | >20 | |
| 0000290 | analog | | | | | |
| NEU- | PI-103 | 12.0 | >10 | | >10 | |
| 0000291 | analog | | | | | |
| NEU- | PI-103 | >50 | 4 | | 12.5 | |
| 0000292 | analog | | | | | |
| NEU- | PI-103 | >50 | | | | >5 |
| 0000361 | analog | | | | | |
| NEU- | PI-103 | >50 | | | | >1 |
| 0000362 | analog | | | | | |
| NEU- | PI-103 | >50 | | | | >5 |
| 0000363 | analog | | | | | |
| NEU- | PI-103 | >50 | | | | >5 |
| 0000364 | analog | | | | | |
| NEU- | PI-103 | >50 | | | | >5 |
| 0000365 | analog | | | | | |
| NEU- | WYE | | 2.78 | 8.01 | 3.02 | 2.23 |
| 00000231 | analog | | | | | |
| NEU- | WYE | | 6.54 | 8.06 | 2.43 | 3.05 |
| 00000232 | analog | | | | | |
| NEU- | WYE | | <25.00 | ~25.00 | NT | 3.7 |
| 00000233 | analog | | | | | |
| NEU- | WYE | | 5.49 | 6.34 | 6.9 | 2.19 |
| 00000234 | analog | | | | | |

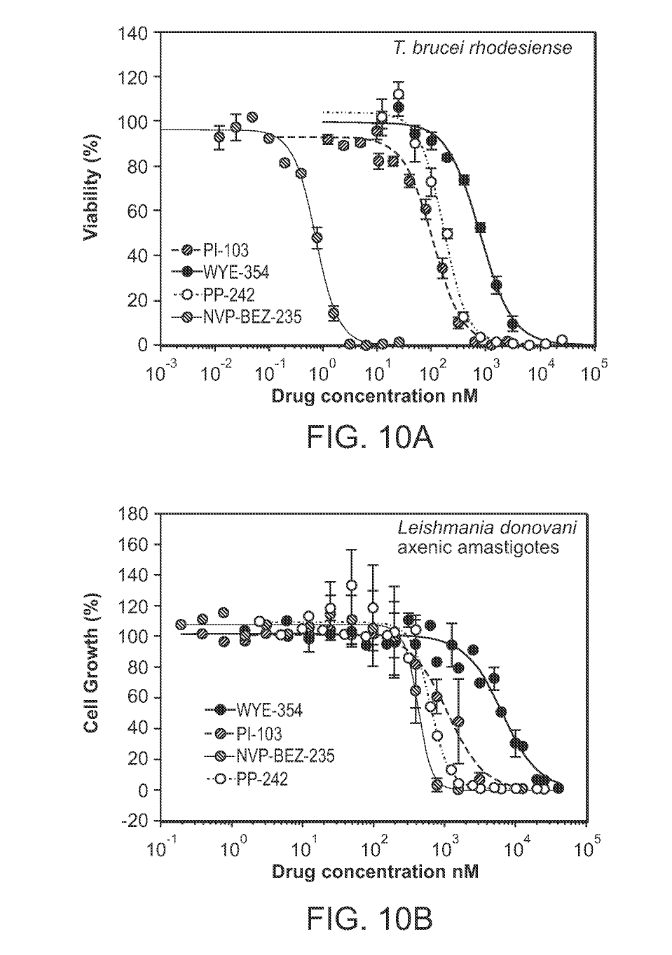

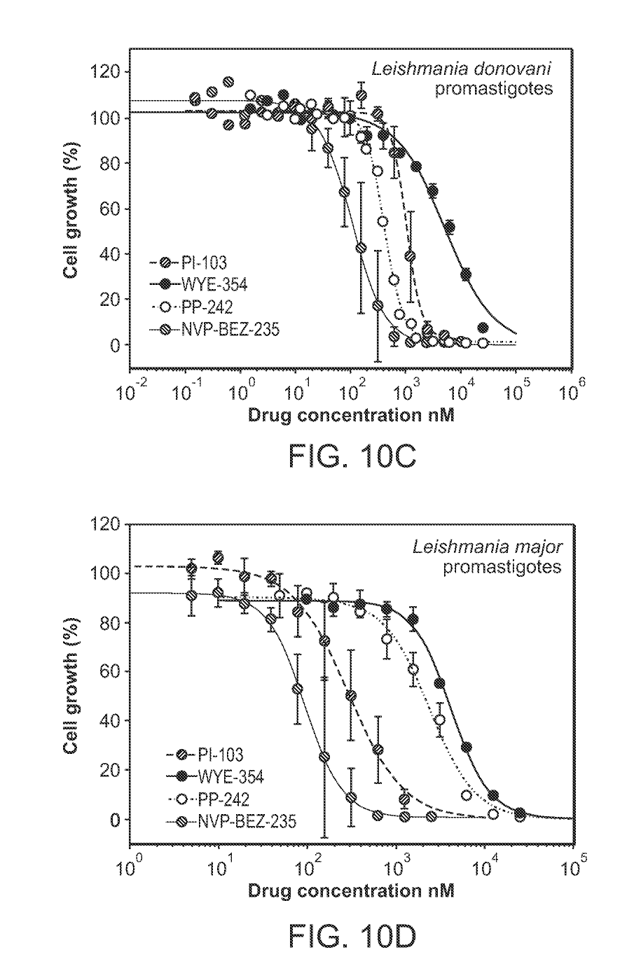

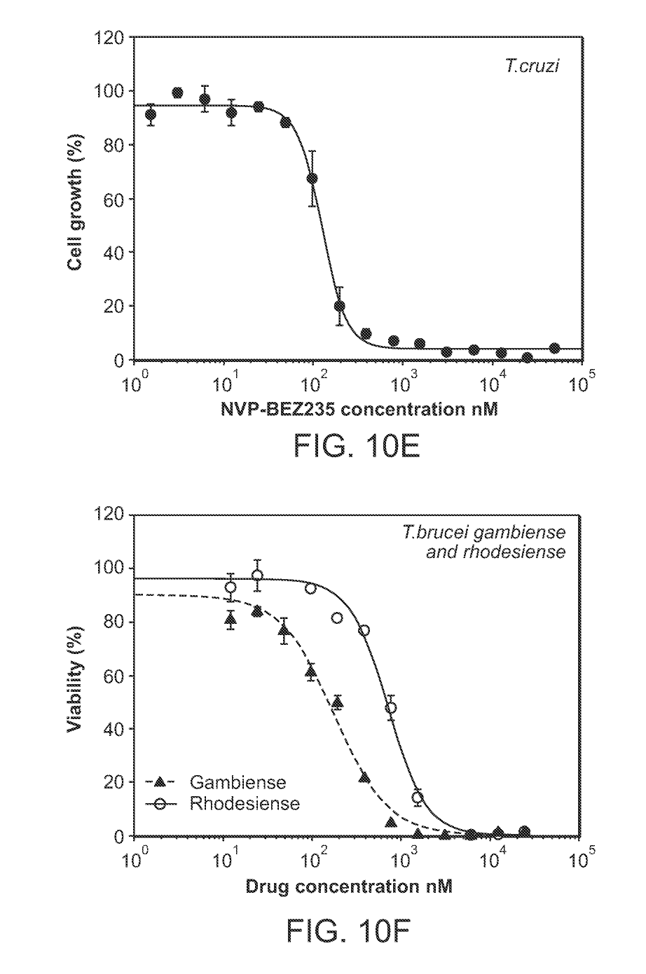

The most potent compound against all the species tested was NVP-BEZ235, showing nanomolar potency against BSF T. brucei brucei (Lister 427) and sub-nanomolar activity (730 pM) against the human-infective EATRO3 strain of T. b. rhodesiense (FIG. 10A, F). Interestingly, the BSF T. brucei gambiense, ELIANE strain [37] was even more sensitive, with an EC₅₀ of 179 pM. PI-103 showed good activity against T. b. brucei and T. b. rhodesiense cultures (200 and 100 nM, respectively). The other inhibitors showed micromolar activity against T. brucei brucei, and, as observed with NVP-BEZ235, these inhibitors are approximately ten-fold more potent against T. b. rhodesiense. The variation in potency of NVP-BEZ235 across different strains of T. brucei, (including the T. brucei brucei Lister 927 strain) is comparable to that seen in similar studies of pentamidine, an established drug (Table 3).

TABLE 3
Summary of potency data of NVP-BEZ235 against T. brucei
brucei, T. b. rhodesiense, and T. b. gambiense,
compared to the known drug pentamidine

EC₅₀ (nM)

| | NVP-BEZ235 | Pentamidine |
|---|---|---|
| T. b. brucei 427 | 16.3 ± 4.7 | 4.2 ± 0.2 |
| T. b. brucei 927 | 1.7 ± 0.5 | 30.3 ± 10.4 |
| T. b. rhodesiense EATRO3 | 0.73 ± 0.06 | 3.7 ± 0.4 |
| T. b. gambiense ELIANE | 0.18 ± 0.2 | 2.5 ± 0.5 |

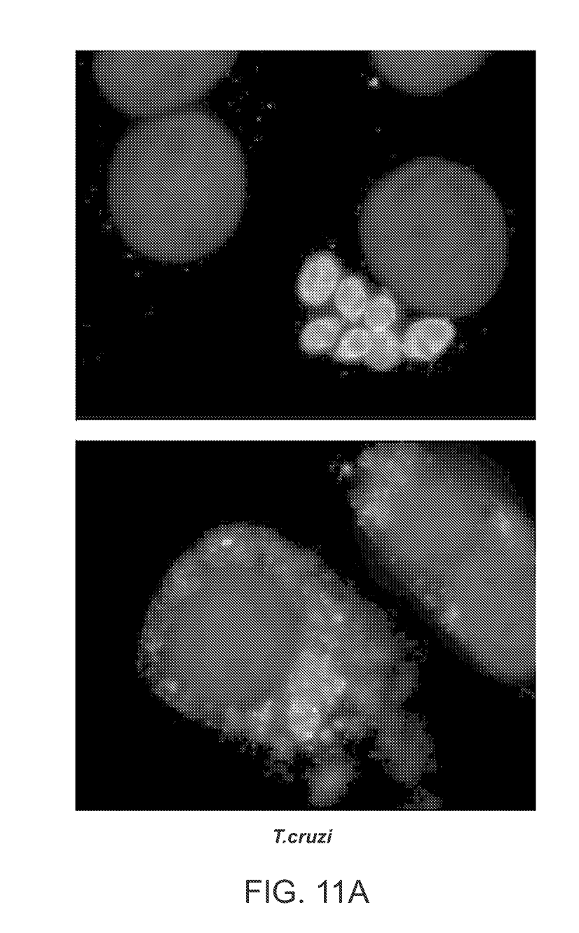

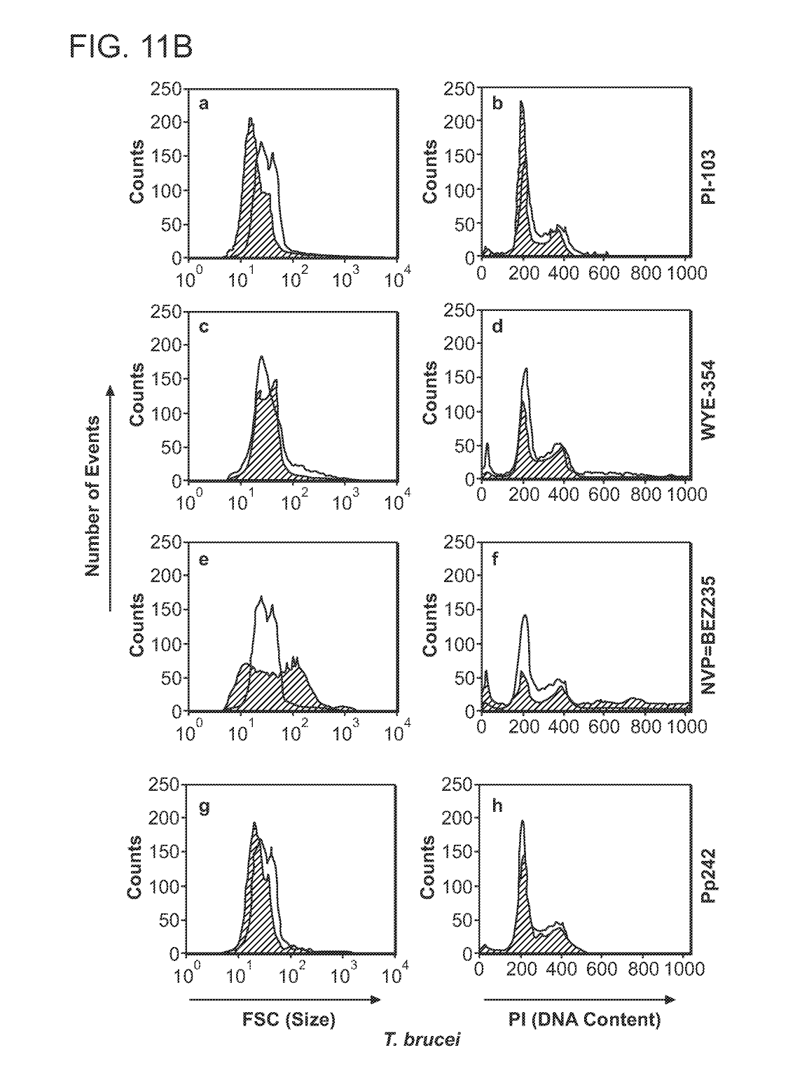

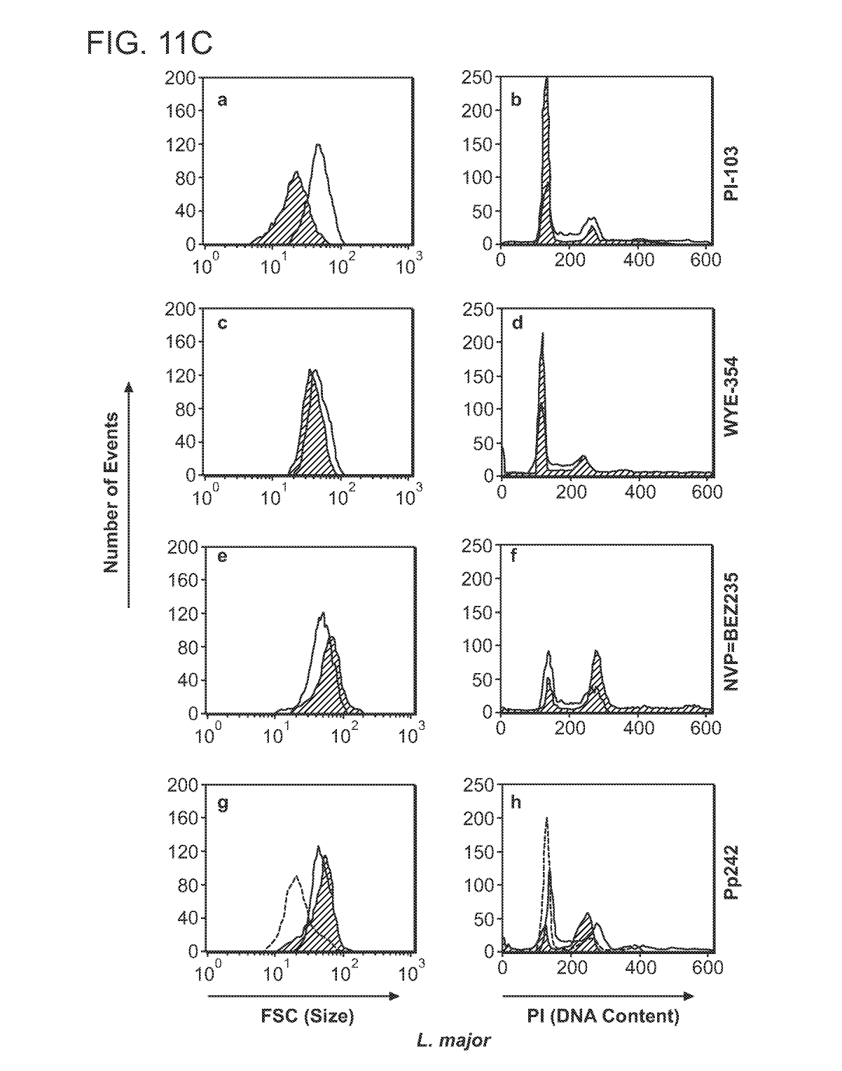

In infections of host cell fibroblasts by infective trypomastigotes, T. cruzi was refractory to all the inhibitors tested, except for NVP-BEZ235 (EC₅₀=120 nM, FIG. 10E). For this compound amastigotes lysis within host cells was observed after three days when the drug was dosed at 350 nM (˜3× the EC₅₀; FIG. 11A). In FIG. 11A, NIH-3T3 host cells were incubated with T. cruzi trypomastigotes for 2 hours before addition of NVP-BEZ235 (350 nM). Cells were incubated for 4 days during which time the parasites differentiate and replicate as amastigotes. At that time cells were fixed and stained with an anti-T. cruzi antiserum (green) and DAPI to visualize DNA (blue). The upper panel shows control cells with intact amastigotes, and the lower panel shows debris of parasite proteins throughout the host cell cytoplasm. In contrast, NVP-BEZ235 showed little activity (EC₅₀>50 μM) against free trypomastigotes, which do not replicate outside of host cells. This suggests that NVP-BEZ235 could act specifically against the amastigotes stage, or by activation of host cell responses.

For Leishmania, NVP-BEZ235 and PI-103 showed submicromolar inhibition across both species and stages (70-140 or 320-1050 nM respectively), while Pp242 and WYE-354 showed modest activity (0.4-2.4 μM or 4-6 μM respectively, FIG. 10B-D). The remaining four inhibitors (LY294002, LY303511, Compound 401 and Ku-63794) were inactive against L. major promastigotes and L. donovani axenic amastigotes at the highest concentration tested and were not tested against L. donovani promastigotes. While some compounds showed statistically significant differences amongst the Leishmania strains/species, the differences were modest and not studied further.

### Phenotypic Effects Elicited by PI3K Inhibitor Treatments

We examined effects of several of the strongest inhibitors on cell size, shape and/or DNA content, since mTOR and PI3K inhibitors affect the size of both mammalian and T. brucei cells and induce characteristic growth phase arrests [29,58].

Sixteen hour treatment of BSF T. brucei brucei with drug at an effective concentration (described below) produced two different types of effects on cellular DNA content (FIG. 11B). FIG. 11B and FIG. 11C show the fluorescence-activated cell sorter (FACS) analysis of cell size (Forward Scatter, FSC) and DNA content after drug treatment. In FIG. 11B, bloodstream form culture of T. b. brucei was subjected to different drugs, indicated to the side, and analyzed by FACS for cell size and DNA content stained by propidium iodide. Cell cultures were incubated during 16 h with PI-103 (1 μM), WYE-354 (2 μM), Pp242 (2 μM) and NVP-BEZ235 (100 nM), represented with dark lines, and with DMSO as control population, represented as shaded area. Two drugs, (PI-103 and Pp242), tested at 1 μM and 2 μM, respectively) induced G1 arrest, an effect maintained even at low concentrations of Pp242 (200 nM, data not shown). While the inhibitor PI-103 showed a clearly defined profile in cell cycle progression, NVP-BEZ235 produced a combination of effects on the cell cycle progression at 0.1 μM, including the appearance of zoids (anucleated cells) [59] and multinucleated cells. This relatively high dose of NVP-BEZ235 (10× the EC₅₀) produced a reduction of G1 and G2 cells. Finally, treatment of T. b. brucei cells with WYE-354 resulted in no significant variations in cell cycle, with a small but noticeable reduction in cell size.

We examined the effect on cell size and DNA content for the four compounds that displayed activity against L. major promastigotes, using drug concentrations (EC₆₀-EC₉₀) that were strongly inhibitory, but without inducing complete growth arrest or cellular toxicity (as evidenced by PI exclusion tests, not shown). For all concentrations tested, both PI-103 and WYE-354 treatment induced a G1 arrest and a decrease in cell size (FIG. 11C) as seen for start here mTORC1 inhibitors in mammalian cells. FIG. 11C shows results from the treatment of Leishmania major promastigotes. Dark lines are WT parasites examined when in logarithmic growth phase; shaded areas are parasites grown in the presence of the indicated concentration of drug. Cell size (FSC) and DNA content (PI staining) were determined as indicated in Panel B and/or as described in the Methods. The subpanels show data for PI-103 (4 μM, ˜EC90); WYE-354 (25 μM, ˜EC60); NVP-BEZ235 (0.5 μM, EC90) and Pp242 (dashed lines 12.5 μM/˜EC90, shaded area 25 μM/˜EC90). In contrast, NVP-BEZ235 treatment induced G2 growth arrest and increased promastigote size in a manner similar to the cell phenotype observed in mammalian cells exposed to mTORC2 inhibitors. Microscopy data suggests that the G2 arrest was actually due to altered cytokinesis, as evidenced by the abundance of individual cells that contain 2 nuclei and kinetoplasts (data not shown), again consistent with known effects of mTORC2 inhibition in mammalian cells. Though PI-103, WYE-354 and NVP-BEZ235 generated single phenotypes, Pp242 generated two different phenotypes depending on the drug concentration. At lower concentrations, Pp242 induced a decrease in cell size and a G1 arrest, while at higher concentrations a G2 arrest and increase in cell size was observed (FIG. 11C). This suggests the likelihood of inhibition of multiple targets with various affinities within the parasite.

### In Vivo Tests

The most active inhibitor, NVP-BEZ235, was chosen for testing in appropriate animal models of T. brucei rhodesiense, T. cruzi, and L. major infection. Using the highest tolerable doses appropriate for each infection model, no efficacy was observed against either T. cruzi (30 mg/kg, 5 days, intraperitoneal) or L. major (12.5 mg/kg/day, 10 days, oral gavage) (data not shown). Weight loss was observed in drug-treated mice infected with L. major and higher drug doses were lethal.

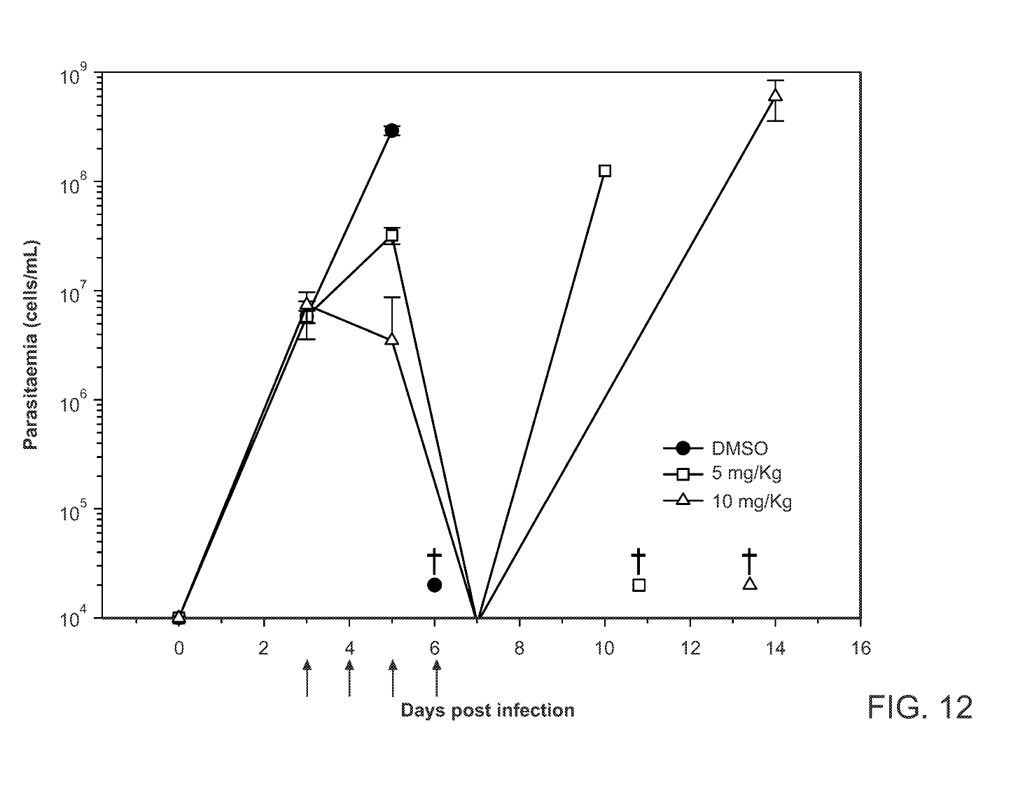

In contrast, a marked decrease in parasitemia was observed by intraperitoneal dosage (5 or 10 mg/kg) of NVP-BEZ235 in T. brucei rhodesiense infected mice. Drug was administered once per day, for four days. A dramatic decrease in parasitemia was observed within two days, below the detection limit of 10⁴ parasites/mL. All mice in the untreated group died the 6th day post-inoculation, while the mean survival day (MSD) for animals treated with 5 mg/kg of NVP-BEZ235 was extended to 10.8 (±2.4) days. The MSD of mice treated with 10 mg/kg increased to 13.4 (±3.3) days, doubling the survival of the control group (FIG. 12). FIG. 12 shows the trypanocidal activity of NVP-BEZ235 in an acute mouse infection model. The arrow indicates the drug dosing schedule. The mean parasitemia for each group is represented for each day up to the death of all mice in a group. The mean survival day (MSD) is labeled in the graphic with daggers.

These experiments have identified small molecules useful for further medicinal chemistry optimization and for gaining a better understanding of molecular pharmacology. Noting the functional and structural homology between TOR and PI3Ks in humans and trypanosomatids and encouraged by the surprising growth inhibitory phenotype resulting from suppression of TOR in T. brucei [60] and L. major [30], we identified and procured eight established inhibitors for assessment against parasite cultures. Several of these inhibitors inhibited parasite growth in all species/strains tested, and one, NVP-BEZ235, reduced parasitemia in an animal model of T. brucei rhodesiense infection, significantly extending survival.NVP-BEZ235 showed very potent inhibition of T. brucei growth, with a phenotype similar to that seen previously in genetic studies of TOR and TbVps34 [28]. A modest treatment regime (10 mg/kg, for four days) was able to eliminate 80% of the parasites in T. b. rhodesiense infections. The in vitro potency (179 pM EC₅₀) observed against T. b. gambiense was exceedingly high, at a level rarely seen against these protozoan parasites. The reason for the difference in potency between T. b. rhodesiense and gambiense is not known at this time. While not limited by any mechanism of action, T. b. gambiense, with a lower generation rate and poorer adaptation to culture than T. b. rhodesiense, may be more affected by an antiproliferative drug as NVP-BEZ235. This difference was also observed between T. b. brucei Lister 427 and 927 (Table 3). The potency of this compound and the complex cell cycle phenotype observed suggest that the compound likely has a number of molecular targets in T. brucei, perhaps affecting other essential cellular functions besides cell proliferation.

T. cruzi was relatively insensitive to the inhibitors compared to T. brucei. While not limited by any mechanism of action, this may arise from the fact that T. cruzi trypomastigotes, the form of the parasite that proliferates in the human, only replicates in the intracellular environment. As a consequence, compounds need to cross the plasma membrane of the host cell to have access to T. cruzi, while T. brucei is directly accessible to the drugs in the bloodstream. When NVP-BEZ235 was tested against free, non-replicating T. cruzi trypomastigotes, it was inactive, while it induced lysis of intracellular T. cruzi amastigotes. This raises the possibility that this compound acts either specifically against the amastigote stage, or through effects on host cell PI3Ks, or some combination. The involvement of host cell PI3K and mTOR pathways in immune evasion has been recently reported for L. donovani [61].

Leishmania showed a range of sensitivities to the panel of inhibitors, with the most potent compounds active at sub-micromolar concentrations. This may be compared to the efficacy of current front line anti-leishmanial agents, whose potencies when measured by methods similar to those described here range from 30 nM for amphotericin B to 15 uM for antimonial based compounds (Seifert et al, 2011).

The EC₅₀ values for the compounds tested were similar for L. major promastigotes, L. donovani promastigotes and L. donovani axenic amastigotes, suggesting that preliminary screening against a single form could be sufficient in the future. However, despite its potency against L. donovani axenic amastigotes, high doses of NVP-BEZ235 against L. major infections of mice showed no therapeutic effect (data not shown). As discussed above for T. cruzi, the lack of efficacy in the animal model of L. major could reflect a similar need for the drug to traffic to the phagolysosomal compartment where Leishmania reside. The 12.5 mg/kg regime tested here resulted in severe weight loss (not shown) suggesting attempts to treat with higher doses of NVP-BEZ235 would result in significant toxicity.

Current data suggest there are at least 12 members of the PI3 Kinase protein superfamily, for which the phenotypic effects of inhibition or genetic deletion in T. brucei or Leishmania are known for only five. Thus, it is unknown what the likely cellular target may be. However, with an eye towards initial identification of specific targets potentially involved in the activity of NVP-BEZ235, we note a similar cell cycle phenotype that Hall et al. reported upon RNAi knockdown of TbVsp34 [28], including the appearance of zoids, multinucleated cells and reduction of G1 and G2 (FIG. 11B). Barquilla et al. also showed the same pattern after RNAi of TOR2 [35].

In trypanosomes and mammals, TORC1 inhibition is known to result in G1 arrest and debreased cell size, while TORC2 results in G2 arrest and increased cell size [29,33,62,63,64,65]. In both T. brucei and Leishmania PI-103 resulted in G1 arrest and cell size reduction, while NVP-BEZ235 resulted in aberrant cell cycle and multiple cell sizes (FIG. 11). WYE-354 also resulted in G1 arrest/cell size decrease in Leishmania. In contrast, while Pp242 showed G1 arrest/cell size decrease in trypanosomes, this was only found at lower drug concentrations in L. major, and at higher drug concentrations G2 arrest and increased cell size was observed instead. Thus, while the effects of specific inhibitors on trypanosomatids may resemble those seen against mammalian cells targeting specific TOR or PI3K targets, future studies will be required to more definitely establish the true mode(s) of action against the individual parasite species, which may differ.

It appears that the mTOR/PI3K inhibitors display generally superior activity against trypanosomatid growth over mTOR-selective inhibitors. This may be suggestive of the effect being mediated via inhibition of multiple trypanosomal PI3Ks, including PI3KKs such as TOR. With that in mind, efforts to identify the mechanism of action of these mTOR inhibitors in trypanosomatids will direct further medicinal chemistry efforts. Despite the lack of certainty of the mechanism of action of these compounds, the results provide a validation for the repurposing approach as a screening tool as an efficient approach to identification of compounds that are effective in parasite killing.

### REFERENCES

1. (2001) Human African trypanosomiasis: a guide for drug supply. World Health Organization
2. Chappuis F, Sundar S, Hailu A, Ghalib H, Rijal S, et al. (2007) Visceral leishmaniasis: what are the needs for diagnosis, treatment and control? Nat Rev Microbiol 5: 873-882.
3. Castro J A, de Mecca M M, Bartel L C (2006) Toxic side effects of drugs used to treat Chagas' disease (American trypanosomiasis). Hum Exp Toxicol 25: 471-479.
4. de Koning H P (2008) Ever-increasing complexities of diamidine and arsenical crossresistance in African trypanosomes. Trends Parasitol 24: 345-349.
5. Secor W E, Nguyen-Dinh P (2007) Mechanisms of resistance to antiparasitic agents. Man Clin Microbiol (9th Ed) 2: 2240-2249.
6. Wilkinson S R, Taylor M C, Horn D, Kelly J M, Cheeseman I (2008) A mechanism for cross-resistance to nifurtimox and benznidazole in trypanosomes. Proc Natl Acad Sci USA, Early Ed: 1-6, 6 pp.
7. Nwaka S, Hudson A (2006) Innovative lead discovery strategies for tropical diseases. Nat Rev Drug Discov 5: 941-955.
8. Frearson J A, Brand S, McElroy S P, Cleghorn L A T, Smid O, et al. (2010) N-myristoyltransferase inhibitors as new leads to treat sleeping sickness. Nature 464: 728-732.
9. Andrews K T, Walduck A, Kelso M J, Fairlie D P, Saul A, et al. (2000) Anti-malarial effect of histone deacetylation inhibitors and mammalian tumour cytodifferentiating agents. International Journal for Parasitology 30: 761-768.
10. Eastman R T, White J, Hucke O, Bauer K, Yokoyama K, et al. (2005) Resistance to a Protein Farnesyltransferase Inhibitor in Plasmodium falciparum. Journal of Biological Chemistry 280: 13554-13559.
11. Hopkins A L, Groom C R (2002) The druggable genome. Nat Rev Drug Discov 1: 727-730.
12. Ihle N T, Powis G (2009) Take your PIK: phosphatidylinositol 3-kinase inhibitors race through the clinic and toward cancer therapy. Mol Cancer Ther 8: 1-9.
13. Harris S J, Foster J G, Ward S G (2009) PI3K isoforms as drug targets in inflammatory diseases: lessons from pharmacological and genetic strategies. Curr Opin Invest Drugs (BioMed Cent) 10: 1151-1162.
14. Marone R, Cmiljanovic V, Giese B, Wymann M P (2008) Targeting phosphoinositide 3-kinase—Moving towards therapy. Biochimica et Biophysica Acta (BBA)—Proteins & Proteomics 1784: 159-185.
15. Choi J, Chen J, Schreiber S L, Clardy J (1996) Structure of the FKBP12-rapamycin complex interacting with the binding domain of human FRAP. Science 273: 239-242.
16. Liang J, Choi J, Clardy J (1999) Refined structure of the FKBP12-rapamycin-FRB ternary complex at 2.2 A resolution. Acta Crystallogr D Biol Crystallogr 55: 736-744.
17. Albert S, Serova M, Dreyer C, Sablin M-P, Faivre S, et al. (2010) New inhibitors of the mammalian target of rapamycin signaling pathway for cancer. Expert Opin Invest Drugs 19: 919-930.
18. Feldman M E, Apsel B, Uotila A, Loewith R, Knight Z A, et al. (2009) Active-site inhibitors of mTOR target rapamycin-resistant outputs of mTORC1 and mTORC2. PLoS Biol 7: e38.
19. Apsel B, Blair J A, Gonzalez B, Nazif T M, Feldman M E, et al. (2008) Targeted polypharmacology: discovery of dual inhibitors of tyrosine and phosphoinositide kinases. Nat Chem Biol 4: 691-699.
20. Zask A, Verheijen J C, Curran K, Kaplan J, Richard D J, et al. (2009) ATP-Competitive Inhibitors of the Mammalian Target of Rapamycin: Design and Synthesis of Highly Potent and Selective Pyrazolopyrimidines. J Med Chem 52: 5013-5016.
21. Garcia-Martinez J M, Moran J, Clarke R G, Gray A, Cosulich S C, et al. (2009) Ku-0063794 is a specific inhibitor of the mammalian target of rapamycin (mTOR). Biochem J 421: 29-42.
22. Malagu K, Duggan H, Menear K, Hummersone M, Gomez S, et al. (2009) The discovery and optimisation of pyrido[2,3-d]pyrimidine-2,4-diamines as potent and selective inhibitors of mTOR kinase. Bioorg Med Chem Lett 19: 5950-5953.
23. Nuss J M, Tsuhako A L, Anand N K (2009) Emerging therapies based on inhibitors of phosphatidyl-inositol-3-kinases. Annu Rep Med Chem 44: 339-356, 332 plates.
24. Guertin D A, Sabatini D M (2009) The pharmacology of mTOR inhibition. Sci Signal 2: pe24.
25. Verheijen J, Yu K, Zask A (2008) mTOR Inhibitors in Oncology. Annu Rep Med Chem 43: 189-202.
26. Bahia D, Oliveira L M, Lima F M, Oliveira P, Silveira J F, et al. (2009) The TryPIKinome of five human pathogenic trypanosomatids: Trypanosoma brucei, Trypanosoma cruzi, Leishmania major, Leishmania braziliensis and Leishmania infantum—new tools for designing specific inhibitors. Biochem Biophys Res Commun 390: 963-970.
27. Brown J R, Auger K R Phylogenomics of phosphoinositide lipid kinases: perspectives on the evolution of second messenger signaling and drug discovery. BMC Evol Biol 11: 4.
28. Hall B S, Gabernet-Castello C, Voak A, Goulding D, Natesan S K, et al. (2006) TbVps34, the trypanosome orthologue of Vps34, is required for Golgi complex segregation. J Biol Chem 281: 27600-27612.
29. Barquilla A, Crespo J L, Navarro M (2008) Rapamycin inhibits trypanosome cell growth by preventing TOR complex 2 formation. Proc Natl Acad Sci USA 105: 14579-14584.
30. Madeira da Silva L, Beverley S M (2010) Expansion of the target of rapamycin (TOR) kinase family and function in Leishmania shows that TOR3 is required for acidocalcisome biogenesis and animal infectivity. Proc Natl Acad Sci USA 107: 11965-11970.
31. de Jesus T C, Tonelli R R, Nardelli S C, da Silva Augusto L, Motta M C, et al. (2010) Target of rapamycin (TOR)-like 1 kinase is involved in the control of polyphosphate levels and acidocalcisome maintenance in Trypanosoma brucei. J Biol Chem 285: 24131-24140.
32. Polak P, Hall M N (2009) mTOR and the control of whole body metabolism. Curr Opin Cell Biol 21: 209-218.
33. Loewith R, Jacinto E, Wullschleger S, Lorberg A, Crespo J L, et al. (2002) Two TOR Complexes, Only One of which Is Rapamycin Sensitive, Have Distinct Roles in Cell Growth Control. Molecular cell 10: 457-468.
34. Soulard A, Cohen A, Hall M N (2009) TOR signaling in invertebrates. Curr Opin Cell Biol 21: 825-836.
35. Barquilla A, Navarro M (2009) Trypanosome TOR complex 2 functions in cytokinesis. Cell Cycle 8: 697-699.
36. Madeira da Silva L, Owens K L, Murta S M, Beverley S M (2009) Regulated expression of the Leishmania major surface virulence factor lipophosphoglycan using conditionally destabilized fusion proteins. Proc Natl Acad Sci USA 106: 7583-7588.
37. Turner C M, McLellan S, Lindergard L A, Bisoni L, Tait A, et al. (2004) Human infectivity trait in Trypanosoma brucei: stability, heritability and relationship to sra expression. Parasitology 129: 445-454.
38. Onyango J D, Burri C, Brun R (2000) An automated biological assay to determine levels of the trypanocidal drug melarsoprol in biological fluids. Acta Trop 74: 95-100.
39. Raz B, Iten M, Grether-Buhler Y, Kaminsky R, Brun R (1997) The Alamar Blue assay to determine drug sensitivity of African trypanosomes (T.b. rhodesiense and T.b. gambiense) in vitro. Acta Trop 68: 139-147.
40. Buckner F S, Verlinde C L, La Flamme A C, Van Voorhis W C (1996) Efficient technique for screening drugs for activity against Trypanosoma cruzi using parasites expressing beta-galactosidase. Antimicrob Agents Chemother 40: 2592-2597.
41. Kapler G M, Coburn C M, Beverley S M (1990) Stable transfection of the human parasite Leishmania major delineates a 30-kilobase region sufficient for extrachromosomal replication and expression. Mol Cell Biol 10: 1084-1094.
42. Goyard S, Segawa H, Gordon J, Showalter M, Duncan R, et al. (2003) An in vitro system for developmental and genetic studies of Leishmania donovani phosphoglycans. Mol Biochem Parasitol 130: 31-42.
43. Akopyants N S, Kimblin N, Secundino N, Patrick R, Peters N, et al. (2009) Demonstration of genetic exchange during cyclical development of Leishmania in the sand fly vector. Science 324: 265-268.
44. Cruz A K, Titus R, Beverley S M (1993) Plasticity in chromosome number and testing of essential genes in Leishmania by targeting. Proc Natl Acad Sci USA 90: 1599-1603.
45. Maira S M, Stauffer F, Brueggen J, Furet P, Schnell C, et al. (2008) Identification and characterization of NVP-BEZ235, a new orally available dual phosphatidylinositol 3-kinase/mammalian target of rapamycin inhibitor with potent in vivo antitumor activity. Mol Cancer Ther 7: 1851-1863.
46. Thalhofer C J, Graff J W, Love-Homan L, Hickerson S M, Craft N, et al. (2010) In vivo imaging of transgenic Leishmania parasites in a live host. J Vis Exp.
47. Spath G F, Beverley S M (2001) A lipophosphoglycan-independent method for isolation of infective Leishmania metacyclic promastigotes by density gradient centrifugation. Exp Parasitol 99: 97-103.
48. Yu K, Toral-Barza L, Shi C, Zhang W G, Lucas J, et al. (2009) Biochemical, cellular, and in vivo activity of novel ATP-competitive and selective inhibitors of the mammalian target of rapamycin. Cancer Res 69: 6232-6240.
49. Garlich J R, De P, Dey N, Su J D, Peng X, et al. (2008) A vascular targeted pan phosphoinositide 3-kinase inhibitor prodrug, SF1126, with antitumor and antiangiogenic activity. Cancer Res 68: 206-215.
50. Kristof A S, Pacheco-Rodriguez G, Schremmer B, Moss J (2005) LY303511 (2-piperazinyl-8-phenyl-4H-1-benzopyran-4-one) acts via phosphatidylinositol 3-kinase-independent pathways to inhibit cell proliferation via mammalian target of rapamycin (mTOR)- and non-mTOR-dependent mechanisms. J Pharmacol Exp Ther 314: 1134-1143.
51. Ding J, Vlahos C, Liu R, Brown R, Badwey J (1995) Antagonists of phosphatidylinositol 3-kinase block activation of several novel protein kinases in neutrophils. J Biol Chem 270: 11684-11691.
52. Fan Q W, Cheng C K, Nicolaides T P, Hackett C S, Knight Z A, et al. (2007) A dual phosphoinositide-3-kinase alpha/mTOR inhibitor cooperates with blockade of epidermal growth factor receptor in PTEN-mutant glioma. Cancer Res 67: 7960-7965.
53. Fan Q W, Knight Z A, Goldenberg D D, Yu W, Mostov K E, et al. (2006) A dual PI3 kinase/mTOR inhibitor reveals emergent efficacy in glioma. Cancer Cell 9: 341-349.
54. Knight Z A, Gonzalez B, Feldman M E, Zunder E R, Goldenberg D D, et al. (2006) A pharmacological map of the PI3-K family defines a role for p110alpha in insulin signaling. Cell 125: 733-747.
55. Ballou L M, Selinger E S, Choi J Y, Drueckhammer D G, Lin R Z (2007) Inhibition of mammalian target of rapamycin signaling by 2-(morpholin-1-yl)pyrimido[2,1-alpha]isoquinolin-4-one. J Biol Chem 282: 24463-24470.
56. McMillin D W, Ooi M, Delmore J, Negri J, Hayden P, et al. (2009) Antimyeloma activity of the orally bioavailable dual phosphatidylinositol 3-kinase/mammalian target of rapamycin inhibitor NVP-BEZ235. Cancer Res 69: 5835-5842.
57. Liu T J, Koul D, LaFortune T, Tiao N, Shen R J, et al. (2009) NVP-BEZ235, a novel dual phosphatidylinositol 3-kinase/mammalian target of rapamycin inhibitor, elicits multifaceted antitumor activities in human gliomas. Mol Cancer Ther 8: 2204-2210.
58. Tsang C K, Qi H, Liu L F, Zheng X F (2007) Targeting mammalian target of rapamycin (mTOR) for health and diseases. Drug Discov Today 12: 112-124.
59. Robinson D R, Sherwin T, Ploubidou A, Byard E H, Gull K (1995) Microtubule polarity and dynamics in the control of organelle positioning, segregation, and cytokinesis in the trypanosome cell cycle. J Cell Biol 128: 1163-1172.
60. Barquilla A, Navarro M (2009) Trypanosome TOR as a major regulator of cell growth and autophagy. Autophagy 5: 256-258.
61. Cheekatla S, Aggarwal A, Naik S (2011) mTOR signaling pathway regulates the IL-12/IL-10 axis in Leishmania donovani infection. Medical Microbiology and Immunology: 1-10.
62. Kim D-H, Sarbassov D D, Ali S M, King J E, Latek R R, et al. (2002) mTOR Interacts with Raptor to Form a Nutrient-Sensitive Complex that Signals to the Cell Growth Machinery. Cell 110: 163-175.
63. Hara K, Maruki Y, Long X, Yoshino K-i, Oshiro N, et al. (2002) Raptor, a Binding Partner of Target of Rapamycin (TOR), Mediates TOR Action. Cell 110: 177-189.
64. Sarbassov D D, Ali S M, Kim D H, Guertin D A, Latek R R, et al. (2004) Rictor, a novel binding partner of mTOR, defines a rapamycin-insensitive and raptor-independent pathway that regulates the cytoskeleton. Curr Biol 14: 1296-1302.
65. Jacinto E, Loewith R, Schmidt A, Lin S, Ruegg M A, et al. (2004) Mammalian TOR complex 2 controls the actin cytoskeleton and is rapamycin insensitive. Nat Cell Biol 6: 1122-1128.
66. Hayakawa M, Kaizawa H, Moritomo H, Koizumi T, Ohishi T, et al. (2007) Synthesis and biological evaluation of pyrido[3′,2′:4,5]furo[3,2-d]pyrimidine derivatives as novel PI3 kinase p110alpha inhibitors. Bioorg Med Chem Lett 17: 2438-2442.
67. Brunn G J, Williams J, Sabers C, Wiederrecht G, Lawrence J C, Jr., et al. (1996) Direct inhibition of the signaling functions of the mammalian target of rapamycin by the phosphoinositide 3-kinase inhibitors, wortmannin and LY294002. EMBO J 15: 5256-5267.
68. Vlahos C J, Matter W F, Hui K Y, Brown R F (1994) A specific inhibitor of phosphatidylinositol 3-kinase, 2-(4-morpholinyl)-8-phenyl-4H-1-benzopyran-4-one (LY294002). J Biol Chem 269: 5241-5248.
69. Griffin R J, Fontana G, Golding B T, Guiard S, Hardcastle I R, et al. (2005) Selective benzopyranone and pyrimido[2,1-a]isoquinolin-4-one inhibitors of DNA-dependent protein kinase: synthesis, structure-activity studies, and radiosensitization of a human tumor cell line in vitro. J Med Chem 48: 569-585.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A method of treating a disease caused by a trypanosomatid parasite, comprising administering a therapeutically effective amount of an mTOR and/or PI3K inhibitor compound to a subject in need of treatment, wherein the compound has the structure of Formula (I), 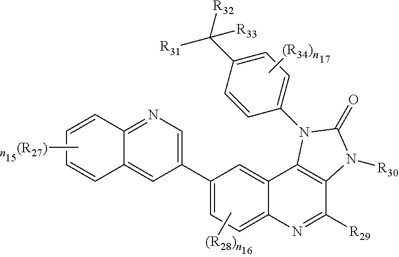
or a pharmaceutically acceptable salt or hydrate thereof,
wherein
R₂₇, R₂₈, R₂₉, and R₃₄ are each independently hydrogen, halogen, OH, CF₃, C₁-C₄ alkyl, ORₐ, OC(O)Rₐ, NRₐR_{b}, NRₐC(O)Rₐ, NRₐC(O)ORₐ, C(O)Rₐ, or C(O)NRₐR_{b};
R₃₀ is hydrogen, C₁-C₄ alkyl, aryl, heteroaryl, alkylaryl, alkylheteroaryl, C(O)Rₐ, C(O)ORₐR_{b}, or C(O)NRₐR_{b};
R₃₁ is hydrogen, halogen, OH, CN, CF₃, C₁-C₄ alkyl, ORₐ, OC(O)Rₐ, NRₐR_{b}, NRₐC(O)Rₐ, NRₐC(O)ORₐ, C(O)Rₐ, or C(O)NRₐR_{b};
R₃₂ and R₃₃ are each independently hydrogen or C₁-C₄ alkyl;
Rₐ and R_{b} are each independently hydrogen, C₁-C₄ alkyl, benzyl, pyridin-3-ylmethyl, —O—C₁-C₄ alkyl, or —C₁-C₄ alkyl-O—C₁-C₄ alkyl; and
n₁₅, n₁₆, and n₁₇ are each independently 0, 1, 2, 3, or 4.

2. The method of claim 1, wherein R₃₁ is CN.

3. The method of claim 1, wherein R₃₂ and R₃₃ are each H.

4. The method of claim 1, wherein R₃₀ is Me.

5. The method of claim 1, wherein R₂₇ is OMe, OH, or OAc.

6. The method of claim 1, wherein R₂₉ and R₃₄ are each H.

7. The method of claim 1, wherein the compound is

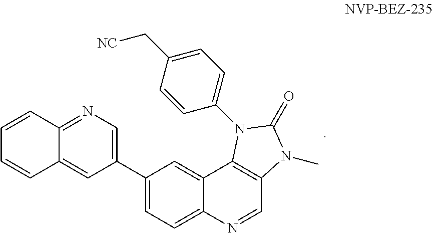 [SMILES: Cn1c(=O)n(-c2ccc(CC#N)cc2)c2c3cc(-c4cnc5ccccc5c4)ccc3ncc21]

8. A method of treating a disease caused by a trypanosomatid parasite, comprising administering a therapeutically effective amount of an mTOR and/or PI3K inhibitor compound to a subject in need of treatment, wherein the compound has the structure of Formula (VI), 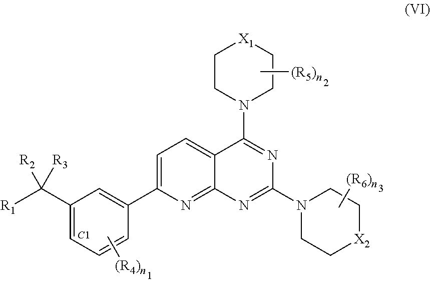
or a pharmaceutically acceptable salt or hydrate thereof,
wherein
R₁, R₂, R₃, and R₄ are each independently hydrogen, halogen, OH, CF₃, C₁-C₄ alkyl, ORₐ, NRₐR_{b}, C(O)Rₐ, or C(O)NRₐR_{b};
R₅ and R₆ are each independently hydrogen or C₁-C₄ alkyl;
X₁ and X₂ are each independently CRₐR_{b}, O, S, NRₐ, NC(O)Rₐ, or NC(O)ORₐ;
Ra and Rb are each independently hydrogen, C1-C4 alkyl, benzyl, pyridin-3-ylmethyl, —O—C₁-C₄ alkyl, or —C₁-C₄ alkyl O—C₁-C₄ alkyl; and
n₁, n₂, and n₃ are each independently 0, 1, 2, 3, or 4.

9. The method of claim 8, wherein X₁ and X₂ are each independently O.

10. The method of claim 8, wherein R₄ is attached to the C1 position of Formula (VI).

11. The method of claim 8, wherein R₄ is OMe.

12. The method of claim 8, wherein R₆ is Me.

13. The method of claim 8, wherein R₁ is OH.

14. The method of claim 8, wherein the compound is:

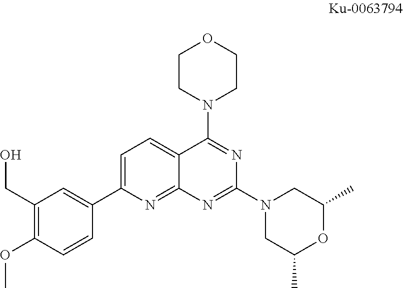 [SMILES: COc1ccc(-c2ccc3c(N4CCOCC4)nc(N4C[C@@H](C)O[C@@H](C)C4)nc3n2)cc1CO]

15. A method of treating a disease caused by a trypanosomatid parasite, comprising administering a therapeutically effective amount of an mTOR and/or PI3K inhibitor compound to a subject in need of treatment, wherein the compound has the structure of Formula (II), 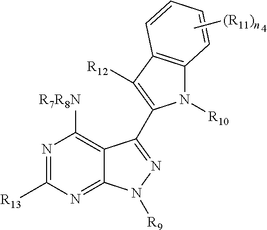
or a pharmaceutically acceptable salt or hydrate thereof,
wherein
R₇, R₈, R₉, and R₁₀ are each independently hydrogen, C₁-C₄ alkyl, aryl, heteroaryl, alkylaryl, alkylheteroaryl, C(O)Rₐ, C(O)ORₐ, or C(O)NRₐR_{b};
R₁₁, R₁₂ and R₁₃ are each independently hydrogen, halogen, OH, CF₃, C₁-C₄ alkyl, ORₐ, NRₐR_{b}, C(O)Rₐ, or C(O)NRₐR_{b};
Rₐ and R_{b} are each independently hydrogen, C₁-C₄ alkyl, benzyl, pyridin-3-ylmethyl, —O—C₁-C₄ alkyl; and
n₄ is 0, 1, 2, 3, or 4.

16. The method of claim 15, wherein R₇ and R₈ are H.

17. The method of claim 15, wherein R₁₃ is H.

18. The method of claim 15, wherein R₉ is methyl, ethyl, or isopropyl.

19. The method of claim 15, wherein R₁₀ is H.

20. The method of claim 15, wherein R₁₁ is OH.

21. The method of claim 15, wherein the compound is:

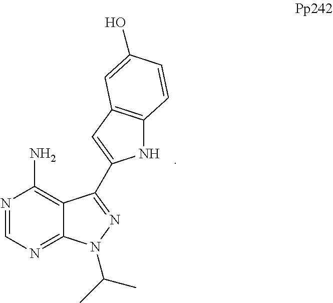 [SMILES: CC(C)n1nc(-c2cc3cc(O)ccc3[nH]2)c2c(N)ncnc21]

22. A method of treating a disease caused by a trypanosomatid parasite, comprising administering a therapeutically effective amount of an mTOR and/or PI3K inhibitor compound to a subject in need of treatment, wherein the compound has the structure of Formula (III), 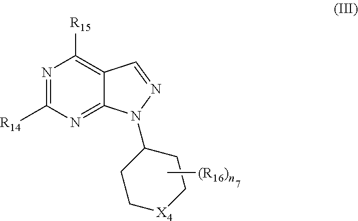
or a pharmaceutically acceptable salt or hydrate thereof,
wherein R₁₄ is hydrogen, —C₁-C₄ alkyl-NRₐC(O)ORₐ, or 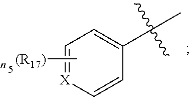
R₁₅ is hydrogen, C₁-C₄ alkyl, aryl, heteroaryl, NRₐR_{b}, or 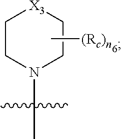
R_{c} and R₁₆ are each independently hydrogen or C₁-C₄ alkyl;
R₁₇ is hydrogen, halogen, OH, CF₃, C₁-C₄ alkyl, ORₐ, OC(O)Rₐ, NRₐR_{b}, NRₐC(O)Rₐ, NRₐC(O)ORₐ, C(O)Rₐ, or C(O)NRₐR_{b};
Rₐ and R_{b} are each independently hydrogen, C₁-C₄ alkyl, benzyl, pyridin-3-ylmethyl, —O—C₁-C₄ alkyl, or —C₁-C₄ alkyl O—C₁-C₄ alkyl;
X is C or N;
X₃ and X₄ are each independently CRₐR_{b}, O, S, NRₐ, NC(O)Rₐ, or NC(O)ORₐ; and
n₅, n₆, and n₇ are each independently 0, 1, 2, 3, or 4.

23. The method of claim 22, wherein X₄ is N-benzyl.

24. The method of claim 22, wherein X₄ is N-(pyridin-3-ylmethyl).

25. The method of claim 22, wherein X₄ is NC(O)OMe.

26. The method of claim 22, wherein X₄ is NH.

27. The method of claim 22, wherein R₁₄ is

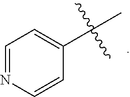 [SMILES: *c1ccncc1]

28. The method of claim 22, wherein R₁₇ is NH₂, OH, NHC(O)OMe, or NHC(O)OC(CH₃)₃.

29. The method of claim 22, wherein R₁₅ is H.

30. The method of claim 22, wherein R₁₅ is NHCH₂CH₂OCH₂CH₃.

31. The method of claim 22, wherein R₁₄ is CH₂CH₂NHC(O)OCH₃.

32. The method of claim 22, wherein X₃ is CH₂.

33. The method of claim 22, wherein X₃ is O.

34. The method of claim 22, wherein the compound is selected from the group consisting of:

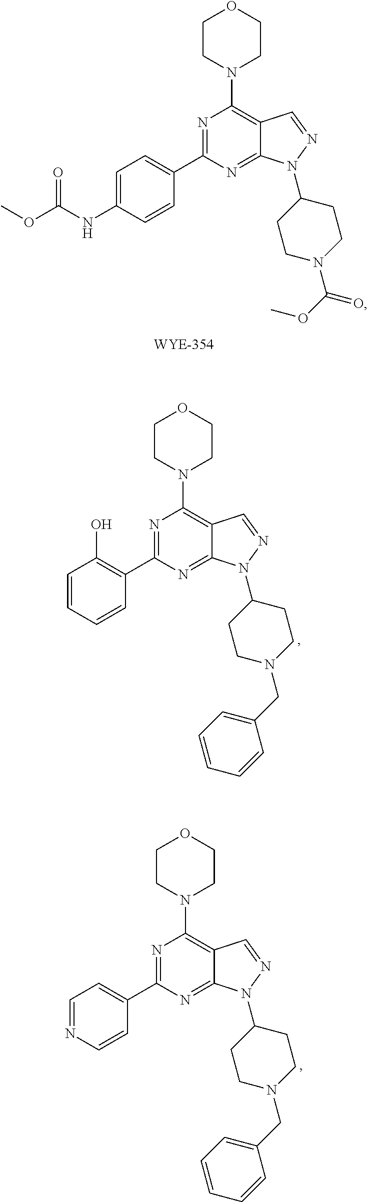 [SMILES: COC(=O)Nc1ccc(-c2nc(N3CCOCC3)c3cnn(C4CCN(C(=O)OC)CC4)c3n2)cc1 ; Oc1ccccc1-c1nc(N2CCOCC2)c2cnn(C3CCN(Cc4ccccc4)CC3)c2n1 ; c1ccc(CN2CCC(n3ncc4c(N5CCOCC5)nc(-c5ccncc5)nc43)CC2)cc1]

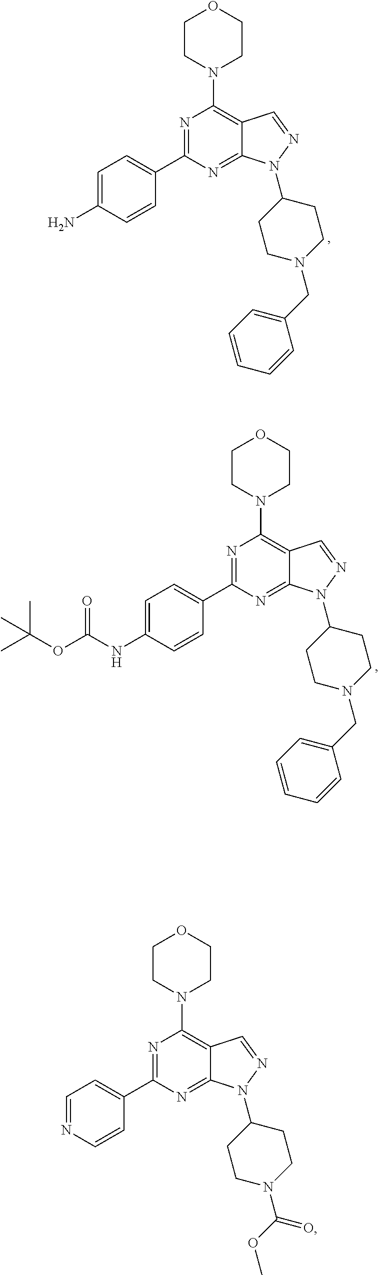 [SMILES: Nc1ccc(-c2nc(N3CCOCC3)c3cnn(C4CCN(Cc5ccccc5)CC4)c3n2)cc1 ; CC(C)(C)OC(=O)Nc1ccc(-c2nc(N3CCOCC3)c3cnn(C4CCN(Cc5ccccc5)CC4)c3n2)cc1 ; COC(=O)N1CCC(n2ncc3c(N4CCOCC4)nc(-c4ccncc4)nc32)CC1]

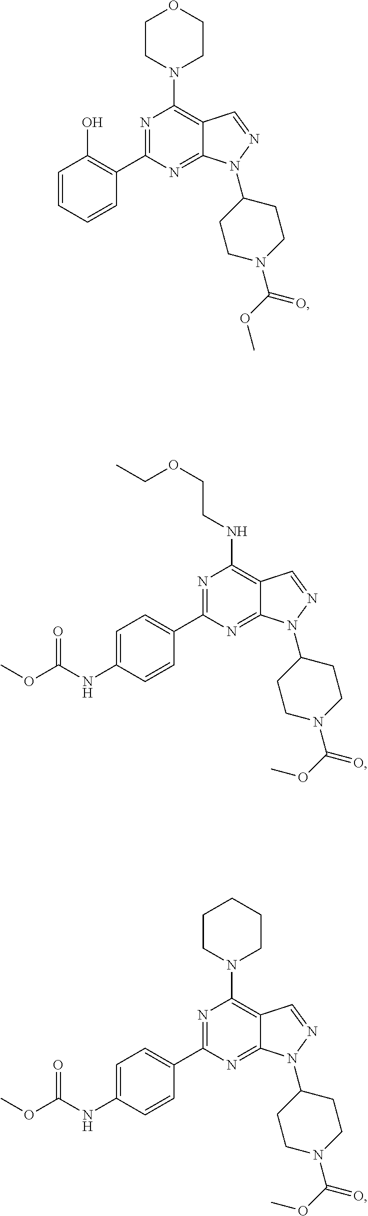 [SMILES: COC(=O)N1CCC(n2ncc3c(N4CCOCC4)nc(-c4ccccc4O)nc32)CC1 ; CCOCCNc1nc(-c2ccc(NC(=O)OC)cc2)nc2c1cnn2C1CCN(C(=O)OC)CC1 ; COC(=O)Nc1ccc(-c2nc(N3CCCCC3)c3cnn(C4CCN(C(=O)OC)CC4)c3n2)cc1]

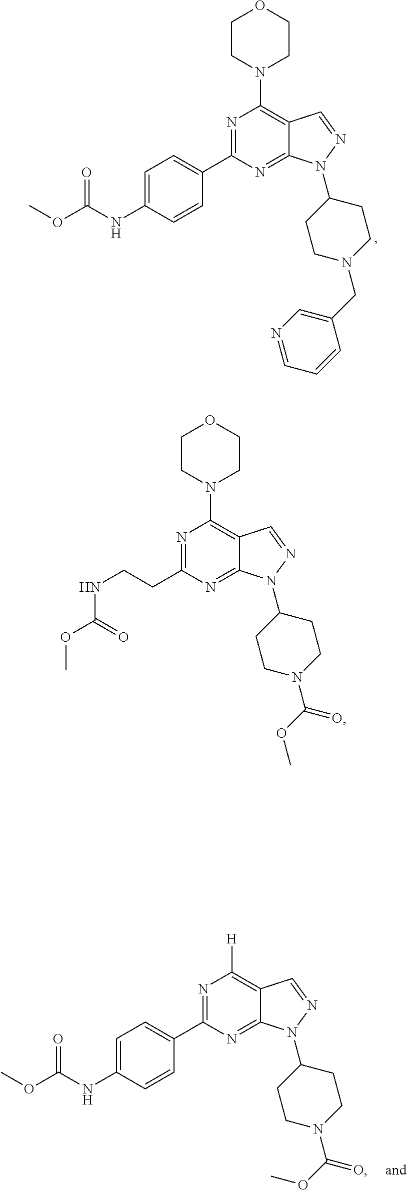

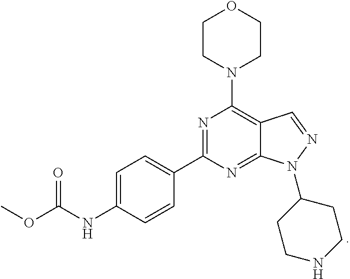 [SMILES: COC(=O)Nc1ccc(-c2nc(N3CCOCC3)c3cnn(C4CCNCC4)c3n2)cc1]

35. A method of treating a disease caused by a trypanosomatid parasite, comprising administering a therapeutically effective amount of an mTOR and/or PI3K inhibitor compound to a subject in need of treatment, wherein the compound has the structure of Formula (IV), 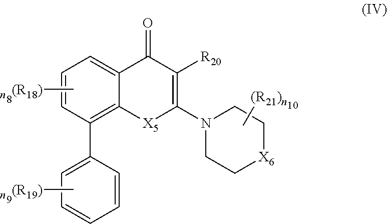
or a pharmaceutically acceptable salt or hydrate thereof,
wherein
R₁₈, R₁₉, and R₂₀ are each independently hydrogen, halogen, OH, CF₃, C₁-C₄ alkyl, ORₐ, OC(O)Rₐ, NRₐR_{b}, NRₐC(O)Rₐ, NRₐC(O)ORₐ, C(O)Rₐ, or C(O)NRₐR_{b};
R₂₁ is hydrogen or C₁-C₄ alkyl;
X₅ and X₆ are each independently CRₐR_{b}, O, S, NRₐ, NC(O)Rₐ, or NC(O)ORₐ;
Rₐ and R_{b} are each independently hydrogen, C₁-C₄ alkyl, benzyl, pyridin-3-ylmethyl, —O—C₁-C₄ alkyl, or —C₁-C₄ alkyl O—C₁-C₄ alkyl; and
n₈, n₉, and n₁₀ are each independently 0, 1, 2, 3, or 4.

36. The method of claim 35, wherein X₅ is O.

37. The method of claim 35, wherein X₆ is O.

38. The method of claim 35, wherein X₆ is NH.

39. The method of claim 35, wherein R₂₀ is H.

40. The method of claim 35, wherein the compound is selected from the group consisting of:

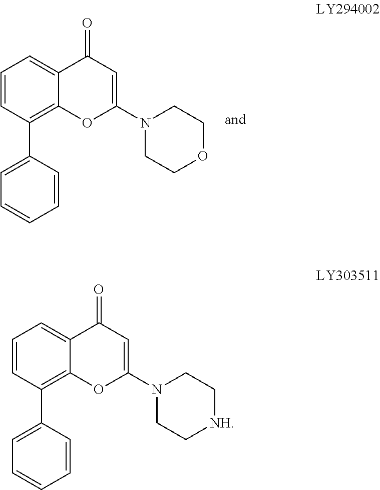 [SMILES: O=c1cc(N2CCOCC2)oc2c(-c3ccccc3)cccc12 ; O=c1cc(N2CCNCC2)oc2c(-c3ccccc3)cccc12]

41. A method of treating a disease caused by a trypanosomatid parasite, comprising administering a therapeutically effective amount of an mTOR and/or PI3K inhibitor compound to a subject in need of treatment, wherein the compound has the structure of Formula (V), 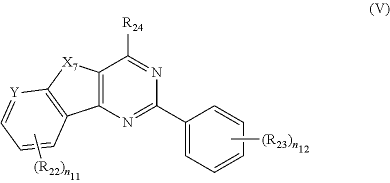
or a pharmaceutically acceptable salt or hydrate thereof,
wherein
R₂₄ is hydrogen, C₁-C₄ alkyl, aryl, heteroaryl, NRₐR_{b}, 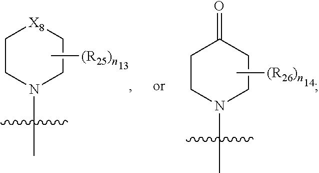
R₂₂ and R₂₃ are each independently hydrogen, halogen, OH, CF₃, C₁-C₄ alkyl, ORₐ, C₁-C₄ alkyl-ORₐ, OC(O)Rₐ, NRₐR_{b}, NRₐC(O)Rₐ, NRₐC(O)ORₐ, C(O)Rₐ, or C(O)NRₐR_{b};
R₂₅ and R₂₆ are each independently hydrogen or C₁-C₄ alkyl;
X₇ and X₈ are each independently CRₐR_{b}, O, S, NRₐ, NC(O)Rₐ, or NC(O)ORₐ;
Rₐ and R_{b} are each independently hydrogen, C₁-C₄ alkyl, benzyl, pyridin-3-ylmethyl, —O—C₁-C₄ alkyl, or —C₁-C₄ alkyl-O—C₁₋₄ alkyl;
Y is C or N; and
n₁₁, n₁₂, n₁₃, and n₁₄ are each independently 0, 1, 2, 3, or 4.

42. The method of claim 41, wherein R₂₄ is H.

43. The method of claim 41, wherein R₂₄ is NHCH₂CH₂OCH₂CH₃.

44. The method of claim 41, wherein R₂₄ is

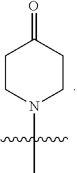 [SMILES: *N1CCC(=O)CC1]

45. The method of claim 41, wherein R₂₄ is

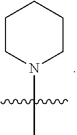 [SMILES: *N1CCCCC1]

46. The method of claim 41, wherein R₂₄ is

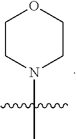 [SMILES: *N1CCOCC1]

47. The method of claim 41, wherein R₂₃ is H.

48. The method of claim 41, wherein R₂₃ is ortho-OEt.

49. The method of claim 41, wherein R₂₃ is meta-OMe, meta-OH, or meta-OAc.

50. The method of claim 41, wherein R₂₃ is para-OMe.

51. The method of claim 41, wherein R₂₃ is meta-CH₂CH₂OH.

52. The method of claim 41, wherein X₇ is S or O.

53. The method of claim 41, wherein Y is N.

54. The method of claim 41, wherein Y is C.

55. The method of claim 41, wherein the compound is selected from the consisting of:

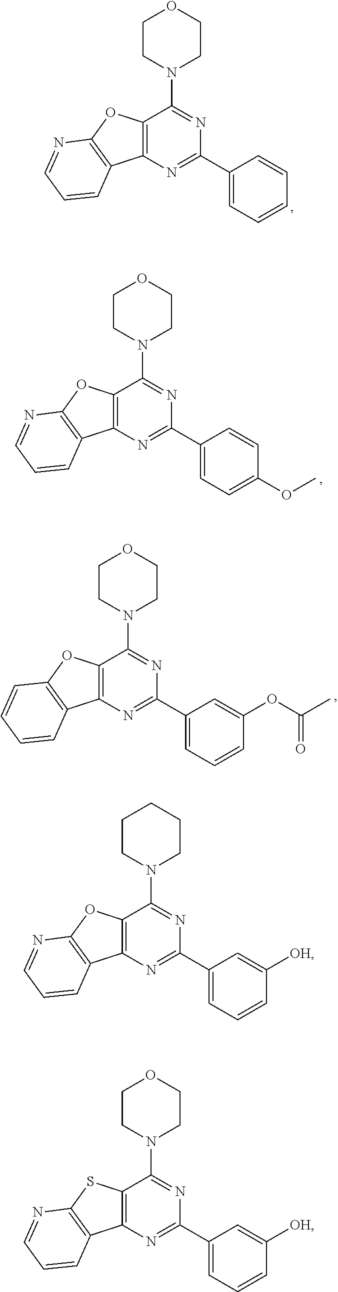 [SMILES: c1ccc(-c2nc(N3CCOCC3)c3oc4ncccc4c3n2)cc1 ; COc1ccc(-c2nc(N3CCOCC3)c3oc4ncccc4c3n2)cc1 ; CC(=O)Oc1cccc(-c2nc(N3CCOCC3)c3oc4ccccc4c3n2)c1 ; Oc1cccc(-c2nc(N3CCCCC3)c3oc4ncccc4c3n2)c1 ; Oc1cccc(-c2nc(N3CCOCC3)c3sc4ncccc4c3n2)c1]

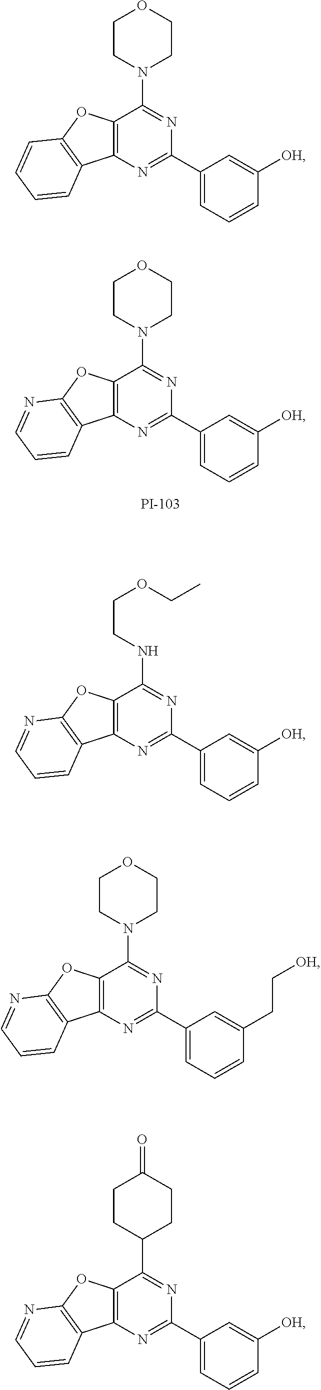 [SMILES: Oc1cccc(-c2nc(N3CCOCC3)c3oc4ccccc4c3n2)c1 ; Oc1cccc(-c2nc(N3CCOCC3)c3oc4ncccc4c3n2)c1 ; CCOCCNc1nc(-c2cccc(O)c2)nc2c1oc1ncccc12 ; OCCc1cccc(-c2nc(N3CCOCC3)c3oc4ncccc4c3n2)c1 ; O=C1CCC(c2nc(-c3cccc(O)c3)nc3c2oc2ncccc23)CC1]

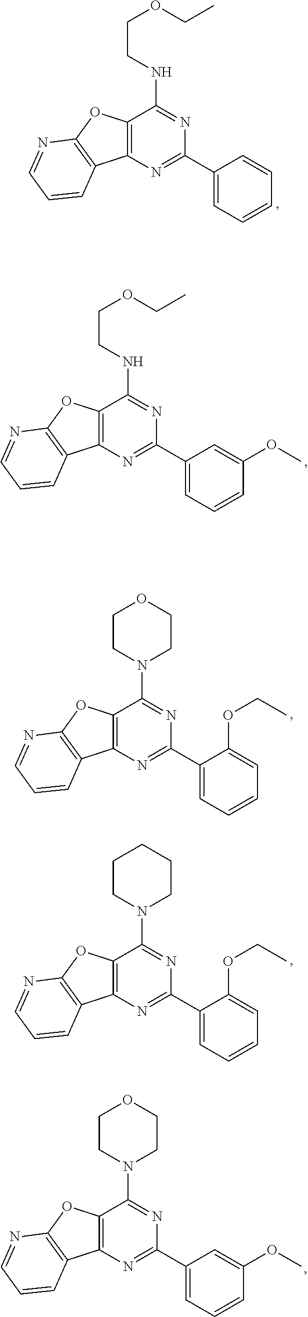 [SMILES: CCOCCNc1nc(-c2ccccc2)nc2c1oc1ncccc12 ; CCOCCNc1nc(-c2cccc(OC)c2)nc2c1oc1ncccc12 ; CCOc1ccccc1-c1nc(N2CCOCC2)c2oc3ncccc3c2n1 ; CCOc1ccccc1-c1nc(N2CCCCC2)c2oc3ncccc3c2n1 ; COc1cccc(-c2nc(N3CCOCC3)c3oc4ncccc4c3n2)c1]

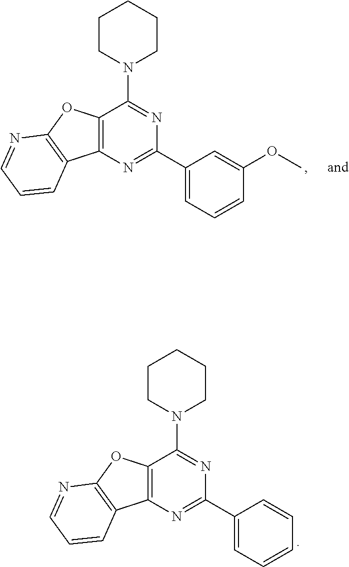 [SMILES: COc1cccc(-c2nc(N3CCCCC3)c3oc4ncccc4c3n2)c1 ; c1ccc(-c2nc(N3CCCCC3)c3oc4ncccc4c3n2)cc1]

56. A method of treating a disease caused by a trypanosomatid parasite, comprising administering a therapeutically effective amount of an mTOR and/or PI3K inhibitor compound to a subject in need of treatment, wherein the compound has the structure of Formula (VII), 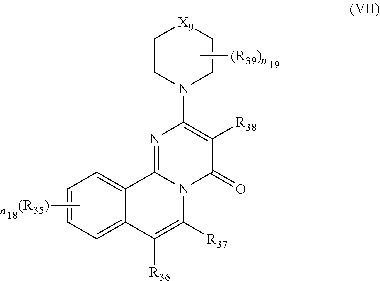
or a pharmaceutically acceptable salt or hydrate thereof,
wherein
R₃₅, R₃₆, R₃₇, and R₃₈ are each independently hydrogen, halogen, OH, CF₃, C₁-C₄ alkyl, ORₐ, OC(O)Rₐ, NRₐR_{b}, NRₐC(O)Rₐ, NRₐC(O)ORₐ, C(O)Rₐ, or C(O)NRₐR_{b};
R₃₉ is hydrogen or C₁-C₄ alkyl;
X₉ is CRₐR_{b}, O, S, NRₐ, NC(O)Rₐ, or NC(O)ORₐ;
Rₐ and R_{b} are each independently hydrogen, C₁-C₄ alkyl, benzyl, pyridin-3-ylmethyl, —O—C₁-C₄ alkyl, or —C₁-C₄ alkyl O—C₁-C₄ alkyl; and
n₁₈ and n₁₉ are each independently 0, 1, 2, 3, or 4.

57. The method of claim 56, wherein R₃₅ is H or OMe.

58. The method of claim 56, wherein X₉ is O.

59. The method of claim 56, wherein the compound is

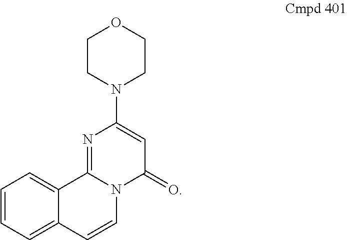 [SMILES: O=c1cc(N2CCOCC2)nc2c3ccccc3ccn12]

60. The method of any one of claims 1, 8, 15, 22, 35, 41, and 56, wherein the disease is selected from the group consisting of Human African Trypanosomiasis, leishmaniasis, and Chagas Disease.

61. The method of claim 60, wherein the Human African Trypanosomiasis is caused by Trypanosoma brucei.

62. The method of claim 60, wherein the leishmaniasis is caused by Leishmania sp.

63. The method of claim 60, wherein leishmaniasis is visceral or cutaneous leishmaniasis.

64. The method of claim 60, wherein the Chagas Disease is caused by Trypanosoma cruzi.

65-66. (canceled)
